(19) United States
(12) Patent Application Publication
(10) Pub. No.: US 2022/0409697 A1
(43) Pub. Date: Dec. 29, 2022

(54) Extended Half-life G-CSF and GM-CSF Vitamin D Conjugates

(71) Applicants: RAMEA LLC, Boston, MA (US); Extend Biosciences Inc., Newton, MA (US)

(72) Inventors: Russell J. Barron, Boston, MA (US); Tarik Soliman, Cambridge, MA (US); Daniel B. Hall, Cambridge, MA (US)

(73) Assignees: RAMEA LLC, Boston, MA (US); Extend Biosciences Inc., Newton, MA (US)

(21) Appl. No.: 17/769,916

(22) PCT Filed: Oct. 19, 2020
(86) PCT No.: PCT/US20/56210

§ 371 Date: Apr. 18, 2022

Related U.S. Application Data
(60) Provisional application No. 62/923,498, filed on Oct. 19, 2019.

(51) Int. Cl. A61K 38/19; A61K 47/55

(52) U.S. Cl. CPC A61K 38/193; A61K 47/551

(57) ABSTRACT

The invention provides non-hormonal Vitamin D conjugated to G-CSF or compounds with G-CSF activity or GM-CSF or compounds having GM-CSF activity singly or in combination that result in increased absorption, bioavailability or circulating half-life when compared to non-conjugated forms.

### RELATED APPLICATION

This application claims the benefit of U.S. Provisional Application No. 62/923,498, entitled, “Extended Half-Life G-CSF Vitamin D Conjugates” by Russell J Barron, filed Oct. 19, 2019; and this application is a Continuation in part of Ser. No. 16/920,652 filed on Jul. 3, 2020, entitled “Therapeutic Vitamin D Conjugates” by Tarik Soliman et al., and a Continuation in Part of Ser. No. 16/261,507 filed on Jan. 29, 2019, which is a continuation of Ser. No. 16/034,046 filed on Jul. 12, 2018, now U.S. Pat. No. 10,406,202, issued on Sep. 10, 2019, entitled “Therapeutic Vitamin D Conjugates” by Tarik Soliman et al., which is a continuation of Ser. No. 15/430,449 filed on Feb. 11, 2017, now U.S. Pat. No. 10,702,574, issued on Jul. 7, 2020, entitled “Therapeutic Vitamin D Conjugates” by Tarik Soliman et al., which is a continuation of Ser. No. 14/919,601 filed on Oct. 21, 2015, now U.S. Pat. No. 9,585,934, issued Mar. 7, 2017, entitled “Therapeutic Vitamin D Conjugates” by Tarik Soliman et al., which claims the benefit of U.S. Application No. 62/244,181, filed Oct. 20, 2015, entitled “Therapeutic Vitamin D Conjugates” by Tarik Soliman et al. and claims the benefit of U.S. Application No. 62/067,388, filed on Oct. 22, 2014, entitled “Therapeutic Vitamin D Conjugates” by Tarik Soliman et al.

The entire teachings of the above applications are incorporated herein by reference.

### FIELD OF THE INVENTION

The invention provides non-hormonal vitamin D conjugates of G-CSF and GM-CSF proteins individually or in combination that result in increased absorption, bioavailability or circulating half-life when compared to non-circulating forms. In some embodiments, the vitamin D targeting groups are coupled to the proteins via the third carbon of the vitamin D backbone.

### BACKGROUND OF THE INVENTION

Absorption is a primary focus in drug development and medicinal chemistry since a drug must be absorbed before any medicinal effects can take place. A drug's pharmacokinetic profile can be affected by many factors. Additionally, the absorption properties of therapeutic compounds vary significantly from compound to compound. Some therapeutic compounds are poorly absorbed following oral or dermal administration. Other therapeutic compounds, such as most peptide- and protein-based therapeutics, cannot be administered orally. Alternate routes of administration such as intravenous, subcutaneous, or intramuscular injections are routinely used for some of compounds; however, these routes often result in slow absorption and exposure of the therapeutic compounds to enzymes that can degrade them, thus requiring much higher doses to achieve efficacy.

A number of compounds such as G-CSF and GM-CSF have been identified as therapeutically important. However, each suffers from extremely short half-life. G-CSF is variously reported to have a half-life of ca. 2 hours. The half-life of GM-CSF is even shorter and is variously reported to be a few minutes.

The chemical and biological properties of G-CSF make it important for use as a therapeutic compound. G-CSF is a naturally occurring molecule and are involved in numerous physiological processes including neutrapenia for which it is the standard of care for post-cancer chemotherapy. G-CSF displays a high degree of selectivity and potency and may suffer from potential adverse drug-drug interactions or other negative side effects. C-CSF has a short in vivo half-life of approximately 3.5 hours or less. This may render it undesirably impractical, in its native form or when pegalated, for therapeutic administration. Additionally, G-CSF has a short duration of action or poor bioavailability.

The chemical and biological properties of GM-CSF make it important for use as a therapeutic compound. G-CSF is a naturally occurring molecule and are involved in numerous physiological processes including neutrapenia for which it is a component of care. GM-CSF displays a high degree of selectivity and potency and may suffer from potential adverse drug-drug interactions or other negative side effects. CM-CSF has a very short in vivo half-life of approximately 10 minutes or less. This may render it undesirably impractical, in its native form or when pegalated, for therapeutic administration. Additionally, GM-CSF has a short duration of action or poor bioavailability.

A need exists to modify G-CSF and GM-CSF to increase their half-lifes. A further need exists to improve their use as therapeutic compounds when used singly or in combination and specifically improve, absorption, stability, half-life, duration of effect, potency, or bioavailability.

### SUMMARY OF THE INVENTION

The invention provides carriers conjugated to molecules including G-CSF or GM-CSF and biosimilars and interchangeables (e.g., variants, homologues and/or analogs) of those or compounds having G-CSF or GM-CSF activity. The conjugated molecule enhances the respective activity of G-CSF or GM-CSF singly or when used in combination including but not limited to the absorption, stability, half-life, duration of effect, potency, or bioavailability. The carriers comprise targeting groups that bind the Vitamin D Binding protein (DBP), conjugation groups for coupling the targeting groups to the therapeutic compounds, and optional scaffolding moieties. See FIG. 1.

The invention improves the potency, absorption or pharmacokinetic properties of therapeutic compounds to certain vitamin D forms. Vitamin D plays a role in calcium, phosphate, and bone homeostasis. The hormonal activity of vitamin D is mediated through binding to the vitamin D receptor (VDR). It enters the nucleus where it binds to the vitamin D receptor element (VDRE) present in the promoters of a subset of genes that are thus responsive to hormonal Vitamin D. Vitamin D is a group of fat-soluble secosteroids. Several forms (vitamers) of vitamin D exist. The two major forms are vitamin D2 or ergocalciferol, and vitamin D3 or cholecalciferol. Vitamin D without a subscript refers to vitamin D2, D3 or other forms known in the art. In humans, vitamin D can be ingested as cholecalciferol (vitamin D3) or ergocalciferol (vitamin D2). The major source of vitamin D for most humans is sunlight. Once vitamin D is made in the skin or ingested, it needs to be activated by a series of hydroxylation steps, first to 25-hydroxyvitamin D (25(OH)D3) in the liver and then to 1,25-dihydroxyvitamin D3 (1α,25(OH)2D3) in the kidney. 1α,25(OH)2D3 is the active “hormonal” form of vitamin D because it binds to VDR. 25(OH)D3 is the “non-hormonal” form of vitamin D and is the major circulating form in the human body. It binds the vitamin D Binding Protein (DBP). It is only converted to the hormonal form as needed. An example of a non-hormonal vitamin D form is one that lacks a la-hydroxyl group. Non-hormonal vitamin D forms have a greatly reduced affinity for VDR and a greatly increased affinity for DBP.

DBP is the principal transporter of vitamin D metabolites. Its concentration in the plasma is 6-7 μM and has been detected in all fluid compartments. DBP concentrations exceed the physiological vitamin D metabolite concentrations. DBP is important for the translocation of vitamin D from the skin into circulation, and across cell membranes into the cytoplasm where vitamin D is activated into the hormonal form. The affinity of non-hormonal Vitamin D for DBP is significantly higher than the affinity of the hormonal form. In contrast, the affinity of the hormonal form to VDR is significantly than the non-hormonal form.

Vitamin D and vitamin D analogs have been approved for the treatment of osteoporosis and secondary hyperparathyroidism. Vitamin D has also been shown to inhibit proliferation and induce differentiation in normal as well as cancer cells. The level of vitamin D required for this activity causes severe toxicity in the form of hypercalcemia. Analogs of vitamin D have been approved for the treatment of psoriasis and others are currently being tested for cancer treatment. Many of the analogs discovered to have a reduced calcemic effect contain side-chain modifications. These modifications do not greatly affect VDR binding, and thus, in cell-based proliferation assays, show equal or even increased efficacy. It was shown, however, that many of these modifications reduce binding to DBP and thereby reduce the half-life in the bloodstream. Absorption is a primary focus in drug development and medicinal chemistry because a drug must be absorbed before any medicinal effects can take place. A drug's absorption profile can be affected by many factors. Additionally, the absorption properties of therapeutic compounds vary significantly from compound to compound. Some therapeutic compounds are poorly absorbed following oral or dermal administration. Other therapeutic compounds, such as most peptide- and protein-based therapeutics, cannot be administered orally. Alternate routes of administration such as intravenous, subcutaneous, or intramuscular injections are routinely used for some of these compounds; however, these routes often result in slow absorption and exposure of the therapeutic compounds to enzymes that can degrade them, thus requiring much higher doses to achieve efficacy.

A number of compounds have been identified as therapeutically promising. The chemical and biological properties of peptides, proteins and factors such as G-CSF and GM-CSF make them attractive candidates for use as therapeutic compounds singly or in combination. Peptides, proteins including G-CSF and GM-CSF are naturally occurring molecules and are involved in numerous physiological processes. They display a high degree of selectivity and potency and may not suffer from potential adverse drug-drug interactions or other negative side effects. Thus they hold great promise as a highly diverse, highly potent, and highly selective class of therapeutic compounds with low toxicity. Unfortunately, however, they may have short in vivo half-lives. For such molecules, this may be a few minutes. This may render them generally impractical, in their native form (also referred to as “wild”, “wild type” or “wt” herein), for therapeutic administration. Additionally, they may have a short duration of action or poor bioavailability.

In an embodiment of the invention, the targeting group is vitamin D, a vitamin D analog, a vitamin D-related metabolite, an analog of a vitamin D related-metabolite, a peptide that binds DBP, an anti-DBP antibody, an anti-DBP antibody derivative, a nucleotide aptamer that binds DBP, or a small carbon-based molecule that binds DBP.

In another embodiment, the coupling group is an amine-reactive group, a thiol-reactive group, a maleimide group, a thiol group, an aldehyde group, an NHS-ester group, a 4-nitrophenyl ester, an acylimidazole, a haloacetyl group, an iodoacetyl group, a bromoacetyl groups, a SMCC group, a sulfo SMCC group, a carbodiimide group and bifunctional cross-linkers such as NHS-Maleimido or combinations thereof. The coupling groups of the invention can promote thiol linkages, amide linkages, oxime linkages, hydrazone linkages, thiazolidinone linkages or utilizes cycloaddition reactions (e.g. click chemistry) to couple the carrier or targeting group to a therapeutic compound.

In another embodiment, the pharmaceutical carrier further comprising a scaffold moiety, comprising poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, or an additional therapeutic moiety.

In another embodiment, the scaffold moiety is between about 100 Da. and 200,000 Da. In preferred embodiments, the scaffold moiety is between about 100 Da. and 20,000 Da., 200 Da. and 15,000 Da., 300 Da. and 10,000 Da., 400 Da. and 9,000 Da., 500 Da. and 5,000 Da., 600 Da. and 2,000 Da., 1000 Da. and 200,000 Da., 5000 Da. and 100,000 Da., 10,000 Da. and 80,000 Da., 20,000 Da. and 60,000 Da., or 20,000 Da. and 40,000 Da.

The invention provides a pharmaceutical composition comprising a therapeutic compound conjugated to, fused to, or formulated with a carrier. The carrier comprises a targeting group that binds DBP and increases the absorption, bioavailability, or half-life of the therapeutic compound in circulation. The pharmaceutical compositions of the invention may comprise two or more therapeutic compounds conjugated to a single carrier. The pharmaceutical compositions of the invention may comprise two or more carriers conjugated to a therapeutic compound.

In one embodiment, the targeting group in the pharmaceutical composition is vitamin D, a vitamin D analog, a vitamin D-related metabolite, an analog of a vitamin D-related metabolite, a peptide that binds DBP, an anti-DBP antibody, an anti-DBP antibody derivative, a nucleotide aptamer that binds DBP, or a small, carbon-based molecule that binds DBP.

In another embodiment, the pharmaceutical composition further comprises a scaffold moiety. In a preferred embodiment, the scaffold moiety is poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, or an additional therapeutic compound.

The pharmaceutical compositions of the invention may comprise small molecules, chemical entities, nucleic acids, nucleic acid derivatives, peptides, peptide derivatives, naturally-occurring proteins, non-naturally-occurring proteins, peptide-nucleic acids (PNA), stapled peptides, morpholinos, phosphorodiamidate morpholinos, antisense drugs, RNA-based silencing drugs, aptamers, glycoproteins, enzymes, hormones, cytokines, interferons, growth factors, blood coagulation factors, antibodies, antibody fragments, antibody derivatives, toxin-conjugated antibodies, metabolic effectors, analgesics, antipyretics, anti-inflammatory agents, antibiotics, anti-microbial agents, anti-viral agents, anti-fungal drugs, musculoskeletal drugs, cardiovascular drugs, renal drugs, pulmonary drugs, digestive disease drugs, hematologic drugs, urologic drugs, metabolism drugs, hepatic drugs, neurological drugs, anti-diabetes drugs, anti-cancer drugs, drugs for treating stomach conditions, drugs for treating colon conditions, drugs for treating skin conditions, drugs for treating lymphatic conditions or G-CSF of compounds having G-CSF activity or GM-CSF or compounds having GM-CSF activity.

In a preferred embodiment, the pharmaceutical composition comprises a protein having G-CSF activity comprising an amino acid sequence with at least a about 90% identity to SEQ ID NO: 2, 4, 6, 8, or 10, or about 90% similarity to SEQ ID NO: 2, 4, 6, 8, or 10. In another preferred embodiment, the targeting group is Vitamin D. In another preferred embodiment, the scaffold moiety is poly(ethylene glycol).

In a most preferred embodiment, the invention contemplates a pharmaceutical composition comprising a protein having G-CSF activity comprising an amino acid sequence with at least about 90% identity to SEQ ID NO: 2, 4, 6, 8, or 10, or at least about 90% similarity with SEQ ID NO: 2, 4, 6, 8, or 10, a scaffold moiety that is poly(ethylene glycol), and a targeting group that is Vitamin D. In this embodiment, the targeting group increases the absorption, bioavailability, or the half-life of the therapeutic compound in circulation. In another most preferred embodiment, the invention contemplates a pharmaceutical composition comprising a protein having G-CSF activity and the amino acid sequence of SEQ ID NO: 2, 4, 6, 8, or 10, or that encoded by SEQ ID NO: 1, 3, 5, 7, or 9.

In a preferred embodiment, the pharmaceutical composition comprises a protein having GM-CSF activity comprising an amino acid sequence with at least about 90% identity to SEQ ID NO: 12 or 13 or at least about 90% similarity to SEQ ID NO: 12 or 13. In another preferred embodiment, the targeting group is Vitamin D. In another preferred embodiment, the scaffold moiety is poly(ethylene glycol). In a most preferred embodiment, the invention contemplates a pharmaceutical composition comprising a protein having GM-CSF activity comprising an amino acid sequence with at least about 90% identity to SEQ ID NO: 12 or 13 or at least about 90% similarity to SEQ ID NO: 12 or 13, a scaffold moiety that is poly(ethylene glycol), and a targeting group that is Vitamin D. In this embodiment, the targeting group increases the absorption, bioavailability, or the half-life of the therapeutic compound in circulation. In another most preferred embodiment, the invention contemplates a pharmaceutical composition comprising a protein having GM-CSF activity and the amino acid sequence of SEQ ID NO: 12 or 13 or that encoded by SEQ ID NO: 11.

In certain embodiments, the present invention provides carriers that include those of formula I:

B-L¹-S-L²-L³-C 1

Wherein:
B is a targeting group selected from vitamin D, a vitamin D analog, a vitamin D-related metabolite, an analog of a vitamin D related-metabolite, a peptide that binds DBP, an anti-DBP antibody, an anti-DBP antibody derivative, a nucleotide aptamer that binds DBP, or a small carbon-based molecule that binds DBP;
S is a scaffold moiety, comprising poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, polylactic acid, a water-soluble polymer, a small carbon chain linker, or an additional therapeutic moiety;
C is an amine-reactive group, a thiol-reactive group, a maleimide group, a thiol group, a disulfide group, an aldehyde group, an NHS-ester group, a 4-nitrophenyl ester, an acylimidazole, a haloacetyl group, an iodoacetyl group, a bromoacetyl group, a SMCC group, a sulfo SMCC group, a carbodiimide group and bifunctional cross-linkers such as NHS-Maleimido or combinations thereof,
L¹ and L² are linkers independently selected from —(CH₂)ₙ—, —C(O)NH—, —HNC(O)—, —C(O)O—, —OC(O)—, —O—, —S—S—, —S—S—, —S(O)—, —S(O)₂— and —NH—.
L³ is —(CH₂)ₒ—;
n is an integer from 0-3; and
is an integer from 0-3.

In certain embodiments, the present invention provides a method for producing a carrier of formula I:

B-L¹-S-L²-L³-C I

comprising the step of reacting a compound of formula Ia:

B—COOH Ia

with a compound of formula Ib:

H₂N—S-L²-L³-C Ib

In the presence of an amide coupling agent,

Wherein B, S, C, L² and L³ are defined as above and L¹ is —C(O)NH—.

In certain other embodiments, the present invention provides a method for producing a carrier of formula I:

B-L¹-S-L²-L³-C I

comprising the step of reacting a compound of formula Ia:

B—COOH Ia

with a compound of formula Ic:

H₂N—S-L²-L³-COOR¹ Ic

In the presence of an amide coupling agent,

Hydrolyzing an ester to a carboxylic acid and,

Converting a carboxylic acid to an active ester,

Wherein B, S, L², L³ and n and o are defined as above,

L¹ is —C(O)NH— and,

R¹ is C₁-C₆ alkyl.

The invention provides a method of treating a patient in need of a therapeutic compound, comprising administering an effective amount of one or more of the pharmaceutical compositions described herein. Exemplary therapeutic compounds include small molecules, chemical entities, nucleic acids, nucleic acid derivatives, peptides, peptide derivatives, naturally-occurring proteins, non-naturally-occurring proteins, peptide-nucleic acids (PNA), stapled peptides, morpholinos, phosphorodiamidate morpholinos, antisense drugs, RNA-based silencing drugs, aptamers, glycoproteins, enzymes, hormones, cytokines, interferons, growth factors, blood coagulation factors, antibodies, antibody fragments, antibody derivatives, toxin-conjugated antibodies, metabolic effectors, analgesics, antipyretics, anti-inflammatory agents, antibiotics, anti-microbial agents, anti-viral agents, anti-fungal drugs, musculoskeletal drugs, cardiovascular drugs, renal drugs, pulmonary drugs, digestive disease drugs, hematologic drugs, urologic drugs, metabolism drugs, hepatic drugs, neurological drugs, anti-diabetes drugs, anti-cancer drugs, drugs for treating stomach conditions, drugs for treating colon conditions, drugs for treating skin conditions, and drugs for treating lymphatic conditions or G-CSF, G-CSF biosimilars, G-CSF interchangables or other compounds having G-CSF activity or GM-CSF, GM-CSF biosimilars, GM-CSF interchangeables or other compounds having GM-CSF activity whether singly or combined.

In preferred methods, the therapeutic compound is a protein having G-CSF activity comprising an amino acid sequence with at least a about 90% identity to SEQ ID NO: 2, 4, 6, 8, or 10, or about 90% similarity to SEQ ID NO: 2, 4, 6, 8, or 0. In other preferred methods, the targeting group is non-hormonal Vitamin D conjugated at Carbon 3 or the scaffold is poly(ethylene glycol).

In preferred methods, the therapeutic compound is a protein having GM-CSF activity comprising an amino acid sequence with at least about 90% identity to SEQ ID NO: 12 or 13 or about 90% similarity to SEQ ID NO: 12 or 13. In other preferred methods, the targeting group is non-hormonal Vitamin D conjugated at Carbon 3 or the scaffold is poly(ethylene glycol).

In other embodiments and methods, the pharmaceutical compositions of the invention are in pharmaceutically acceptable formulations. The pharmaceutical compositions may be delivered to patients by a transdermal, oral, parenteral, subcutaneous, intracutaneous, intravenous, intramuscular, intraarticular, intrasynovial, intrasternal, intrathecal, intralesional, intracranial injection, infusion, inhalation, ocular, topical, rectal, nasal, buccal, sublingual, vaginal, or implanted reservoir mode.

The invention provides the use of the disclosed pharmaceutical compositions for the manufacture of medicaments for the treatment of patients that need the medicaments.

The invention provides methods of manufacturing the pharmaceutical compositions disclosed herein comprising conjugating a targeting group and a drug into a carrier-drug compound utilizing coupling groups. The coupling groups may be amine-reactive coupling groups, maleimide coupling groups, cysteine coupling groups, aldehyde coupling groups, or thiol-reactive coupling groups. Maleimide is a useful coupling group for use in coupling to sulfhydryl groups such as on a free cysteine residue that can be site-specifically engineered into a peptide or protein in a desired position. Other coupling groups such as NHS—that target amine groups or aldehyde that can be used to site specifically attach to the N-terminus of a therapeutic compound are well known to those skilled in the art. Other more specialized coupling groups are contemplated and could be substituted by one skilled in the art.

In some methods, the targeting group is vitamin D, a vitamin D analog, a vitamin D-related metabolite, an analog of a vitamin D-related metabolite, a peptide that binds DBP, an anti-DBP antibody, an anti-DBP antibody derivative, a nucleotide aptamer that binds DBP, or a small carbon-based molecule that binds DBP.

In other embodiments, methods of manufacturing pharmaceutical compositions further comprise conjugating a scaffold moiety to the targeting group or drug. The scaffold moiety may be poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, or an additional therapeutic compound.

### BRIEF DESCRIPTION OF THE DRAWINGS

FIG. 1 is schematic diagram showing the general structure of a carrier coupled to a drug. The carrier comprises a targeting group, a scaffold, and optionally, a coupling group.

FIG. 2 is a schematic of hematopoiesis from the multipotential hematopoietic stem cell to fully differentiated cell types. Principal cytokines that determine differentiation patterns in red. Epo, Erythropoietin; FLT-3 ligand, FMS-like tyrosine kinase 3 ligand; G-CSF, Granulocyte-colony stimulating factor; GM-CSF, Granulocyte Macrophage-colony stimulating factor; IL, Interleukin; M-CSF, Macrophage-colony stimulating factor; SCF, Stem Cell Factor; SDF-1, Stromal cell-derived factor-1; TGFβ, Transforming growth factor beta; TNFα, Tumour necrosis factor-alpha; Tpo, Thrombopoietin; B.

FIG. 3 is a schematic showing stages of granulopoiesis from myeloblast to the mature granulocyte. During neutrophil maturation, which is driven primarily by G-CSF, granulocytic cells change shape, acquire primary and specific granules, and undergo nuclear condensation.

FIG. 4 is a reaction scheme drawing showing the chemical structures and syntheses used to generate a carrier, a Vitamin D₃-PEG-Maleimide adduct. The carrier was generated by conjugating 1) a Vitamin D analog (the targeting group), 2) a PEG scaffold, and 3) a maleimide coupling group.

FIG. 5 is a reaction scheme drawing showing the chemical structures and syntheses used to generate another carrier, a Vitamin D₃-PEG-NHS adduct. The carrier was generated by conjugating 1) a Vitamin D analog (the targeting group), 2) a PEG scaffold, and 3) an NHS coupling group.

FIG. 6 is a reaction scheme drawing showing the chemical structure and syntheses used to generate a carrier, a Vitamin D-(3)-PEG2k-aldehyde adduct. The carrier was generated by conjugating 1) a vitamin D analog, 2) a PEG scaffold, and 3) an aldehyde coupling group.

FIG. 7 is a reaction scheme drawing showing the chemical structure and syntheses used to generate a carrier, a Vitamin D-(3)-PEG2k-maleimide adduct. The carrier was generated by conjugating 1) a vitamin D analog, 2) a PEG scaffold, and 3) a maleimide coupling group.

FIG. 8 is a reaction scheme drawing showing the chemical structure and syntheses used to generate a carrier, a Vitamin D-(3)-PEG1.3k-NHS adduct. The carrier was generated by conjugating 1) a vitamin D analog, 2) a PEG scaffold, and 3) an NHS coupling group.

FIG. 9 is a reaction scheme drawing showing the chemical structure and syntheses used to generate GCSF-PEG₂₄-VitD.

FIG. 10 is a representation of SDS-PAGE analysis of the GSCF-PEG₂₄-VitD reaction. Lane a: See protein MW markers; Lane b: GSCF-PEG₂₄-VitD reaction; Lane c: G-CSF.

FIG. 11 is a reaction scheme drawing showing the chemical structure and syntheses used to generate GMCSF-PEG₂₄-VitD.

FIGS. 12A-C show nucleic acid and amino acid sequences for G-CSF (SEQ ID Nos: 1-10, respectively), GM-CSF (SEQ ID Nos: 11-13, respectively), DBP (SEQ ID Nos: 14 and 15, respectively), and amino acid sequence of PTH-C(SEQ ID NO: 16) and amino acid sequences of C-PTH (SEQ ID NO: 17).

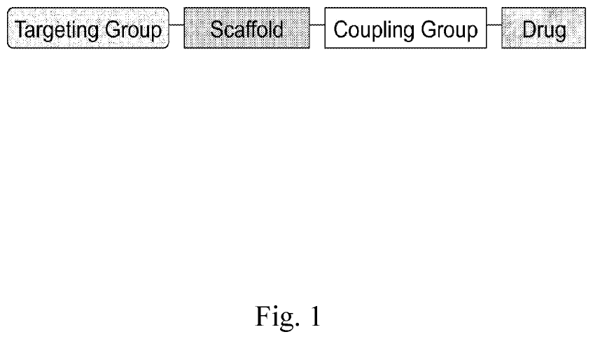

### DETAILED DESCRIPTION OF THE INVENTION

A description of preferred embodiments of the invention follows.

The invention relates to improving the potency, absorption or pharmacokinetic properties of therapeutic compounds. The addition of poly(ethylene glycol) or (PEG) is a known method of increasing the half-life of some compounds by reducing kidney clearance, reducing aggregation, and diminishing potentially unwanted immune recognition (Jain, Crit. Rev. Ther. Drug Carrier Syst. 25:403-447 (2008)). The PEG is typically used at a considerably large size (20-40 kDa) to maximize the half-life in circulation. This can be accomplished by using either a single large PEG or multiple smaller PEGs attached to the compound. (Clark et al. J. Biol. Chem. 271:21969-21977 (1996); Fishburn, J. Pharm. Sci. 97:4167-4183 (2008)).

There are number of biosimilars and interchangeables (e.g., variants, homologues and/or analogs) to G-CSF. The compound Granulocyte Colony Stimulating Factor (“G-CSF”) and biosimilars thereto are in wide-spread use or proposed use to correct neutropenia following chemotherapy treatment of various cancers. G-CSF suffers therapeutically from short half-life and low bioavailability and must be administered one or more days after the chemotherapy session requiring the patient to return to a medical provider for a subcutaneous injection of G-CSF. Also known as “filgrastim”, a commercial example of this form of recombinant human G-CSF is Neupogen made by Amgen.

The addition of poly(ethylene glycol) or (PEG) is a known method of increasing the half-life of some compounds by reducing kidney clearance, reducing aggregation, and diminishing potentially unwanted immune recognition (Jain, Crit. Rev. Ther. Drug Carrier Syst. 25:403-447 (2008)). The PEG is typically used at a considerably large size (20-40 kDa) to maximize the half-life in circulation. This can be accomplished by using either a single large PEG or multiple smaller PEGs attached to the compound. (Clark et al. J. Biol. Chem. 271:21969-21977 (1996); Fishburn, J. Pharm. Sci. 97:4167-4183 (2008)).

More recently, compositions of G-CSF and biosimilars thereto and a coating of Polyethylene Glycol (“PEG”) known as “PegG-CSF” have become widely used or proposed for use to correct neutropenia following chemotherapy.

PegG-CSF's are purported to have better half-life than G-CSF. However, PegG-CSF suffers from erratic half-life, poor bioavailability and must be administered one or more days after the chemotherapy session requiring the patient to return to a medical provider for a subcutaneous injection of PegG-CSF. One version of PegG-CSF has attempted to overcome the need for a return visit to a medical provider by providing an automated injector to be affixed to the patient's body at the time of chemotherapy to automatically give the PegG-CSF injection the next day. Also known a “pegfilgrastim”, a version of PegG-CSF is Neulasta made by Amgen. Another such is made by Mylan and sold under the brand name Fulphila. Amgen is a supplier of an automatic PegG-CSF pump known as the OnPro.

GM-CSF has been identified for numerous disease state including treatment after bone marrow transplant failure, after engraftment delay and after stem-cell transplant, as an immune stimulant in tumor cell and dendritic cell vaccines, to increase antibody-dependent cellular cytotoxicity, management of renal cell carcinoma and malignant melanoma, for its anti-inflammatory properties and in combination with cytotoxic or other targeted therapies including with G-CSF. Pegalation and glycosylation have been attempted to extend the half life of GM-CSF with little impact. No extended half life version of GM-CSF is known. Recombinant human GM-CSF is available from Amgen under the tradename Leukine.

The present invention provides a new chemical entity which conjugates G-CSF (or a biosimilar or interchangeable thereof) to a metabolite of Vitamin D. This new chemical entity is DVitylated G-CSF.

DVitylation provides greatly extended half-life to many therapeutics. DVityation also significantly improves bioavailability and is expected to enable dosing of DVitylated G-CSF by a patch or other simple, convenient means of administration.

The present invention also provides a new chemical entity which conjugates GM-CSF (or a biosimilar or interchangeable thereof) to a metabolite of Vitamin D. This new chemical entity is DVitylated GM-CSF.

DVitylation provides greatly extended half-life to many therapeutics. DVityation also significantly improves bioavailability and is expected to enable dosing of DVitylated GM-CSF by a patch or other simple, convenient means of administration.

The invention contemplates the use of DVitylated G-CSF or DVitylated GM-CSF singly or in combination with one another or other therapies.

The invention provides carrier molecules that are covalently attached to, fused to or formulated with therapeutic proteins, peptides, nucleic acids, small molecules including G-CSF, biosimilars and interchangeables of G-CSF and compounds having G-CSF activity and GM-CSF, biosimilars and interchangeables of GM-CSF and compounds having GM-CSF activity for the purpose of improving the potency, absorption, bioavailability, circulating half-life or pharmacokinetic properties of the therapeutic compounds. In certain embodiments, the carriers comprise a targeting group, a scaffold, and a coupling group. In other embodiments, the carriers lack a scaffold, which acts, among other things, as a “spacer” between the targeting group and the therapeutic compound.

The invention provides carrier-drug conjugates comprising targeting groups that are non-hormonal vitamin D, vitamin D analogs, or vitamin D metabolites. Examples include vitamin D-based molecules that are not hydroxylated at the carbon 1 (C1) position. The carriers are linked to therapeutic compounds at the carbon 25 (C25), at the carbon 3 (C3) position or other cabob position on the carrier. As disclosed herein, carrier groups are surprisingly effective when non-hormonal vitamin D forms are used and the therapeutic compound is linked to the Carbon 3 position. While not wishing to be bound by theory, it is believed that the hormonal forms of vitamin D are not appropriate for the carriers described herein because they can be toxic due to the induction of hypercalcemia. Also, because the hormonal forms bind the vitamin D receptor in cells, they may improperly target the carrier-drug conjugates to undesired cells or tissues. In contrast, non-hormonal vitamin D forms bind the Vitamin D Binding Protein (DBP) and remain in circulation longer.

The carrier molecules are attached to the therapeutic compounds using chemistries described herein, described in WO2013172967, incorporated herein in its entirety, or that are otherwise known in the art. The carriers improve the potency, absorption, bioavailability, circulating half-life or pharmacokinetic properties of the therapeutic compounds. In certain embodiments, the carriers further comprise what will be described herein as a “scaffold” that acts, among other things, as a non-releasable “spacer” between the targeting group and the therapeutic compound. In other embodiments, the carriers lack a scaffold. The carriers are designed to be suitable for use in humans and animals. The carriers serve the purpose of improving the pharmacokinetic properties of a biological or chemical entity that is coupled, conjugated, or fused to the carrier. This occurs through the interaction of the targeting group with DBP. DBP can actively transport molecules quickly and effectively from the site of administration to the circulating plasma, thereby reducing exposure of the drug to degradative enzymes. The carriers, by binding to DBP, also improve the circulating half-life of the drug. This increases the potency and therapeutic efficacy of the drug by preventing kidney filtration and other elimination processes.

The impact on patient health of this new class of therapies will be profound. Many previously unusable therapies for serious conditions such as ghrelin for cancer cachexia, apelin for pulmonary arterial hypertension, diabetes, and cardiac disease, and PTH for hypoparathyroidism could be realized by application of this invention. Improvements in other current therapies such as insulin and GLP1 for treating diabetes could have a big impact on patient health and convenience. A large number of diseases will benefit from treatment with drugs having extended half-life G-CSF activity or GM-CSF activity alone or in combination.

GM-CSF is more widely expressed than G-CSF and has different receptor expression than G-CSF. GM-CSF is the main CSF released by cells of the lung in response to inflammatory cytokines. A large number of disease states may benefit from GM-CSF-based treatments. As described by the FDA label for Leukine (sargramostim), the recombinant version of GM-CSF sold by Genzyme, these include myeloid reconstitution after autologous or allogenic bone marrow transplantation, chemotherapy induced neutropenia and as countermeasure for radiation induced bone marrow myelogenesis. Sargramostim is only available as a liquid formulation with benzyl alcohol for intravenous administration. Benzyl alcohol is toxic to babies. Sargramostim exhibits very low half-life and poor bioavailability. Each of these negative features are elevated by the invention described here.

The therapeutic and potential therapeutic use of GM-CSF has also been described as a treatment after bone marrow transplant failure, after engraftment delay and after stem-cell transplant, as an immune stimulant in tumor cell and dendritic cell vaccines, to increase antibody-dependent cellular cytotoxicity, management of renal cell carcinoma and malignant melanoma, for its anti-inflammatory properties and in combination with cytotoxic or other targeted therapies including with G-CSF. Arellano, et al, Clinical Uses of GM-CSF, a critical appraisal and update, Biologics, 2008, March; 2(1) 13-27, 6. GM-CSF has also been described as having ant-bacterial, anti-fungal and anti-viral properties. Damiani, G, et al., Recombinant human granulocyte macrophage-colony stimulating factor expressed in yeast (sargramostim), Clin Immunol. 2020 January; 210:108292.

In describing and claiming one or more embodiments of the present invention, the following terminology will be used in accordance with the definitions described below.

The carriers are designed to be suitable for use in humans and animals. The carriers serve the purpose of improving the pharmacokinetic properties of a biological or chemical entity that is coupled to, conjugated to, fused to, or formulated with the carrier. This occurs through the interaction of the targeting group with vitamin D binding protein (DBP), which can actively transport molecules quickly and effectively from the site of administration to the circulating plasma, thereby reducing exposure of the drug to degradative enzymes. The carriers, by binding to DBP, also improve the circulating half-life of the drug, thus increasing the potency and therapeutic efficacy of the drug by preventing kidney filtration. Methods for conjugating the carrier to therapeutic compounds described herein are known in the art.

By way of example, conjugation using the coupling groups of the invention may be carried out using the compositions and methods described in WO93/012145 (Atassi et al.) and U.S. Pat. No. 7,803,777 (Defrees et al.), each of which are incorporated by reference herein in their entirety.

In describing and claiming one or more embodiments of the present invention, the following terminology will be used in accordance with the definitions described below.

The term “absorption” is the movement of a drug into the bloodstream. A drug needs to be introduced via some route of administration (e.g. oral, topical or dermal) or in a specific dosage form such as a tablet, capsule or liquid. Intravenous therapy, intramuscular injection, and enteral nutrition provide less variability in absorption and bioavailability is often near 100%. The fastest route of absorption is inhalation. A convenient route of administration is transdermally by a “patch” or time-release “patch”.

An “antagonist” refers to a molecule capable of neutralizing, blocking, inhibiting, abrogating, reducing or interfering with the activities of a particular or specified protein, including its binding to one or more receptors in the case of a ligand, or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of proteins, hormones, or other bioactive molecules. Antagonists may be fusion proteins, receptor molecules, antisense molecules, aptamers, ribozymes, or derivatives that bind specifically to the proteins, hormones, or other bioactive molecules and thereby sequester its binding to its target.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

“Aptamers” are nucleic acid-based compounds that have been selected to bind a specific target. An example of an aptamer-based therapeutic compound can be found in WO07/035922, incorporated by reference herein in its entirety.

The term “bioavailability” refers to the fraction of an administered dose of unchanged drug that reaches the systemic circulation, one of the principal pharmacokinetic properties of drugs. When a medication is administered intravenously, its bioavailability is 100%. When a medication is administered via other routes (such as orally or transdermally), its bioavailability generally decreases (due to incomplete absorption and first-pass metabolism) or may vary from patient to patient. Bioavailability is an important parameter in pharmacokinetics that is considered when calculating dosages for non-intravenous routes of administration.

“Biosimilar” means that the biological product is highly similar to an FDA, EMA or other approving agency, approved biological product, known as a reference product, and that there are no clinically meaningful differences between the biosimilar product and the reference product., or the product otherwise qualifies as a biosimilar or interchangeable product to the invention by the regulations and/or agency in effect at the time.

The term “interchangeable” or “interchangeability”, in reference to a biological product that is shown to meet standards approved by the pertinent regulatory authority such as the FDA or EMA, means that the biological product may be substituted for the reference product without the intervention of the health care provider who prescribed the reference product.

As used herein, ‘biosimilar” includes products and methods which are interchangeable with G-CSF or GM-CSF singly or in combination.

Known biosimilars to G-CSF include:

Note: The originator product, Amgen's Neupogen (filgrastim), was approved by the US Food and Drug Administration (FDA) in February 1991. Filgrastim differs slightly from naturally occurring (wild) G-CSF.

TABLE 1
Biosimilars and non-originator biologicals* of filgrastim approved or in
development

| Company name, Country | Product name | Stage of development |
|---|---|---|
| Adello Biologies | — | Accepted for review by FDA in September |
| | | 2017 [2] |
| Apotex (Apobiologix), Canada | Grastofil | Biosimilar approved in the EU in October 2013 |
| | | for neutropenia [3]. Application for approval |
| | | submitted to US FDA via abbreviated |
| | | biosimilars pathway in February 2015 [4]. |
| Aryogen Biopharma, Iran* | TinaGrast | ‘Biogeneric’ marketed in Iran |
| Biocon, India* | Nufil | ‘Similar biologic’ marketed in India [5] |
| Biosidus, Argentina* | Granulostim/ | Medicamento biologico similar approved in |
| | Neutromax | Argentina |
| Cadila Pharmaceutical, India* | Filgrastim | ‘Similar biologic’ approved in India in October |
| | | 2013 [5] |
| Claris Life Sciences, India* | Fegrast | ‘Similar biologic’ marketed in India [5] |
| CT Arzneimittel, Germany | Biograstim | Biosimilar marketed in EU, where it was |
| | | approved in September 2008 for cancer, |
| | | haematopoietic stem cell transplantation, and |
| | | neutropenia [3]. |
| Dr Reddy’s Laboratories, | Grafeel | ‘Similar biologic’ marketed in India [5] |
| India* | | |
| Eurofarma | Fiprima | Follow-on biological approved in Brazil in |
| Laboratorios, Brazil | | October 2015 [6] |
| Hexal, Germany | Filgrastim | Biosimilar marketed in EU, where it was |
| | Hexal (EP2006) | approved in February 2009 for cancer, |
| | | haematopoietic stem cell transplantation and |
| | | neutropenia [3]. |
| Hospira (Pfizer), USA | Nivestim (EU)/ | Biosimilar marketed in EU, where it was |
| | Nivestym (US) | approved in June 2010 for cancer, |
| | | haematopoietic stem cell transplantation and |
| | | neutropenia [3]. Approved by FDA in July |
| | | 2018 [7]. |
| Intas Biopharmaceuticals, | Neukine | ‘Similar biologic’ approved in India in July |
| India* | | 2004 [5] |
| Gennova Biopharmaceuticals | Emgrast | ‘Similar biologic’ approved in India in March |
| (Emcure), India* | | 2010 [5] |
| Lupin, India* | Filgrastim | ‘Similar biologic’ approved in India in March |
| | | 2013 [4] |
| Merck (MSD) | MK-4214 | Phase III trial in breast cancer prematurely |
| | | ended |
| Nanogen Pharmaceutical, | Ficocyte | ‘Product’ marketed in Vietnam |
| Vietnam* | | |
| Pooyesh Darou | PDGRASTIM | ‘Biogeneric’ marketed in Iran |
| Biopharmaceutical, Iran* | | |
| Ratiopharm, Germany | Ratiograstim | Biosimilar marketed in EU, where it was |
| | | approved in September 2008 for cancer, |
| | | haematopoietic stem cell transplantation and |
| | | neutropenia [3]. |
| Reliance Life Sciences, India* | Religrast | ‘Similar biologic’ approved in India in 2008 |
| | | [5] |
| Sandoz, Switzerland | Zarzio (EU)/ | Biosimilar marketed in the EU where it was |
| | Zarxio | approved in February 2009 for cancer, |
| | (USA) (EP2006) | haematopoietic stem cell transplantation and |
| | | neutropenia [3]. Received Japanese approval in |
| | | March 2014 [8]. Approved by FDA in March |
| | | 2015 [9] |
| Stada Arzneimittel, Germany | Grastofil | Biosimilar in-licensed from Apotex in October |
| | | 2013. Marketing expected to start in all the EU |
| | | countries in 2014 [10] |
| Tanvex BioPharma, Taiwan | TX-01 | Biosimilar application for approval submitted |
| | | to US FDA in October 2018 [11] |
| Teva Pharmaceutical | Tevagrastim | Biosimilar marketed in the EU, where it was |
| Industries, Israel | | approved in September 2008 for cancer, |
| | | haematopoietic stem cell transplantation and |
| | | neutropenia [3]. Non-originator biological |
| | | approved in South Africa in November 2017 |
| | | [12] |
| USV, India* | Filgrastim | ‘Similar biologic’ approved in India in June |
| | | 2013 [5] |

Known biosimilars of GM-CSF include Leukine (also known as Sargramostim) made by Genzyme.

“Carriers” are compounds that can be conjugated to, fused to, coupled to or formulated with therapeutic compounds to improve the absorption, half-life, bioavailability, pharmacokinetic or pharmacodynamic properties of the drugs. They comprise a targeting group, a coupling group, and optionally, a scaffold moiety. In some embodiments, carriers may carry a therapeutic compound from the site of subcutaneous injection into circulation as well as carry the therapeutic compound in circulation for an extended period of time.

An “effective amount” refers to an amount of therapeutic compound that is effective, at dosages and for periods of time necessary, to achieve the desired therapeutic or prophylactic result. A “therapeutically effective amount” of a therapeutic compound may vary according to factors such as the disease state, age, sex, and weight of the individual, and the ability of the antibody to elicit a desired response in the individual. A therapeutically effective amount may be measured, for example, by improved survival rate, more rapid recovery, or amelioration, improvement or elimination of symptoms, or other acceptable biomarkers or surrogate markers. A therapeutically effective amount is also one in which any toxic or detrimental effects of the therapeutic compound are outweighed by the therapeutically beneficial effects. A “prophylactically effective amount” refers to an amount of therapeutic compound that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Half-life” is a scientific term known in the art that refers to the amount of time that elapses when half of the quantity of a test molecule is no longer detected. An in vivo half-life refers to the time elapsed when half of the test molecule is no longer detectable in circulating serum or tissues of a human or animal.

“Having G-CSF activity” means G-CSF, natural variants of G-CSF, manufacturing variants of G-CSF including recombinant, biosimilars of G-CSF, compounds or formulations which are interchangeable with G-CSF, PegG-CSF and compounds or formulations deemed by a regulatory or statutory body to be biosimilar, interchangeable or otherwise usable in place of G-CSF or PegG-CSF.

“Having GM-CSF activity” means GM-CSF, natural variants of GM-CSF, manufacturing variants of GM-CSF including recombinant, biosimilars of GM-CSF, compounds or formulations which are interchangeable with GM-CSF, PegGM-CSF and compounds or formulations deemed by a regulatory or statutory body to be biosimilar, interchangeable or otherwise usable in place of GM-CSF or PegG-CSF.

A “hormone” is a biological or chemical messenger from one cell (or group of cells) to another cell that has signaling capability. As described herein, hormones for use in the invention may be peptides, steroids, pheromones, interleukins, lymphokines, cytokines, or members of other hormone classes known in the art.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a preferred embodiment, homologous or derivative sequences share at least an 80 or 85 percent sequence identity. In a more preferred embodiment, homologous or derivative sequences share at least about 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art. Homologs are biosimilars as can be “analogs”.

“Hybridization” generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers, (1995).

An “individual,” “subject” or “patient” is a vertebrate. In certain embodiments, the vertebrate is a mammal. Mammals include, but are not limited to, primates (including human and non-human primates) and rodents (e.g., mice, hamsters, guinea pigs, and rats). In certain embodiments, a mammal is a human. A “control subject” refers to a healthy subject who has not been diagnosed as having a disease, dysfunction, or condition that has been identified in an individual, subject, or patient. A control subject does not suffer from any sign or symptom associated with the disease, dysfunction, or condition.

A “medicament” is an active drug that has been manufactured for the treatment of a disease, disorder, or condition.

“Morpholinos” are synthetic molecules that are non-natural variants of natural nucleic acids that utilize a phosphorodiamidate linkage, described in U.S. Pat. No. 8,076,476, incorporated by reference herein in its entirety.

“Nucleic acids” are any of a group of macromolecules, either DNA, RNA, or variants thereof, that carry genetic information that may direct cellular functions. Nucleic acids may have enzyme-like activity (for instance ribozymes) or may be used to inhibit gene expression in a subject (for instance RNAi). The nucleic acids used in the inventions described herein may be single-stranded, double-stranded, linear or circular. The inventions further incorporate the use of nucleic acid variants including, but not limited to, aptamers, PNA, Morpholino, or other non-natural variants of nucleic acids. By way of example, nucleic acids useful for the invention are described in U.S. Pat. No. 8,076,476, incorporated by reference herein in its entirety.

“Patient response” or “response” can be assessed using any endpoint indicating a benefit to the patient, including, without limitation, (1) inhibition, to some extent, of disease progression, including slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

As used herein, the term “peptide” is any peptide comprising two or more amino acids. The term peptide includes short peptides (e.g., peptides comprising between 2-14 amino acids), medium length peptides (15-50) or long chain peptides (e.g., proteins). The terms peptide, medium length peptide and protein may be used interchangeably herein. As used herein, the term “peptide” is interpreted to mean a polymer composed of amino acid residues, related naturally occurring structural variants, and synthetic non-naturally occurring analogs thereof linked via peptide bonds, related naturally-occurring structural variants, and synthetic non-naturally occurring analogs thereof. Synthetic peptides can be synthesized, for example, using an automated peptide synthesizer. Peptides can also be synthesized by other means such as by cells, bacteria, yeast or other living organisms. Peptides may contain amino acids other than the 20 gene-encoded amino acids. Peptides include those modified either by natural processes, such as processing and other post-translational modifications, but also by chemical modification techniques. Such modifications are well described in basic texts and in more detailed monographs, and are well-known to those of skill in the art. Modifications occur anywhere in a peptide, including the peptide backbone, the amino acid side-chains, and the amino or carboxyl termini. G-CSF and compounds having G-CSF activity are peptides.

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of specific inhibitor, for example, carbohydrates, such as glucose, sucrose or dextrans, antioxidants such as ascorbic acid or glutathione, chelating agents, low molecular weight proteins or other stabilizers or excipients.

The term “pharmacokinetics” is defined as the time course of the absorption, distribution, metabolism, and excretion of a therapeutic compound. Improved “pharmacokinetic properties” are defined as: improving one or more of the pharmacokinetic properties as desired for a particular therapeutic compound. Examples include but are not limited to: reducing elimination through metabolism or secretion, increasing drug absorption, increasing half-life, and/or increasing bioavailability.

“PNA” refers to peptide nucleic acids with a chemical structure similar to DNA or RNA. Peptide bonds are used to link the nucleotides or nucleosides together.

“Scaffolds” are molecules to which other molecules can be covalently or or non-covalently attached or formulated. The scaffolds of the invention may act as “spacers” or “linkers” between the targeting group and the drug. Scaffolds may also contain a reactive linker or may have beneficial therapeutic properties in addition to the drug. Thus, the scaffolds of the invention may be, for example, PEG, serum albumin, thioredoxin, an immunoglobulin, a modifying group that contains a reactive linker, a water-soluble polymer, or a therapeutic compound.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 l/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

The “therapeutic compounds” disclosed herein refer to G-CSF and compounds having G-CSF activity as well as small molecules, chemical entities, nucleic acids, nucleic acid derivatives, peptides, peptide derivatives, naturally-occurring proteins, non-naturally-occurring proteins, glycoproteins, and steroids that are administered to subjects to treat a diseases or dysfunctions or to otherwise affect the health of individuals. Non-limiting examples of therapeutic compounds include polypeptides such as enzymes, hormones, cytokines, antibodies or antibody fragments, antibody derivatives, drugs that affect metabolic function, as well as organic compounds such as analgesics, antipyretics, anti-inflammatory agents, antibiotics, anti-viral compounds, anti-fungal compounds, cardiovascular drugs, drugs that affect renal function, electrolyte metabolism, drugs that act on the central nervous system, chemotherapeutic compounds, receptor agonists and receptor antagonists. Therapeutic compounds include, for example, extracellular molecules such as serum factors including, but not limited to, plasma proteins such as serum albumin, immunoglobulins, apolipoproteins or transferrin, or proteins found on the surface of erythrocytes or lymphocytes. Thus, exemplary therapeutic compounds include small molecules, chemical entities, nucleic acids, nucleic acid derivatives, peptides, peptide derivatives, naturally-occurring proteins, non-naturally-occurring proteins, peptide-nucleic acids (PNA), stapled peptides, phosphorodiamidate morpholinos, antisense drugs, RNA-based silencing drugs, aptamers, glycoproteins, enzymes, hormones, cytokines, interferons, growth factors, blood coagulation factors, antibodies, antibody fragments, antibody derivatives, toxin-conjugated antibodies, metabolic effectors, analgesics, antipyretics, anti-inflammatory agents, antibiotics, anti-microbial agents, anti-viral agents, anti-fungal drugs, musculoskeletal drugs, cardiovascular drugs, renal drugs, pulmonary drugs, digestive disease drugs, hematologic drugs, urologic drugs, metabolism drugs, hepatic drugs, neurological drugs, anti-diabetes drugs, anti-cancer drugs, drugs for treating stomach conditions, drugs for treating colon conditions, drugs for treating skin conditions, drugs for treating lymphatic conditions or G-CSF or others having G-CSF activity The term “therapeutic compound” as used herein has essentially the same meaning as the terms “drug” or “therapeutic agent.”

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

A “vitamin” is a recognized term in the art and is defined as a fat-soluble or water-soluble organic substance essential in minute amounts for normal growth and activity of the body and is obtained naturally from plant and animal foods or supplements.

“Vitamin D” is a group of fat-soluble secosteroids. Several forms (vitamers) of vitamin D exist.

The two major forms are vitamin D₂ or ergocalciferol, and vitamin D₃ or cholecalciferol. Vitamin D without a subscript refers to either D₂ or D₃ or both. In humans, vitamin D can be ingested as cholecalciferol (vitamin D₃) or ergocalciferol (vitamin D₂). Additionally, humans can synthesize it from cholesterol when sun exposure is adequate. Cholecalciferol is modified in the liver or in vitro to 25-hydroxycholecalciferol (“25-hydroxy Vitamin D”). In the kidney or in vitro, 25-hydroxy vitamin D can be modified into the distinct hormonal form of 1, 25-hydroxy vitamin D.

“Vitamin D binding protein” or “DBP” is a naturally circulating serum protein found in all mammals that, among other activities, can bind to and transport vitamin D and its analogs to sites in the liver and kidney where the vitamin is modified to its active form, and it retains vitamin D in its various forms in circulation for, on average, 30 days in humans. A DBP protein sequence is disclosed in SEQ ID NO:14 and an exemplary nucleic acid sequence encoding the DBP protein sequence is disclosed in SEQ ID NO:15. DBP has multiple naturally-occurring isoforms. Exemplary isoforms are available in the public sequence databases (e.g. Accession Nos. NM-001204306.1, NM-001204307.1, NM-000583.3, BC036003.1, M12654.1, X03178.1, AK223458, P-001191235.1, NP-000574.2, AAA61704.1, AAD13872.1, NP-001191236.1, AAA19662.2, I54269, P02774.1, EAX05645.1, AAH57228.1, AAA52173.1, AAB29423.1, AAD14249.1, AAD14250.1, and BAD97178.1).

The invention contemplates the use of DBP variants and homologs that contain conservative or non-conservative amino acid substitutions that substantially retain DBP activity. DBP binding molecules or functional DBP variants may be identified using known techniques and characterized using known methods (Bouillon et al., J Bone Miner Res. 6(10):1051-7 (1991), Teegarden et. al., Anal. Biochemistry 199(2):293-299 (1991), McLeod et al, J Biol Chem. 264(2):1260-7 (1989), Revelle et al., J. Steroid Biochem. 22:469-474 (1985)) The foregoing references are incorporated by reference herein in their entirety.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like.

The invention provides effective routes for administration of proteins, peptides, other biologics, nucleic acids, and small molecule drugs including G-CSF and those having G-CSF activity and of GM-CSF and those having GM-CSF activity. The invention further provides effective routes of drug administration via transdermal, oral, parenteral, subcutaneous, intracutaneous, intravenous, intramuscular, intraarticular, intrasynovial, intrasternal, intrathecal, intralesional, intracranial injection, infusion, inhalation, ocular, topical, rectal, nasal, buccal, sublingual, vaginal, or implanted reservoir modes.

In addition, the inventions described herein provide compositions and methods for maintaining target binding activity, i.e. pharmacodynamics (PD), for therapeutic compounds. It further provides compositions and methods for improving the pharmacokinetic (PK) profiles of therapeutic compounds as described herein. The invention further provides compositions and methods for improved drug absorption profiles as compared to the drug absorption profiles for the drugs using the same routes of administration or different routes of administration but without the inventions described herein. The invention further provides compositions and methods for improved drug bioavailability profiles as compared to the drug bioavailability profiles for the drugs using the same routes of administration or different routes of administration but without the inventions described herein. The invention further provides compositions and methods for improved drug half-life profiles as compared to the drug half-life profiles for the drugs using the same routes of administration or different routes of administration but without the inventions described herein.

The invention also provides alternative routes of drug administration that are more cost-effective and favorable to the patients when compared to the drugs without the inventions described herein.

The invention provides compositions and methods for using molecules that serve as carriers that can be conjugated to, fused to, or formulated with active therapeutic compounds for the purpose of improving the absorption, half-life, bioavailability, or pharmacokinetic properties of the drugs. The carriers have the properties of binding to the body's natural DBP. One aspect of the invention provides use of the natural DBP to transport the carrier-drug complex from the site of administration to the circulating serum. Another aspect of the invention is the use of the natural DBP to retain a drug in circulation for an extended period of time. This can prevent its excretion from the body and increase the exposure of the therapeutic compound in the body to achieve a longer lasting therapeutic effect. In another aspect of the invention, a smaller dose of drug is required when conjugated to, fused to or formulated with the carrier, when compared to the unconjugated, unfused or unformulated drug. Another aspect of the invention is the use of a carrier to replace the function of a much larger PEG compound when coupled to a therapeutic compound. This can improve the pharmacokinetic profile and efficacy of the conjugated, fused or formulated compound.

The invention provides a carrier molecule that is preferably composed of one or more parts or components. In one embodiment, the carrier comprises a targeting group and a coupling group for attaching the targeting group to the therapeutic compound. In another embodiment, the carrier comprises a scaffold moiety that is linked to the targeting group and the therapeutic compound. The targeting group is vitamin D, a vitamin D analog, a vitamin D-related metabolite, a vitamin D-related metabolite analog, or another molecule that can bind to or interact with the vitamin D binding protein (DBP). In one embodiment, the targeting group is an antibody or antibody derivative, a peptide designed to bind DBP or a fragment thereof, a peptide derived from a phage display or other peptide library selected against DBP or a fragment thereof, a nucleotide aptamer that binds DBP, a small molecule designed to bind DBP or derived from a chemical library selected against DBP, or a fragment thereof.

“Vitamin D binding protein” or “DBP” is a naturally circulating serum protein found in all mammals that, among other activities, can bind to and transport vitamin D and its analogs to sites in the liver and kidney where the vitamin is modified to its active form, and it retains vitamin D in its various forms in circulation for, on average, 30 days in humans. A DBP protein sequence is disclosed in SEQ ID NO:14 and an exemplary nucleic acid sequence encoding the DBP protein sequence is disclosed in SEQ ID NO: 15. DBP has multiple naturally-occurring isoforms. Exemplary isoforms are available in the public sequence databases (e.g. Accession Nos. NM_001204306.1, NM_001204307.1, NM_000583.3, BC036003.1, M12654.1, X03178.1, AK223458, P_001191235.1, NP000574.2, AAA61704.1, AAD13872.1, NP_001191236.1, AAA19662.2, 154269, P02774.1, EAX05645.1, AAH57228.1, AAA52173.1, AAB29423.1, AAD14249.1, AAD14250.1, and BAD97178.1).

The invention contemplates non-hormonal vitamin D conjugates that bind DBP or functional DBP variants and homologs that contain conservative or non-conservative amino acid substitutions that substantially retain DBP activity. DBP binding molecules or functional DBP variants may be identified using known techniques and characterized using known methods (Bouillon et al., J Bone Miner Res. 6(10):1051-7 (1991), Teegarden et. al., Anal. Biochemistry 199(2):293-299 (1991), McLeod et al, J Biol Chem. 264(2):1260-7 (1989), Revelle et al., J. Steroid Biochem. 22:469-474 (1985)). The foregoing references are incorporated by reference herein in their entirety.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like.

The invention provides effective routes for administration of proteins, peptides, other biologics, nucleic acids, small molecule drugs or G-CSF or compounds with G-CSF activity and GM-CSF or compounds with GM-CSF activity.

The invention further provides effective routes of drug administration via transdermal, oral, parenteral, subcutaneous, intracutaneous, intravenous, intramuscular, intraarticular, intrasynovial, intrasternal, intrathecal, intralesional, intracranial injection, infusion, inhalation, ocular, topical, rectal, nasal, buccal, sublingual, vaginal, or implanted reservoir modes.

In addition, the inventions described herein provide compositions and methods for maintaining target binding activity, i.e. pharmacodynamics (PD), for therapeutic compounds. It further provides compositions and methods for improving the pharmacokinetic (PK) profiles of therapeutic compounds as described herein. The invention further provides compositions and methods for improved drug absorption profiles as compared to the drug absorption profiles for the drugs using the same routes of administration or different routes of administration but without the inventions described herein. The invention further provides compositions and methods for improved drug bioavailability profiles as compared to the drug bioavailability profiles for the drugs using the same routes of administration or different routes of administration but without the carriers described herein. The invention further provides compositions and methods for improved drug half-life profiles as compared to the drug half-life profiles for the drugs using the same routes of administration or different routes of administration but without the inventions described herein. The invention also provides alternative routes of drug administration that are more cost-effective or favorable to the patients when compared to the drugs without the inventions described herein.

The non-hormonal vitamin D carriers disclosed herein may improve the absorption, half-life, bioavailability, or pharmacokinetic properties of the linked therapeutic compounds. While not wishing to be bound by theory, the carriers have the properties of binding to the body's natural DBP. DBP may transport the carrier-drug complex from the site of administration to the circulating serum. The vitamin D-DBP interaction may retain the therapeutic compounds in circulation for an extended period of time. This can prevent its excretion from the body and increase the exposure of the therapeutic compound in the body to achieve a longer lasting therapeutic effect. Additionally, a smaller dose of drug may be required when conjugated the carrier when compared to the unmodified form.

The therapeutic compound carrier conjugates of the invention typically have about 1, 2, 3, 4, 5, 6, 7, 8, 9, or 10 targeting groups individually attached to a therapeutic compound. The structure of each of the targeting groups attached to the therapeutic compound may be the same or different. In preferred embodiments, one or more targeting groups are stably or non-releasably attached to the therapeutic compound at the N-terminus, C-terminus, or other portion of a therapeutic protein. For example, a therapeutic compound carrier conjugate may comprise a targeting group attached to the N-terminus and additionally a targeting group attached to a lysine residue. In another embodiment, a therapeutic compound carrier conjugate has a targeting group attached to a therapeutic protein via a modification such as a sugar residue as part of a glycosylation site, or on an acylation site of a peptide or attached to a phosphorylation site or other natural or non-natural modifications that are familiar to one skilled in the art. Also contemplated are attachment sites using a combination of sites mentioned above. One preferred embodiment of the present invention comprises a targeting group that is attached to the therapeutic compound at one specific site on a therapeutic compound. In another preferred embodiment, the attachment site on a protein may be a cysteine, lysine, the N-terminus or C-terminus.

In another embodiment, the scaffold is a pharmaceutically acceptable carrier. In preferred embodiments, the scaffold is poly(ethylene glycol), polylysine, polyethyleneimine, poly(propyleneglycol), a peptide, serum albumin, thioredoxin, an immunoglobulin, an amino acid, a nucleic acid, a glycan, a modifying group that contain a reactive linker, a water-soluble polymer, a small carbon chain linker, or an additional therapeutic moiety. In one embodiment, water-soluble scaffold moieties have some detectable degree of solubility in water. Methods to detect and/or quantify water solubility are well known in the art. Exemplary water-soluble polymers include peptides, saccharides, poly(ethers), poly(amines), poly(carboxylic acids) and the like.

The therapeutic compound carrier conjugates of the invention typically have about 1, 2, 3, 4, 5, 6, 7, 8, 9, or 10 targeting groups individually attached to a therapeutic compound. In one embodiment, the carrier conjugate of the invention will comprise about 4 targeting groups individually attached to a therapeutic compound, or about 3 targeting groups individually attached to a therapeutic compound, or about 2 targeting groups individually attached to a therapeutic compound, or about 1 targeting group attached to a therapeutic compound. The structure of each of the targeting groups attached to the therapeutic compound may be the same or different. In a preferred embodiment, one or more targeting groups are stably attached to the therapeutic compound at the N-terminus of a therapeutic protein. In another preferred embodiment, one or more targeting groups are stably attached to the therapeutic protein at the C-terminus of a therapeutic protein. In other preferred embodiments, one or more targeting groups may be stably attached to other sites on the therapeutic protein. For example, a therapeutic compound carrier conjugate may comprise a targeting group attached to the N-terminus and additionally a targeting group attached to a lysine residue. In another embodiment, a therapeutic compound carrier conjugate has a targeting group attached to a therapeutic protein via a modification such as a sugar residue as part of a glycosylation site, or on an acylation site of a peptide or attached to a phosphorylation site or other natural or non-natural modifications that are familiar to one skilled in the art. Also contemplated are attachment sites using a combination of sites mentioned above. One preferred embodiment of the present invention comprises a targeting group that is attached to the therapeutic compound at one specific site on a therapeutic compound. In another preferred embodiment, the attachment site on a protein may be a cysteine, lysine, the N-terminus or C-terminus.

In another embodiment, the scaffold is a pharmaceutically acceptable carrier. In preferred embodiments, the scaffold is poly(ethylene glycol), polylysine, polyethyleneimine, poly(propyleneglycol), a peptide, serum albumin, thioredoxin, an immunoglobulin, an amino acid, a nucleic acid, a glycan, a modifying group that contain a reactive linker, a water-soluble polymer, a small carbon chain linker, or an additional therapeutic moiety.

In one embodiment, water-soluble scaffold moieties have some detectable degree of solubility in water. Methods to detect and/or quantify water solubility are well known in the art. Exemplary water-soluble polymers include peptides, saccharides, poly(ethers), poly(amines), poly(carboxylic acids) and the like.

Peptides can have mixed sequences or be composed of a single amino acid, e.g., poly(lysine). An exemplary polysaccharide is poly(sialic acid). An exemplary poly(ether) is poly(ethylene glycol), e.g. m-PEG. Poly(ethyleneimine) is an exemplary polyamine, and poly(acrylic) acid is a representative poly(carboxylic acid). The polymer backbone of the water-soluble polymer can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein. The polymer backbone can be linear or branched.

Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)ₘ in which R represents the core moiety, such as glycerol or pentaerythritol, and m represents the number of arms. Multiarmed PEG molecules, such as those described in U.S. Pat. No. 5,932,462, which is incorporated by reference herein in its entirety, can also be used as the polymer backbone.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as poly(propylene glycol) (“PPG”), copolymers of ethylene glycol and propylene glycol and the like, poly(oxyethylated polyol), poly(olefinic alcohol), polyvinylpyrrolidone), polylysine, polyethyleneimine, poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, and copolymers, terpolymers, and mixtures thereof. Although the molecular weight of each chain of the polymer backbone can vary, it is typically in the range of about 100 Da to about 100,000 Da.

In other embodiments, the scaffold moiety may be a peptide, serum albumin, thioredoxin, an immunoglobulin, an amino acid, a nucleic acid, a glycan, a modifying group that contains a reactive linker, a water-soluble polymer, a small carbon chain linker, or an additional therapeutic compound. In one embodiment, the scaffold moieties are non-toxic to humans and animals. In another embodiment, the scaffolds are endogenous serum proteins. In another embodiment, the scaffold moieties are water-soluble polymers. In another embodiment, the scaffolds are non-naturally-occurring polymers. In another embodiment, the scaffolds are naturally-occurring moieties that are modified by covalent attachment to additional moieties (e.g., PEG, poly(propylene glycol), poly(aspartate), biomolecules, therapeutic moieties, or diagnostic moieties).

The conjugation of hydrophilic polymers, such as PEG is known in the art. In its most common form, PEG is a linear polymer terminated at each end with hydroxyl groups: HO—CH₂CH₂O—(CH₂CH₂O)ₙ—CH₂CH₂—OH where n typically ranges from about 3 to about 4000. In a preferred embodiment, the PEG has a molecular weight distribution that is essentially homodisperse. In another preferred embodiment, the PEG is a linear polymer. In another preferred embodiment the PEG is a branched polymer.

Many end-functionalized or branched derivatives and various sizes are known in the art and commercially available. By way of example, conjugation of the PEG or PEO may be carried out using the compositions and methods described herein and in U.S. Pat. No. 7,803,777 (Defrees et al.) and U.S. Pat. No. 4,179,337 (Davis et al.), each of which are incorporated by reference herein in their entirety.

In some embodiments, smaller therapeutic compounds are paired with smaller scaffold moieties and larger therapeutic compounds are paired with larger scaffold moieties. It is contemplated, however, that smaller therapeutic compounds could be paired with a larger scaffold moiety and vice versa. Smaller therapeutic compounds are defined as having a molecular weight of 1 Da to 10 kDa. Larger therapeutic compounds are defined as having a molecular weight of 10 kDa to 1000 kDa.

The scaffolds of the present invention, for example, could have a molecular weight of 100 Daltons (Da.), 500 Da., 1000 Da., 2000 Da., 5000 Da., 10,000 Da., 15,000 Da., 20,000 Da., 30,000 Da., 40,000 Da. or 60,000 Da. In one embodiment of the invention, “small” scaffold moieties may be between about 100 Da. and 20,000 Da. In another embodiment, “large” scaffold moieties may be greater than about 20,000 Da. to about 200,000 Da. In preferred embodiments, the scaffold moiety is between about 100 Da. and 200,000 Da. In more preferred embodiments, the scaffold moiety is between about 100 Da. and 20,000 Da., 200 Da. and 15,000 Da., 300 Da. and 10,000 Da., 400 Da. and 9,000 Da., 500 Da. and 5,000 Da., 600 Da. and 2,000 Da., 1000 Da. and 200,000 Da., 20,000 Da. and 200,000 Da., 100,000 and 200,000 Da., 5000 Da. and 100,000 Da., 10,000 Da. and 80,000 Da., 20,000 Da. and 60,000 Da., or 20,000 Da. and 40,000 Da.

In other embodiments, the scaffold moiety may be a peptide, serum albumin, thioredoxin, an immunoglobulin, an amino acid, a nucleic acid, a glycan, a modifying group that contains a reactive linker, a water-soluble polymer, a small carbon chain linker, or an additional therapeutic compound. In one embodiment, the scaffold moieties are non-toxic to humans and animals. In another embodiment, the scaffolds are endogenous serum proteins. In another embodiment, the scaffold moieties are water-soluble polymers. In another embodiment, the scaffolds are non-naturally-occurring polymers. In another embodiment, the scaffolds are naturally-occurring moieties that are modified by covalent attachment to additional moieties (e.g., PEG, poly(propylene glycol), poly(aspartate), biomolecules, therapeutic moieties, or diagnostic moieties). The scaffolds and linkers of the invention are stable (i.e. non-releasable).

In certain embodiments, however, they may be “releasable” under specific condition. In preferred embodiments, the conjugation of the therapeutic compound retains substantially all of its activity following the conjugation. The active region of given therapeutic may be known in the art or determined empirically. In other embodiments, the conjugate is therapeutically active while remaining linked to the carrier. This embodiment may maximize the time in circulation and as well as its efficacy.

The scaffolds of the present invention, for example, could have a molecular weight of 100 Daltons (Da.), 500 Da., 1000 Da., 2000 Da., 5000 Da., 10,000 Da., 15,000 Da., 20,000 Da., 30,000 Da., 40,000 Da. or 60,000 Da. In one embodiment of the invention, “small” scaffolds may be between about 100 Da. and 20,000 Da. In another embodiment, “large” scaffolds may be greater than about 20,000 Da. to about 200,000 Da. In preferred embodiments, the scaffold moiety is between about 100 Da. and 200,000 Da. In more preferred embodiments, the scaffold is between about 100 Da. and 20,000 Da., 200 Da. and 15,000 Da., 300 Da. and 10,000 Da., 400 Da. and 9,000 Da., 500 Da. and 5,000 Da., 600 Da. and 2,000 Da., 1000 Da. and 200,000 Da., 20.00 Da. and 200,000 Da., 100,000 and 200,000 Da., 5000 Da. and 100,000 Da., 10,000 Da. and 80,000 Da., 20,000 Da. and 60,000 Da., or 20,000 Da. and 40,000 Da. The size of the scaffolds may be varied to maximize absorption, bioavailability, circulating half-life, or efficacy of the conjugated therapeutic compound.

Another component of the carrier molecule preferably comprises a coupling group that is used to covalently attach the drug to the scaffold or the carrier. The coupling groups of the invention include an amine-reactive group, a thiol-reactive group, a maleimide group, a thiol group, an aldehyde group, an NHS-ester group, a haloacetyl group, an iodoacetyl group, a bromoacetyl groups, a SMCC group, a sulfo SMCC group, a carbodiimide group and bifunctional cross-linkers such as NHS-Maleimido, combinations thereof, or other coupling groups familiar to persons skilled in the art. The coupling groups of the invention can promote thiol linkages, amide linkages, oxime linkages, hydrazone linkages, thiazolidinone linkages or utilizes cycloaddition reactions also called click chemistry to couple the carrier to a therapeutic compound. In another embodiment, the composition preferably includes a combination of one or more therapeutic compounds attached to the coupling group of the scaffold molecule.

The linkers of the invention may be between about 40 and 100 Daltons. In preferred embodiments, the linkers may be between about 40-50, 50-60, 60-70, 70-80, 80-90, or 90-100 Daltons. The linkers may also be varied to affect the stability or releasability of the link between the carrier and the therapeutic compound.

NHS groups are known to those skilled in the art as being useful for coupling to native peptides and proteins without having to engineer in a site of attachment. NHS groups allow attachment to most proteins and peptides that contain amino acids with amine groups such as a lysine residue. Utilization of NHS groups allows for flexibility in the site of carrier conjugation as protein structure and reaction time can influence the attachment site and number of carrier molecules conjugated to the therapeutic compound. By way of example, controlling the molar ratio of NHS-carrier to therapeutic compound, one skilled in the art can have some control over the number of carrier molecules attached to the therapeutic compound thus allowing for more than one carrier to be conjugated to a given therapeutic compound, if desired.

Conjugation of the carrier to a therapeutic compound is achieved by mixing a solution of the molecules together in a specific molar ratio using compatible solutions, buffers or solvents. For example, a molar ratio of 1:1, 2:1, 4:1, 5:1, 10:1, 20:1, 25:1, 50:1, 100:1, 1000:1, or 1:2, 1:4, 1:5, 1:10, 1:20 1:25, 1:50, 1:100 or 1:1000 of carrier to therapeutic compound could be used. In certain embodiments, a molar ratio of 1:1, 2:1, 4:1, 5:1, 10:1, 20:1, 25:1 or 1:2, 1:4, 1:5, 1:10, 1:20 1:25, 1:50 of carrier to therapeutic compound could be used. In preferred embodiments, a molar ratio of 1:1, 2:1, 4:1, 5:1, 10:1 or 1:2, 1:4, 1:5, 1:10 of carrier to therapeutic compound could be used. By varying the ratio, this could result in different numbers of individual carriers attached to the therapeutic compound, or could help to select a specific site of attachment. Attachment of the carriers is also pH, buffer, salt and temperature dependent and varying these parameters among other parameters can influence the site of attachment the number of carriers attached and the speed of the reaction. For example, by selecting a pH for the reaction at or below pH 6 could help selectively conjugate an aldehyde version of the carrier to the N-terminus of the therapeutic protein or peptide.

In certain embodiments, the present invention provides carriers that include those of formula I:

B-L¹-S-L²-L³-C I

Wherein:
B is a targeting group selected from vitamin D, a vitamin D analog, a vitamin D-related metabolite, an analog of a vitamin D related-metabolite, a peptide that binds DBP, an anti-DBP antibody, an anti-DBP antibody derivative, a nucleotide aptamer that binds DBP, or a small carbon-based molecule that binds DBP;
S is a scaffold moiety, comprising poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, polylactic acid, a water-soluble polymer, a small carbon chain linker, or an additional therapeutic compound;
C is an amine-reactive group, a thiol-reactive group, a maleimide group, a thiol group, a disulfide group, an aldehyde group, an NHS-ester group, a 4-nitrophenyl ester, an acylimidazole, a haloacetyl group, an iodoacetyl group, a bromoacetyl groups, a SMCC group, a sulfo SMCC group, a carbodiimide group and bifunctional cross-linkers such as NHS-Maleimido or combinations thereof,
L¹ and L² are linkers independently selected from —(CH₂)ₙ—, —C(O)NH—, —HNC(O)—, —C(O)O—, —OC(O)—, —O—, —S—S—, —S—, —S(O)—, —S(O)₂— and —NH—;
L³ is —(CH₂)ₒ—;
n is an integer from 0-3; and
is an integer from 0-3.

In preferred embodiments, the present invention provides carriers that include those of formula I:

B-L¹-S-L²-L³-C I

Wherein:
B is a targeting group selected from vitamin D, a vitamin D analog, a vitamin D-related metabolite, an analog of a vitamin D related-metabolite, or a small carbon-based molecule that binds DBP;
S is a scaffold moiety, comprising poly(ethylene glycol), polylysine, poly(propyleneglycol), a peptide, serum albumin, an amino acid, a nucleic acid, a glycan, polylactic acid, a water-soluble polymer, or a small carbon chain linker;
C is a maleimide group, a thiol group, a disulfide group, an aldehyde group, an NHS-ester group, an iodoacetyl group, or a bromoacetyl group;
L¹ and L² are linkers independently selected from —(CH₂)ₙ—, —C(O)NH—, —HNC(O)—, —C(O)O—, —OC(O)—, —O—, —S—, and —NH—;
L³ is —(CH₂)ₒ—;
n is an integer from 0-3; and
is an integer from 0-3.

In more preferred embodiments, the present invention provides carriers that include those of formula I:

B-L¹-S-L²-L³-C I

Wherein:
B is a targeting group selected from vitamin D, a vitamin D analog, or a vitamin D-related metabolite;
S is a scaffold moiety, comprising poly(ethylene glycol), polylysine or poly(propyleneglycol);
C is a maleimide group, a disulfide group, an aldehyde group, an NHS-ester group or an iodoacetyl group;
L¹ and L² are linkers independently selected from —(CH₂)ₙ—, —C(O)NH—, —HNC(O)—, —C(O)O— and —OC(O)—;
L³ is —(CH₂)ₒ—;
n is an integer from 0-3; and
is an integer from 0-3.

In most preferred embodiments, the present invention provides carriers that include those of formulas IIa and IIb:

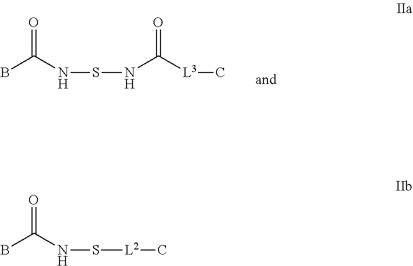

Wherein:
B is a targeting group selected from vitamin D, a vitamin D analog, or a vitamin D-related metabolite;
S is a scaffold moiety, comprising poly(ethylene glycol), or poly(propyleneglycol); and
C is a maleimide group, a disulfide group, an aldehyde group, an NHS-ester group or an iodoacetyl group;
L² is —(CH₂)ₙ—;
L³ is —(CH₂)ₒ—;
n is 1; and
o is 2.

In certain most preferred embodiments of formula IIa, B is represented by formula III, S is poly(ethylene glycol) and L³-C is represented by formula IVa.

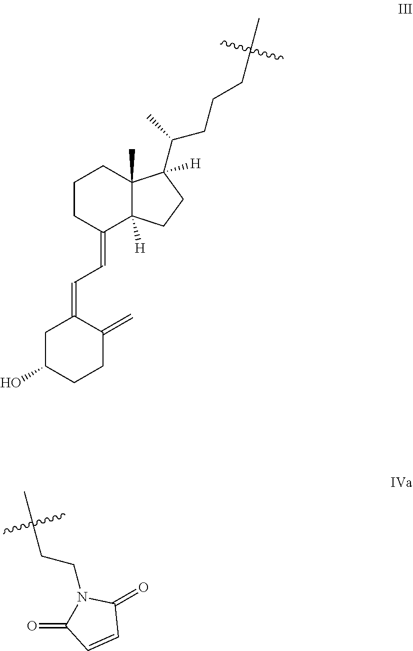 [SMILES: *CCC[C@@H](C)[C@H]1CC[C@H]2/C(=C/C=C3/C[C@@H](O)CCC3=C)CCC[C@]12C ; *CCN1C(=O)C=CC1=O]

In certain most preferred embodiments of formula IIb, B is represented by formula III, S is poly(ethylene glycol) and L²-C is represented by formula IVb.

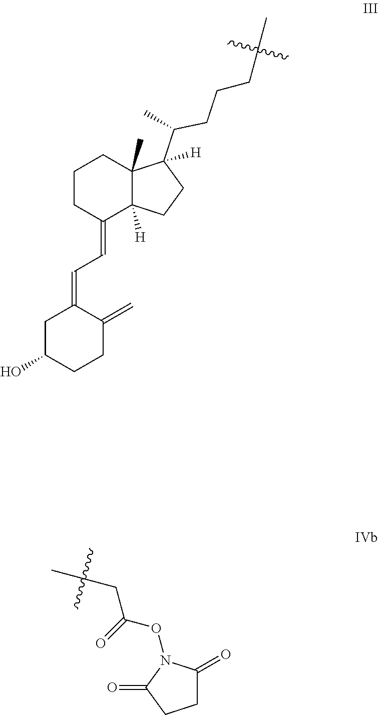 [SMILES: *CCC[C@@H](C)[C@H]1CC[C@H]2/C(=C/C=C3/C[C@@H](O)CCC3=C)CCC[C@]12C ; *CC(=O)ON1C(=O)CCC1=O]

In certain most preferred embodiment, S is between about 100 Da. and 200,000 Da. In other most preferred embodiments, the scaffold moiety is between about 100 Da. and 20,000 Da., 200 Da. and 15,000 Da., 300 Da. and 10,000 Da., 400 Da. and 9,000 Da., 500 Da. and 5,000 Da., 600 Da. and 2,000 Da., 1000 Da. and 200,000 Da., 5000 Da. and 100,000 Da., 10,000 Da. and 80,000 Da., 20,000 Da. and 60,000 Da., or 20,000 Da. and 40,000 Da.

In a specific embodiment, the present invention provides a carrier represented by formula V.

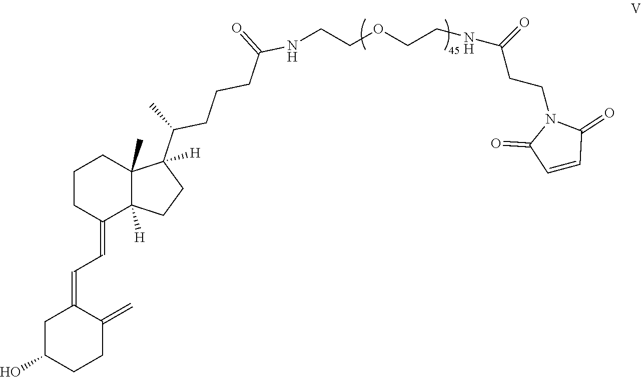

In another specific embodiment, the present invention provides a carrier represented by formula VI.

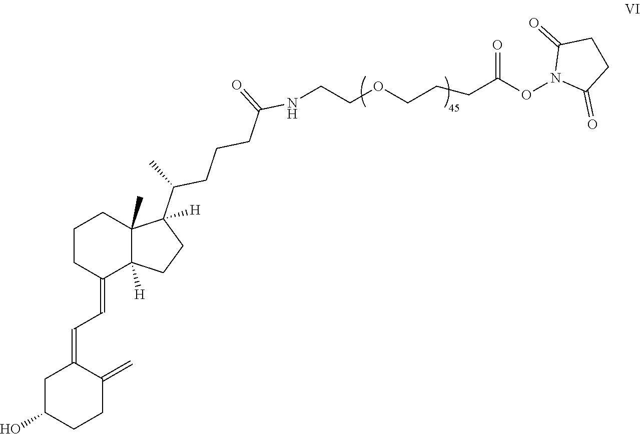

In certain embodiments, the present invention provides a method for producing a carrier of formula I:

B-L¹-S-L²-L³-C I

comprising the step of reacting a compound of formula Ia:

B—COOH Ia

with a compound of formula Ib:

H₂N—S-L²-L³-C Ib

in the presence of an amide coupling agent,

wherein B, S, C and L² are defined as above and L¹ is —C(O)NH—.

One skilled in the art will recognize that a compound of formula Ib can be used either as a free base or as a suitable salt form. Suitable salt forms include, but are not limited to TFA, HCl, HBr, MsOH, TfOH and AcOH.

Any suitable amide coupling agent may be used to form a compound of formula I. Suitable amide coupling agents include, but are not limited to 2-chloromethylpyridinium iodide, BOP, PyBOP, HBTU, HATU, DCC, EDCI, TBTU and T3P.

In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine.

In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

In certain embodiments, the carboxylic acid component of formula Ia is produced by treating an ester of formula Id with a hydrolyzing agent:

B—COOR Id

wherein, B is defined as above and R is a C₁-C₆ branched or unbranched alkyl group.

Any suitable hydrolyzing agent can be used to prepare a compound of formula Ia from a compound of formula Id.

In certain other embodiments, the present invention provides a method for producing a carrier of formula Ig:

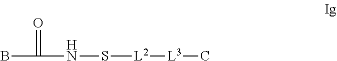

comprising the steps of reacting a compound of formula Ia:

B—COOH Ia

with a compound of formula Ic:

H₂N—S-L²-L³-COOR¹ Ic

in the presence of an amide coupling agent forming a compound of formula Ie; Hydrolyzing an ester of formula Ie to a carboxylic acid of formula If, and

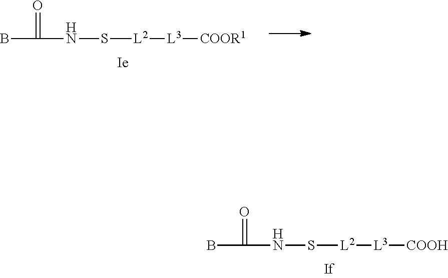

Converting a carboxylic acid of formula If to an active ester of formula I;

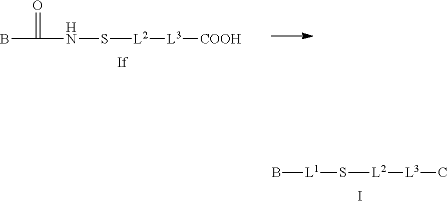

wherein B, S, C, R¹, L², L³, n and o are defined as above and L¹ is —C(O)NH—.

One skilled in the art will recognize that a compound of formula Ic can be used either as a free base or as a suitable salt form. Suitable salt forms include, but are not limited to TFA, HCl, HBr, MsOH, TfOH and AcOH.

Any suitable amide coupling agent may be used to form a compound of formula Ie. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone.

In certain embodiments, the amide coupling agent is used with a co-reagent such as HOBT or DMAP.

In certain embodiments, the amide coupling agent is used with a base such as triethylamine or diisopropylethylamine.

In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

In certain embodiments, the carboxylic acid component of formula Ia is produced by treating an ester of formula Id with a hydrolyzing agent:

B—COOR Id

wherein, B is defined as above and R is a C₁-C₆ branched or unbranched alkyl group.

Any suitable hydrolyzing agent can be used to prepare a compound of formula Ia from a compound of formula Id. Suitable hydrolyzing agents include, but are not limited to lithium hydroxide, sodium hydroxide and potassium hydroxide.

Any suitable hydrolyzing agent can be used to prepare a compound of formula If from a compound of formula Ie. Suitable hydrolyzing agents include, but are not limited to lithium hydroxide, sodium hydroxide and potassium hydroxide.

Any suitable leaving group can be coupled with a carboxylic acid of formula If in the presence of a suitable coupling reagent to form an active ester of formula I. Suitable leaving groups include, but are not limited to imidazole, HOBT, NHS and 4-nitrophenol. Suitable coupling reagents include, but are not limited to 2-chloromethylpyridinium iodide, BOP, PyBOP, HBTU, HATU, DCC, EDCI, TBTU and T3P.

In some embodiments, an active ester of formula I is formed from a carboxylic acid of formula If using a combination of a suitable leaving group and a coupling reagent.

In some embodiments, an active ester of formula I is formed from a carboxylic acid of formula If using a single reagent that produces a leaving group and also effects a coupling reaction. Such reagents include, but are not limited to 1,1′-carbonyldiimidazole, N,N′-disuccinimidyl carbonate, 4-nitrophenyl trifluoroacetate and HBTU. In some embodiments, the single reagent is used alone. In other embodiments, the single reagent is used with an acyl transfer catalyst. Such acyl transfer catalysts include, but are not limited to DMAP and pyridine. One skilled in the art will recognize that additional acyl transfer catalysts may be used.

In a specific embodiment, the present invention provides a method for producing a carrier represented by formula V:

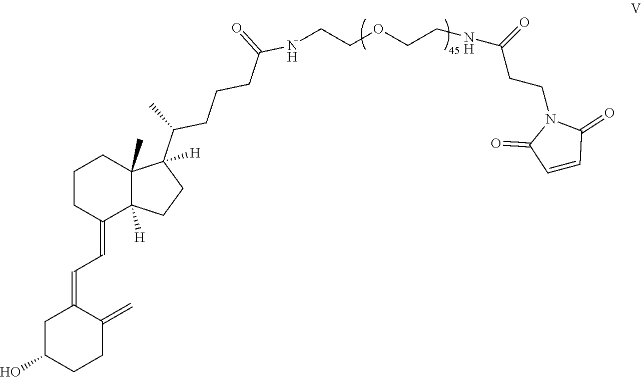

comprising the step of reacting a compound of formula Va:

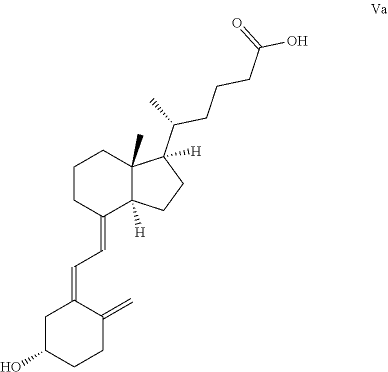 [SMILES: C=C1CC[C@H](O)C/C1=C/C=C1\CCC[C@]2(C)[C@@H]([C@H](C)CCCC(=O)O)CC[C@@H]12]

with a compound of formula Vb:

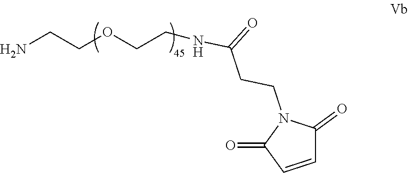

in the presence of an amide coupling agent. One skilled in the art will recognize that a compound of formula Vb can be used either as a free base or as a suitable salt form. Suitable salt forms include, but are not limited to TFA, HCl, HBr, MsOH, TfOH and AcOH.

Any suitable amide coupling agent may be used to form a compound of formula V. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine. In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

In a specific embodiment, the carboxylic acid component of formula Va is produced by treating a methyl ester of formula Vc with a hydrolyzing agent:

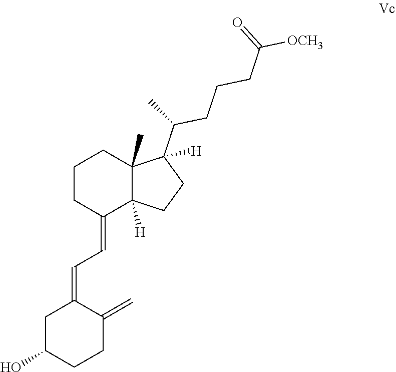 [SMILES: C=C1CC[C@H](O)C/C1=C/C=C1\CCC[C@]2(C)[C@@H]([C@H](C)CCCC(=O)OC)CC[C@@H]12]

Any suitable hydrolyzing agent can be used to prepare a compound of formula Va from a compound of formula Vc. Suitable hydrolyzing agents include, but are not limited to lithium hydroxide, sodium hydroxide and potassium hydroxide.

In another specific embodiment, the present invention provides a method for producing a carrier represented by for producing a carrier represented by formula VI:

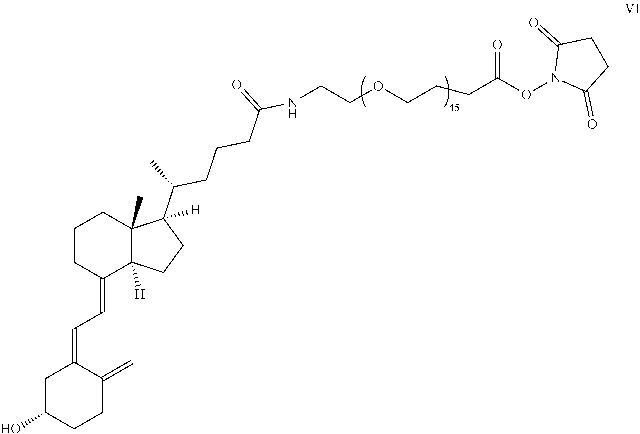

comprising the steps of reacting a compound of formula Va:

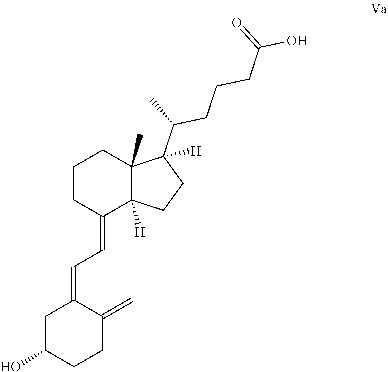 [SMILES: C=C1CC[C@H](O)C/C1=C/C=C1\CCC[C@]2(C)[C@@H]([C@H](C)CCCC(=O)O)CC[C@@H]12]

with a compound of formula VIa:

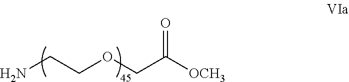

in the presence of an amide coupling agent forming a compound of formula VIb; Hydrolyzing an ester of formula VIb to a carboxylic acid of formula VIc; and

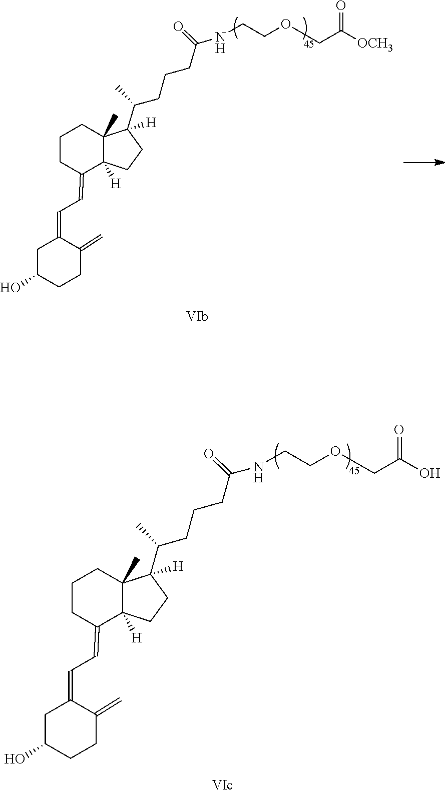

Converting a carboxylic acid of formula VIe to an active ester of formula VI;

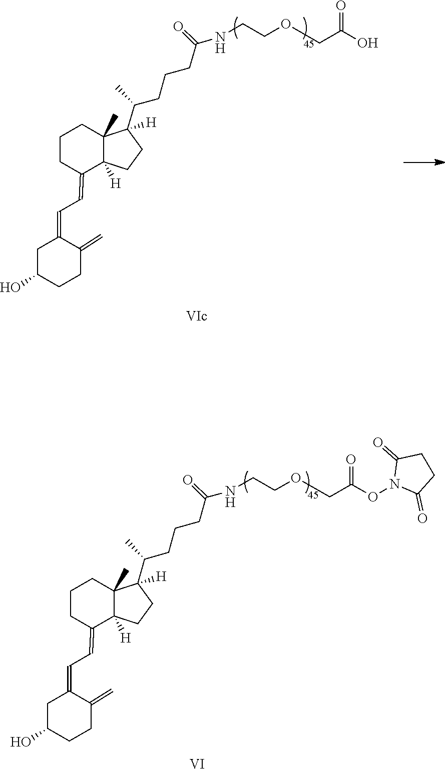

One skilled in the art will recognize that a compound of formula VIa can be used either as a free base or as a suitable salt form. Suitable salt forms include, but are not limited to TFA, HCl, HBr, MsOH, TfOH and AcOH.

Any suitable amide coupling agent may be used to form a compound of formula VIb. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine. In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

Any suitable hydrolyzing agent can be used to prepare a compound of formula VIc from a compound of formula VIb. Suitable hydrolyzing agents include, but are not limited to lithium hydroxide, sodium hydroxide and potassium hydroxide.

NHS can be coupled with a carboxylic acid of formula VIc in the presence of a suitable coupling reagent to form an active ester of formula VI. Suitable coupling reagents include, but are not limited to 2-chloromethylpyridinium iodide, BOP, PyBOP, HBTU, HATU, DCC, EDCI, TBTU and T3P.

In some embodiments, an active ester of formula VI is formed from a carboxylic acid of formula VIc using a combination of NHS and a coupling reagent.

In some embodiments, an active ester of formula VI is formed from a carboxylic acid of formula VIc using a single reagent that produces a leaving group and also effects a coupling reaction. Such reagents include, but are not limited to, N,N′-disuccinimidyl carbonate. In some embodiments, the single reagent is used alone. In other embodiments, the single reagent is used with an acyl transfer catalyst. Such acyl transfer catalysts include, but are not limited to DMAP and pyridine. One skilled in the art will recognize that additional acyl transfer catalysts may be used.

In a specific embodiment, the carboxylic acid component of formula Va is produced by treating a methyl ester of formula Vc with a hydrolyzing agent:

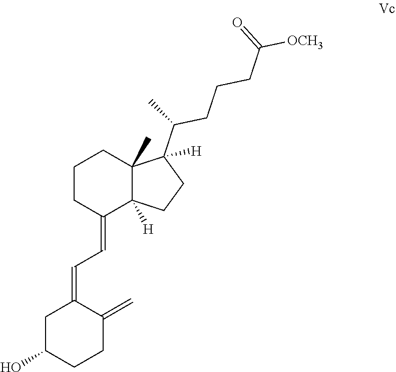 [SMILES: C=C1CC[C@H](O)C/C1=C/C=C1\CCC[C@]2(C)[C@@H]([C@H](C)CCCC(=O)OC)CC[C@@H]12]

Any suitable hydrolyzing agent can be used to prepare a compound of formula Va from a compound of formula Vc. Suitable hydrolyzing agents include, but are not limited to lithium hydroxide, sodium hydroxide and potassium hydroxide.

In certain embodiments, the present invention provides carriers that include those of formula I:

B-(L)^{a}-S-(M)^{b}-C

Wherein:

B is a targeting group selected from vitamin D, a vitamin D analog, a vitamin D-related metabolite, an analog of a vitamin D related-metabolite, a peptide that binds DBP, an anti-DBP antibody, an anti-DBP antibody derivative, a nucleotide aptamer that binds DBP, or a small carbon-based molecule that binds DBP; S is a scaffold moiety, comprising poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, polylactic acid, a water-soluble polymer, a small carbon chain linker, or an additional therapeutic compound; C is an amine-reactive group, a thiol-reactive group, a maleimide group, a thiol group, a disulfide group, an aldehyde group, an NHS-ester group, a 4-nitrophenyl ester, an acylimidazole, a haloacetyl group, an iodoacetyl group, a bromoacetyl groups, a SMCC group, a sulfo SMCC group, a carbodiimide group and bifunctional cross-linkers such as NHS-maleimido or combinations thereof;

(L)^{a} and (M)^{b} are linkers independently selected from —(CH₂)ₙ—, —C(O)NH—, —HNC(O)—, —C(O)O—, —OC(O)—, —O—, —S—S—, —S—, —S(O), —S(O)₂— and —NH—;

a is an integer from 0-4; and

b is an integer from 0-4; and

n is an integer from 0-3.

In preferred embodiments, the present invention provides carriers that include those of formula I:

B-(L)^{a}-S-(M)^{b}-C I

Wherein:

B is a targeting group selected from vitamin D, a vitamin D analog, a vitamin D-related metabolite, an analog of a vitamin D related-metabolite, or a small carbon-based molecule that binds DBP;

S is a scaffold moiety, comprising poly(ethylene glycol), polylysine, poly(propyleneglycol), a peptide, serum albumin, an amino acid, a nucleic acid, a glycan, polylactic acid, a water-soluble polymer, or a small carbon chain linker;

C is a maleimide group, a thiol group, a disulfide group, an aldehyde group, an NETS-ester group, an iodoacetyl group, or a bromoacetyl group; (L)^{a} and (M)^{b} are linkers independently selected from —(CH₂)ₙ—, —C(O)NH—, —HNC(O)—, —C(O)O—, —OC(O)—, —O—, —S—S—, —S—, —S(O)—, —S(O)₂— and —NH—;

a is an integer from 0-4; and

b is an integer from 0-4; and

n is an integer from 0-3.

In more preferred embodiments, the present invention provides carriers that include those of formula I:

B-(L)^{a}-S-(M)^{b}-C I

Wherein:

B is a targeting group selected from vitamin D, a vitamin D analog, or a vitamin D-related metabolite;

S is a scaffold moiety, comprising poly(ethylene glycol), polylysine or poly(propyleneglycol);

C is a maleimide group, a disulfide group, an aldehyde group, an NHS-ester group or an iodoacetyl group;

(L)^{a} and (M)^{b} are linkers independently selected from —(CH₂)ₙ—, —C(O)NH—, —HNC(O)—, —C(O)O—, —OC(O)—, —O—, —S—S—, —S—, —S(O)—, —S(O)₂— and —NH—;

a is an integer from 0-4; and

b is an integer from 0-4; and

n is an integer from 0-3.

In most preferred embodiments, the present invention provides carriers that include those of formulas IIa, IIb, and IIc:

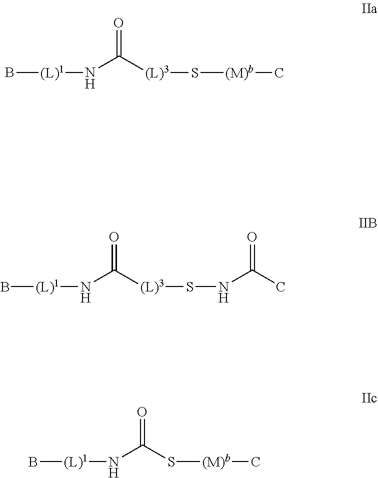

Wherein:

B is a targeting group selected from vitamin D, a vitamin D analog, or a vitamin D-related metabolite;

S is a scaffold moiety, comprising poly(ethylene glycol), or poly(propyleneglycol); and

C is a maleimide group, a disulfide group, an aldehyde group, an NHS-ester group or an iodoacetyl group;

L¹ is —(CH₂)ₙ—;

L³ is —(CH₂)ₒ—;

(M)^{b} are linkers independently selected from —(CH₂)ₙ—, —C(O)NH—, —HNC(O)—, —C(O)O—, —OC(O)—, —O—, —S—S—, —S—, —S(O)—, —S(O)₂— and —NH—;

b is an integer from 0-4; and

n is 3; and

o is 1.

In U.S. Pat. No. 9,289,507, which is incorporated herein by reference, conjugation at the Carbon 25 (C25) position of 25-hydroxy-vitamin D₃ is exemplified. The present invention incorporates conjugation at the C3 position of 25-hydroxy-vitamin D₃ as is exemplified in U.S. Pat. No. 9,595,934, which is incorporated herein by reference. This gives improved half-life extension and bioavailability compared to the C25 conjugates.

In certain most preferred embodiments of formula IIa, B is represented by formula III, S is poly(ethylene glycol) and (M)^{b}-C is represented by formula IVa.

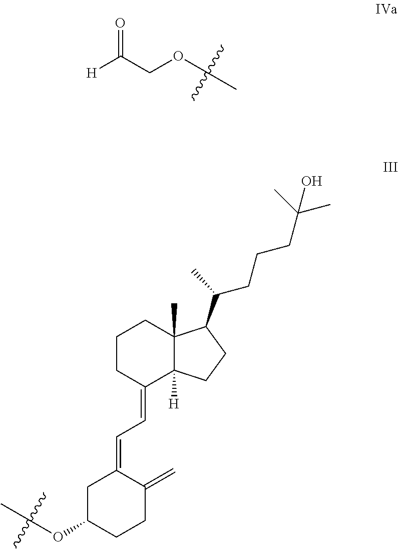 [SMILES: *OCC=O ; *O[C@H]1CCC(=C)/C(=C\C=C2/CCC[C@]3(C)[C@@H]([C@H](C)CCCC(C)(C)O)CC[C@@H]23)C1]

In certain most preferred embodiments of formula IIb, B is represented by formula III, S is poly(ethylene glycol) and (M)^{b}-C is represented by formula IVb.

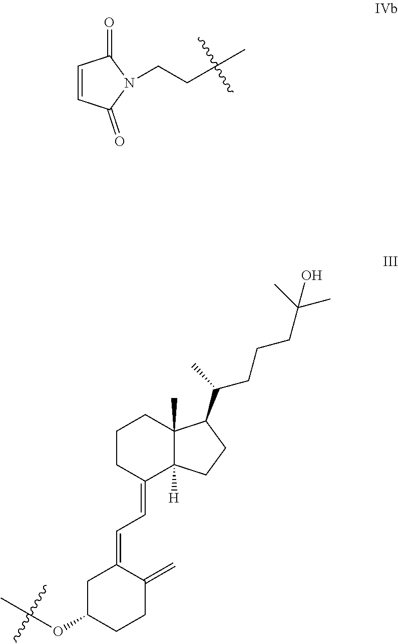 [SMILES: *CCN1C(=O)C=CC1=O ; *O[C@H]1CCC(=C)/C(=C\C=C2/CCC[C@]3(C)[C@@H]([C@H](C)CCCC(C)(C)O)CC[C@@H]23)C1]

In certain most preferred embodiments of formula IIc, B is represented by formula III, S is poly(ethylene glycol) and (M)^{b}-C is represented by formula IVc.

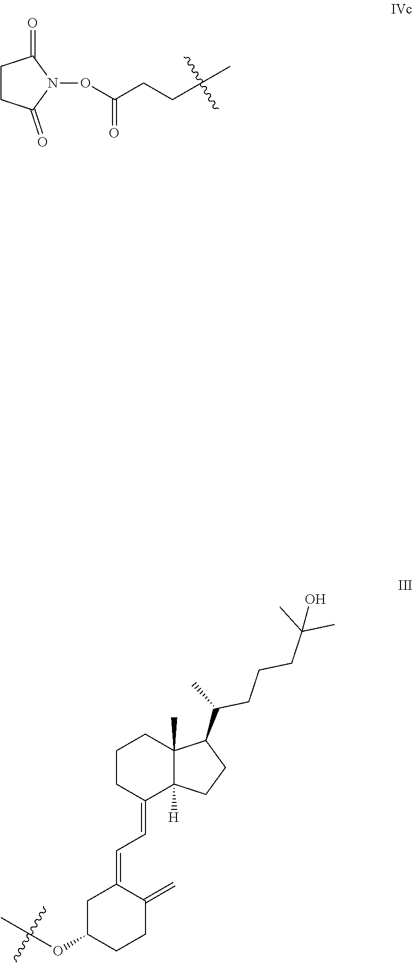 [SMILES: *CCC(=O)ON1C(=O)CCC1=O ; *O[C@H]1CCC(=C)/C(=C\C=C2/CCC[C@]3(C)[C@@H]([C@H](C)CCCC(C)(C)O)CC[C@@H]23)C1]

In certain most preferred embodiment, S is between about 100 Da. and 200,000 Da. In other most preferred embodiments, the scaffold moiety is between about 100 Da. and 20,000 Da., 200 Da. and 15,000 Da., 300 Da. and 10,000 Da., 400 Da. and 9,000 Da., 500 Da. and 5,000 Da., 600 Da. and 2,000 Da., 1000 Da. and 200,000 Da., 5000 Da. and 100,000 Da., 10,000 Da. and 80,000 Da., 20,000 Da. and 60,000 Da., or 20,000 Da. and 40,000 Da.

In a specific embodiment, the present invention provides a carrier represented by formula V.

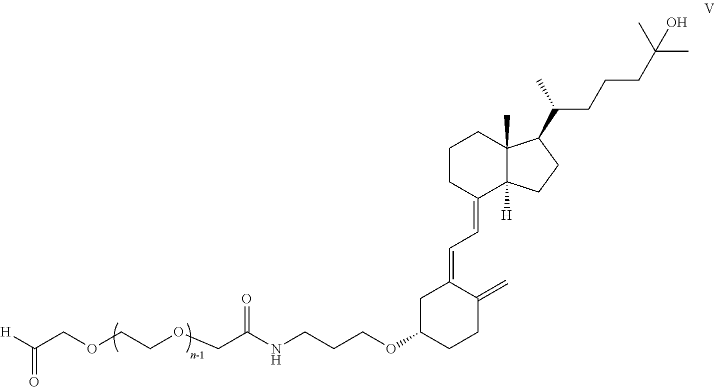

In another specific embodiment, the present invention provides a carrier represented by formula VI.

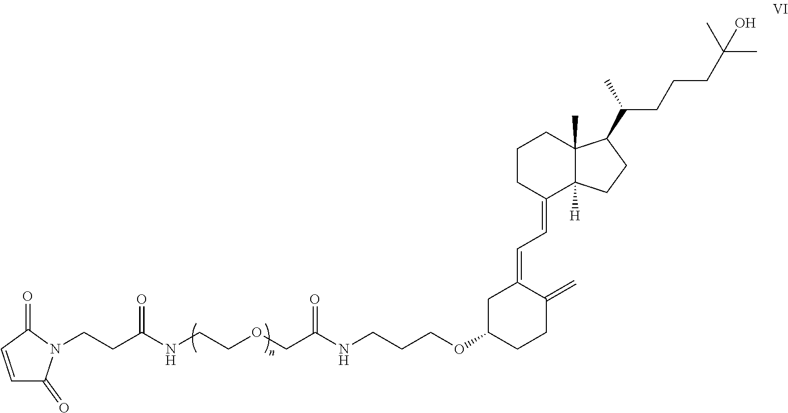

In another specific embodiment, the present invention provides a carrier represented by formula VII.

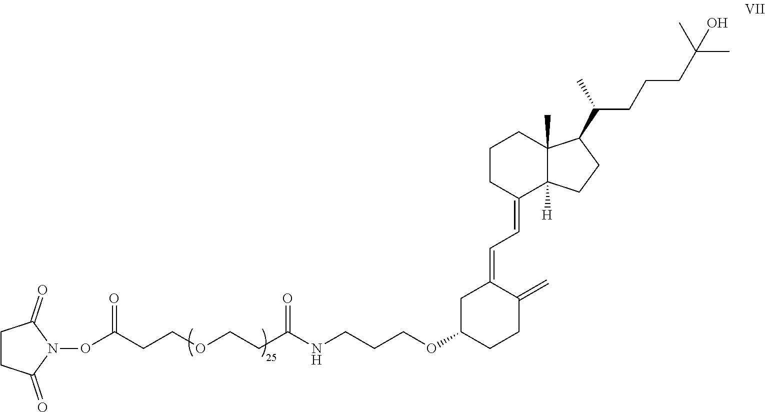

In certain embodiments, the present invention provides a method for producing a carrier of formula I:

B-(L)^{a}-S-(M)^{b}-C I

comprising the step of reacting a compound of formula Ia:

B-L¹-NH₂ Ia

with a compound of formula Ib:

HOOC-L³-S-(M)^{b}-C Ib

in the presence of an amide coupling agent,

wherein B, S, C and L¹, L³, and (M)^{b} are defined as above and L² is —C(O)NH—.

One skilled in the art will recognize that a compound of formula Ia can be used either as a free base or as a suitable salt form. Suitable salt forms include, but are not limited to TFA, HCl, HBr, MsOH, TfOH and AcOH.

Any suitable amide coupling agent may be used to form a compound of formula I. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine. In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

One skilled in the art will recognize that any suitable leaving group may be coupled with the carboxylic acid of formula Ib in the presence of a suitable coupling agent to form an active ester of formula Ic:

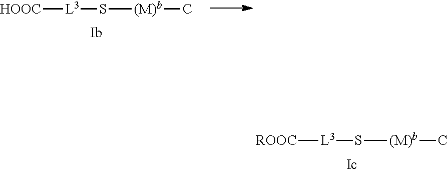

wherein R is a suitable leaving group including, but are not limited to imidazole, HOBT, NHS and 4-nitrophenol. Suitable coupling reagents include, but are not limited to 2-chloromethylpyridinium iodide, BOP, PyBOP, HBTU, HATU, DCC, EDCI, TBTU and T3P. In some embodiments, the present invention provides a method for producing a carrier of formula I:

B-(L)^{a}-S-(M)^{b}-C I

comprising the step of reacting a compound of formula Ia:

B—L¹-NH₂ Ia

with a compound of formula

ROOC-L³-S-(M)^{b}-C Ic

wherein B, S, C, R and L¹, L³, and (M)^{b} are defined as above and L² is —C(O)NH—.

One skilled in the art will recognize that a compound of formula Ia can be used either as a free base or as a suitable salt form. Suitable salt forms include, but are not limited to TFA, HCl, HBr, MsOH, TfOH and AcOH.

In certain embodiments, the amide coupling is performed with a base such as triethylamine or diisopropylethylamine. One skilled in the art will recognize that bases other than triethylamine or diisopropylethylamine may be used.

In certain other embodiments, the present invention provides a method for producing a carrier of formula IIa:

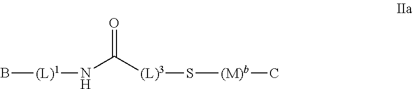

comprising the steps of reacting a compound of formula Ia:

B-L¹-NH₂ Ia

with a compound of formula Id:

HOOC-L³-S-(M)^{b}-CH₂OH Id

in the presence of an amide coupling agent forming a compound of formula Ie; and

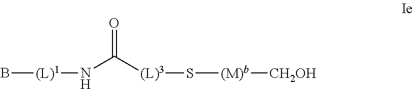

Oxidation of the primary alcohol of formula Ie to an aldehyde of formula IIa;

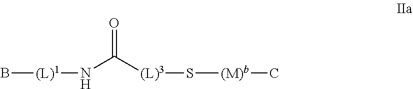

wherein B, S, L¹, L³, (M)^{b}, b, n and o are defined as above and L² is C(O)NH— and C is an aldehyde group.

Any suitable oxidizing agent may be used to form a compound of formula IIa. Suitable oxidizing agents include, but are not limited to, the Collins reagent, PDC, PCC, oxalyl chloride/DMSO (Swern oxidation), SO₃-pyridine/DMSO (Parikh-Doehring oxidation), Dess-Martin periodinane, TPAP/NMO, and TEMPO/NaOCl.

One skilled in the art will recognize that a compound of formula Ia can be used either as a free base or as a suitable salt form. Suitable salt forms include, but are not limited to TFA, HCl, HBr, MsOH, TfOH and AcOH.

Any suitable amide coupling agent may be used to form a compound of formula Ie. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine. In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

In certain embodiments, any suitable leaving group can be coupled with a carboxylic acid of formula Id in the presence of a suitable coupling reagent to form an active ester of formula If:

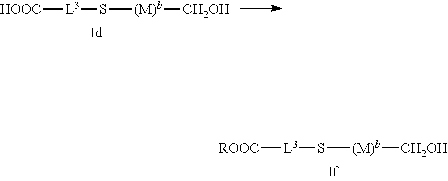

wherein R is a suitable leaving group including, but are not limited to imidazole, HOBT, NHS and 4-nitrophenol. Suitable coupling reagents include, but are not limited to 2-chloromethylpyridinium iodide, BOP, PyBOP, HBTU, HATU, DCC, EDCI, TBTU and T3P.

In some embodiments, the present invention provides a method for producing a carrier of formula Ie:

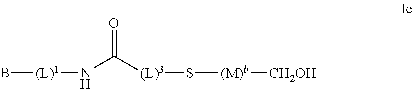

comprising the step of reacting a compound of formula Ia;

B-L¹-NH₂ Ia

with a compound of formula If, and

ROOC-L³-S-(M)^{b}-CH₂OH If

Oxidation of the primary alcohol of formula Ie to an aldehyde of formula IIa;

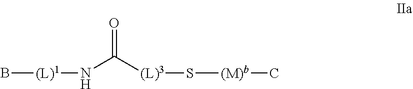

wherein B, S, C, R and L¹, L³, and (M)b are defined as above and L² is —C(O)NH—.

One skilled in the art will recognize that a compound of formula Ia can be used either as a free base or as a suitable salt form. Suitable salt forms include, but are not limited to TFA, HCl, HBr, MsOH, TfOH and AcOH.

In certain embodiments, the amide coupling is performed with a base such as triethylamine or diisopropylethylamine. One skilled in the art will recognize that bases other than triethylamine or diisopropylethylamine may be used.

Any suitable oxidizing agent may be used to form a compound of formula IIa. Suitable oxidizing agents include, but are not limited to, the Collins reagent, PDC, PCC, oxalyl chloride/DMSO (Swern oxidation), SO₃-pyridine/DMSO (Parikh-Doehring oxidation), Dess-Martin periodinane, TPAP/NMO, and TEMPO/NaOCl.

In certain other embodiments, the present invention provides a method for producing a carrier of formula IIc:

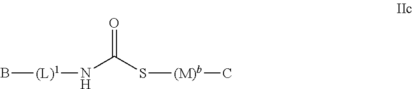

comprising the steps of reacting a compound of formula Ia:

B-L¹-NH₂ Ia

with a compound of formula Ig:

ROOC—S-(M)^{b}-COOH Ig

forming a compound of formula Ih; and

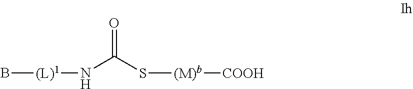

Converting a carboxylic acid of formula Ih to an active ester of formula IIc;

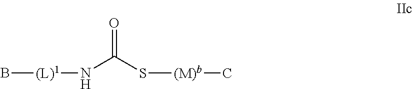

wherein B, S, C, R, L¹, (M)^{b}, b, n and o are defined as above and L² is —C(O)NH—.

One skilled in the art will recognize that a compound of formula Ia can be used either as a free base or as a suitable salt form. Suitable salt forms include, but are not limited to TFA, HCl, HBr, MsOH, TfOH and AcOH.

Any suitable leaving group can be coupled with a carboxylic acid of formula Ih in the presence of a suitable coupling reagent to form an active ester of formula IIc. Suitable leaving groups include, but are not limited to imidazole, HOBT, NHS and 4-nitrophenol. Suitable coupling reagents include, but are not limited to 2-chloromethylpyridinium iodide, BOP, PyBOP, HBTU, HATU, DCC, EDCI, TBTU and T3P. In some embodiments, an active ester of formula IIc is formed from a carboxylic acid of formula Ih using a combination of a suitable leaving group and a coupling reagent.

In some embodiments, an active ester of formula IIc is formed from a carboxylic acid of formula Ih using a single reagent that produces a leaving group and also effects a coupling reaction. Such reagents include, but are not limited to 1,1′-carbonyldiimidazole, N,N′-disuccinimidyl carbonate, 4-nitrophenyl trifluoroacetate and HBTU. In some embodiments, the single reagent is used alone. In other embodiments, the single reagent is used with an acyl transfer catalyst. Such acyl transfer catalysts include, but are not limited to DMAP and pyridine. One skilled in the art will recognize that additional acyl transfer catalysts may be used.

In a specific embodiment, the present invention provides a method for producing a carrier represented by formula V:

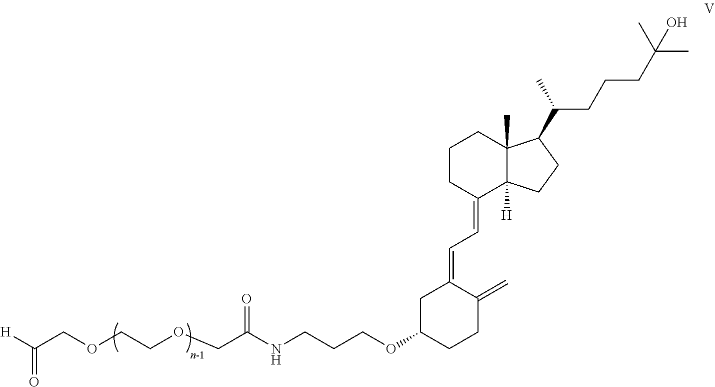

comprising the step of reacting a compound of formula Va:

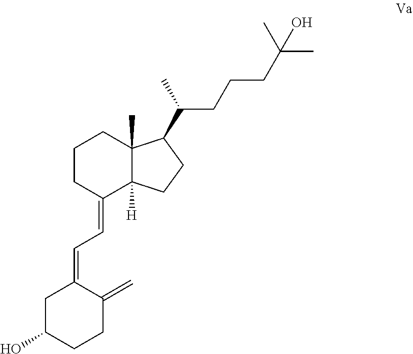 [SMILES: C=C1CC[C@H](O)C/C1=C/C=C1\CCC[C@]2(C)[C@@H]([C@H](C)CCCC(C)(C)O)CC[C@@H]12]

with a compound of formula Vb:

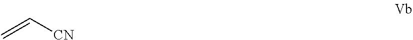

to form a compound of formula Vc;

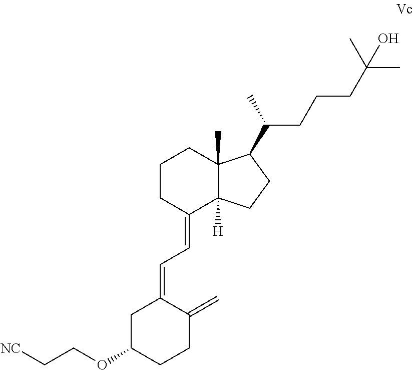 [SMILES: C=C1CC[C@H](OCCC#N)C/C1=C/C=C1\CCC[C@]2(C)[C@@H]([C@H](C)CCCC(C)(C)O)CC[C@@H]12]

Reduction of the nitrile group to form the amine of formula Vd.

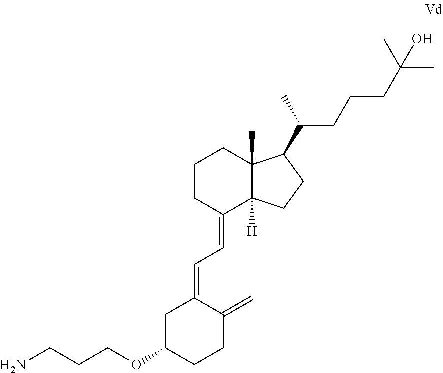 [SMILES: C=C1CC[C@H](OCCCN)C/C1=C/C=C1\CCC[C@]2(C)[C@@H]([C@H](C)CCCC(C)(C)O)CC[C@@H]12]

Reaction of the compound of formula Vd with a compound of formula Ve;

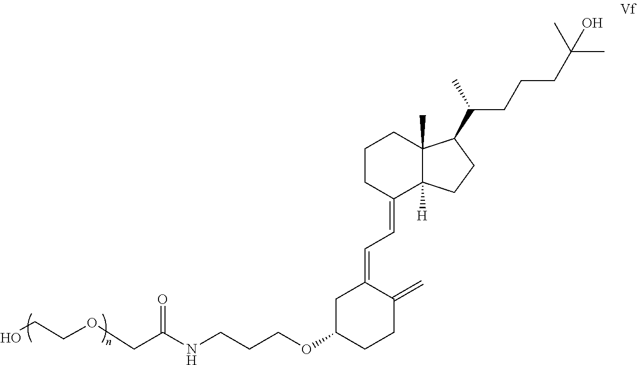

To form a compound of the formula Vf

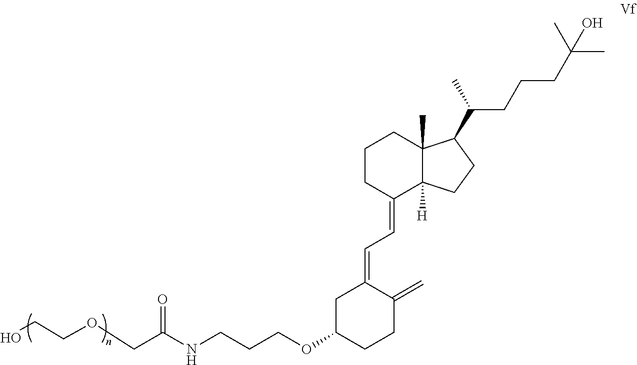

Oxidation of the primary alcohol of formula Vf to form the aldehyde of formula V.

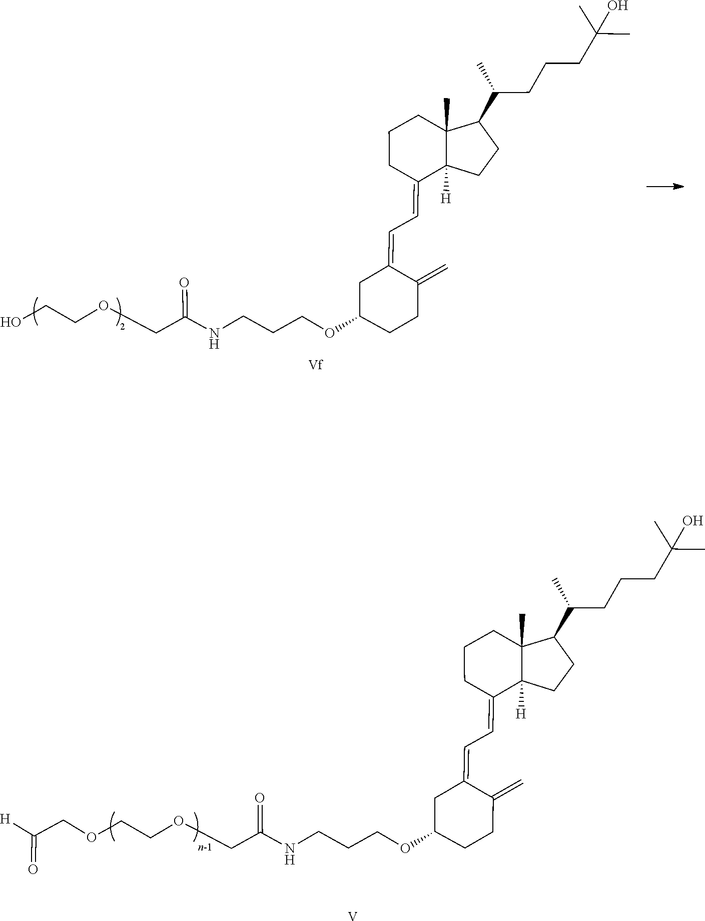

In some embodiments, the reaction of a compound of formula Vb with a compound of formula Va is promoted by addition of Triton B. One skilled in the art will recognize that other reagents may be used to promote nucleophilic addition to acrylonitrile. In some embodiments, reduction of the nitrile of formula Vc to the amine of formula Vd is performed using AlCl₃/LAH. One skilled in the art will recognize that other reduction reagents may be used including sodium, H₂/Pd, Hz/Raney nickel, and diborane.

One skilled in the art will recognize that a compound of formula Vd can be used either as a free base or as a suitable salt form. Suitable salt forms include, but are not limited to TFA, HCl, HBr, MsOH, TfOH and AcOH.

In certain embodiments, a base such as triethylamine or diisopropylethylamine is used to promote coupling of the NETS-ester of formula Ve with the amine of formula Vd. One skilled in the art will recognize that bases other than triethylamine or diisopropylethylamine may be used. Any suitable oxidizing agent may be used to form a compound of formula V. Suitable oxidizing agents include, but are not limited to, the Collins reagent, PDC, PCC, oxalyl chloride/DMSO (Swern oxidation), SO₃-pyridine/DMSO (Parikh-Doehring oxidation), Dess-Martin periodinane, TPAP/NMO, and TEMPO/NaOCl.

In another specific embodiment, the present invention provides a method for producing a carrier represented by formula VI:

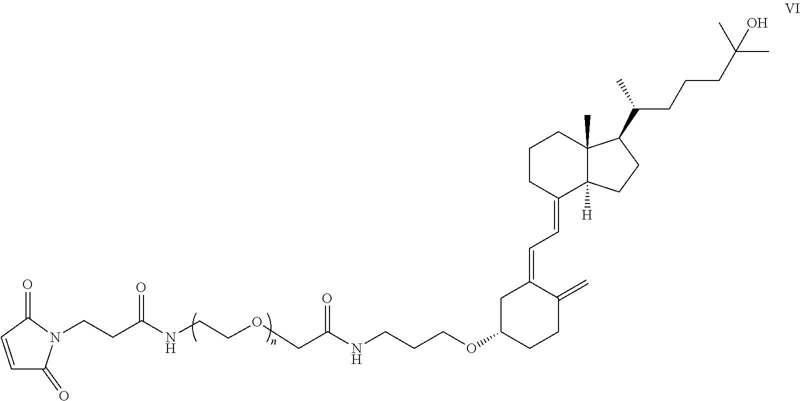

comprising the steps of reacting a compound of formula Vd:

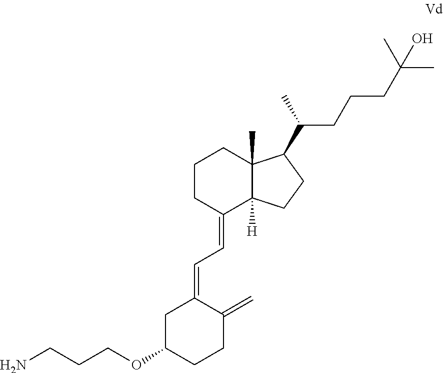 [SMILES: C=C1CC[C@H](OCCCN)C/C1=C/C=C1\CCC[C@]2(C)[C@@H]([C@H](C)CCCC(C)(C)O)CC[C@@H]12]

in the presence of an amide coupling agent with a compound of formula VIa:

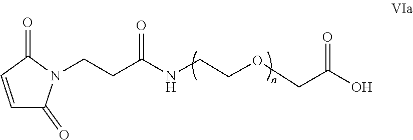

One skilled in the art will recognize that a compound of formula Vd can be used either as a free base or as a suitable salt form. Suitable salt forms include, but are not limited to TFA, HCl, HBr, MsOH, TfOH and AcOH.

Any suitable amide coupling agent may be used to form a compound of formula VI. Suitable amide coupling agents include, but are not limited to 2-chloromethylpyridinium iodide, BOP, PyBOP, HBTU, HATU, DCC, EDCI, TBTU and T3P. In certain embodiments, the amide coupling agent is used alone. In certain embodiments, the amide coupling agent is used with a co-reagent such as HOBT or DMAP. In certain embodiments, the amide coupling agent is used with a base such as triethylamine or diisopropylethylamine. In certain embodiments, the amide coupling agent is used with both a co-reagent such as HOBT or DMAP and a base such as triethylamine or diisopropylethylamine. One skilled in the art will recognize that co-reagents other than HOBT or DMAP may be used. Furthermore, one skilled in the art will recognize that bases other than triethylamine or diisopropylethylamine may be used.

In another specific embodiment, the present invention provides a method for producing a carrier represented by formula VII:

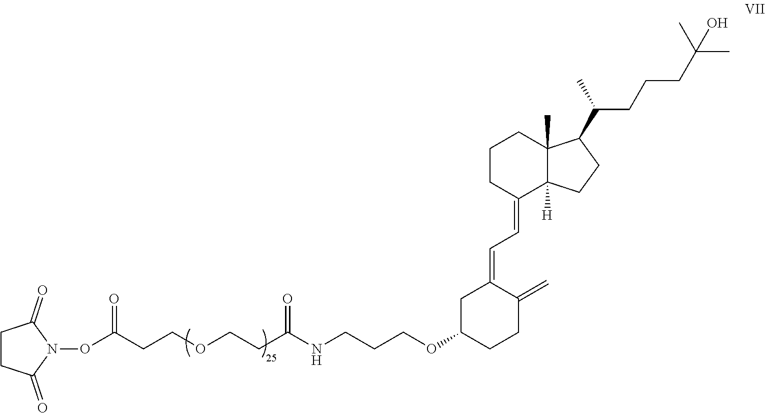

comprising the steps of reacting a compound of formula Vd:

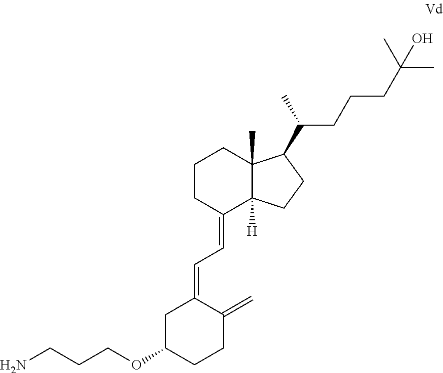 [SMILES: C=C1CC[C@H](OCCCN)C/C1=C/C=C1\CCC[C@]2(C)[C@@H]([C@H](C)CCCC(C)(C)O)CC[C@@H]12]

with a compound of formula VIIa:

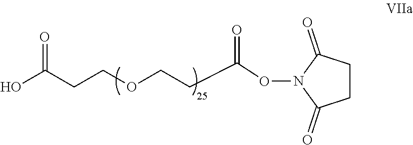

forming a compound of formula VIIb; and

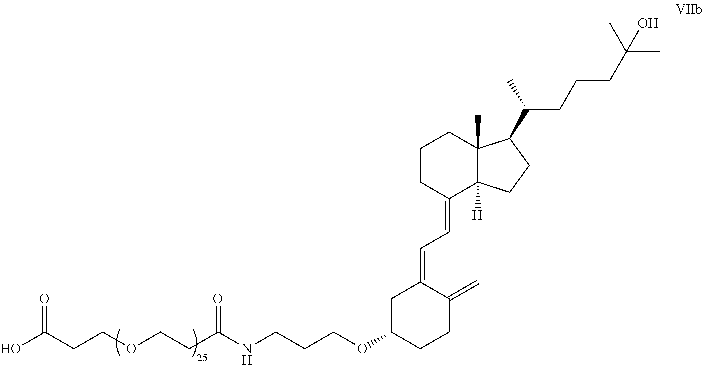

Converting a carboxylic acid of formula VIIb to an active ester of formula VII;

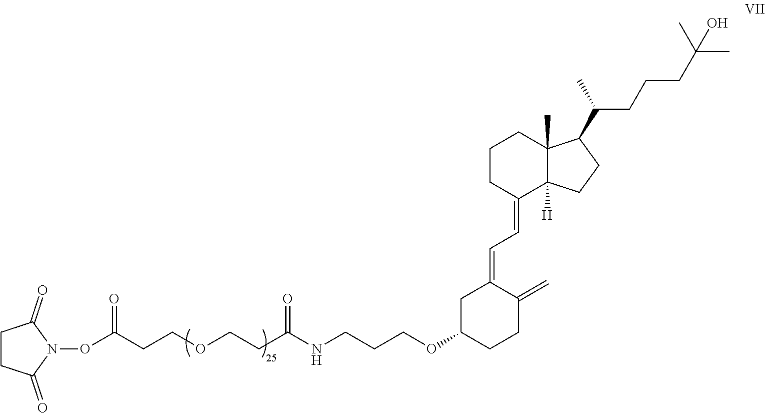

One skilled in the art will recognize that a compound of formula Vd can be used either as a free base or as a suitable salt form. Suitable salt forms include, but are not limited to TFA, HCl, HBr, MsOH, TfOH and AcOH.

In certain embodiments, a base such as triethylamine or diisopropylethylamine is used to promote coupling of the NETS-ester of formula VIIa with the amine of formula Va. One skilled in the art will recognize that bases other than triethylamine or diisopropylethylamine may be used.

NHS can be coupled with a carboxylic acid of formula VIIb in the presence of a suitable coupling reagent to form an active ester of formula VII. Suitable coupling reagents include, but are not limited to 2-chloromethylpyridinium iodide, BOP, PyBOP, HBTU, HATU, DCC, EDCI, TBTU, and T3P.

In some embodiments, an active ester of formula VII is formed from a carboxylic acid of formula VIIb using a combination of NHS and a coupling reagent.

In some embodiments, an active ester of formula VII is formed from a carboxylic acid of formula VIIb using a single reagent that produces a leaving group and also effects a coupling reaction. Such reagents include, but are not limited to, N,N′-disuccinimidyl carbonate. In some embodiments, the single reagent is used alone. In other embodiments the reagent is used with an acyl transfer catalyst. Such acyl transfer catalysts include, but are not limited to DMAP and pyridine. One skilled in the art will recognize that additional acyl transfer catalysts may be used.

One skilled in the art will recognize that there are other methods to conjugate a linker and scaffold to the C3 position of vitamin D derivatives and analogues. For example, the C₃ hydroxy group may be acylated by various groups as practiced by N. Kobayashi, K. Ueda, J. Kitahori, and K. Shimada, Steroids, 57, 488-493 (1992); J. G Haddad, et al., Biochemistry, 31, 7174-7181 (1992); A. Kutner, R. P. Link, H. K. Schnoes, H. F. DeLuca, Bioorg. Chem., 14, 134-147 (1986); and R. Ray, S. A. Holick, N. Hanafin, and M. F. Holick, Biochemistry, 25, 4729-4733 (1986).

The foregoing references are incorporated by reference in their entirety. One skilled in the art will recognize that these chemistries could be modified to synthesize compounds of the formula I:

B-(L)^{a}-S-(M)^{b}-C I

wherein B, S, C, (L)^{a}, and (M)^{b} are defined as above.

If desired, therapeutic compound carrier conjugates having different molecular weights can be isolated using gel filtration chromatography and/or ion exchange chromatography. Gel filtration chromatography may be used to fractionate different therapeutic compound carrier conjugates (e.g., 1-mer, 2-mer, 3-mer, and so forth, wherein “1-mer” indicates one targeting group molecule per therapeutic compound, “2-mer” indicates two targeting groups attached to therapeutic compound, and so on) on the basis of their differing molecular weights (where the difference corresponds essentially to the average molecular weight of the targeting group).

Gel filtration columns suitable for carrying out this type of separation include Superdex and Sephadex columns available from Amersham Biosciences (Piscataway, N.J.). Selection of a particular column will depend upon the desired fractionation range desired. Elution is generally carried out using a suitable buffer, such as phosphate, acetate, or the like. The collected fractions may be analyzed by a number of different methods, for example, (i) optical density (OD) at 280 nm for protein content, (ii) bovine serum albumin (BSA) protein analysis methods, for example, (i) optical density (OD) at 280 nm for protein content, (ii) bovine serum albumin (BSA) protein analysis, and (iii) sodium dodecyl sulfate polyacrylamide gel electrophoresis (SDS PAGE).

Separation of therapeutic compound carrier conjugates can also be carried out by reverse phase chromatography using a reverse phase-high performance liquid chromatography (RP-HPLC) C18 column (Amersham Biosciences or Vydac) or by ion exchange chromatography using an ion exchange column, e.g., a DEAE- or CM-Sepharose ion exchange column available from Amersham Biosciences. The resulting purified compositions are preferably substantially free of the non-targeting group-conjugated therapeutic compound. In addition, the compositions preferably are substantially free of all other non-covalently attached targeting groups

The invention provides compositions and methods for rendering a drug more potent by improving its pharmacokinetic properties using vitamin D or another DBP binding molecule. The natural pathway for the formation of vitamin D at the skin upon exposure to ultraviolet light relies on the interaction with DBP to bring the UV activated vitamin D into circulation where it can be utilized for cellular processes (Lips, Prog. Biophys. Molec. Biol. 92:4-8 (2006); DeLuca, Nutr. Rev. 66 (suppl. 2):573-578 (2008)). DBP brings vitamin D into circulation quickly and effectively. DBP also keeps active vitamin D in circulation for, on average, 30 days (Cooke, N. E., and J. G. Haddad. 1989. Endocr. Rev. 10:294-307; Haddad, J. G. et al. 1993. J. Clin. Invest. 91:2552-2555; Haddad, J. G. 1995. J. Steroid Biochem. Molec. Biol. 53:579-582). The invention provides for the first time using DBP to more effectively deliver therapeutic compounds such as G-CSF or compounds having G-CSF activity or GM-CSF or compounds having GM-CSF activity singly or in combination to the body. In one embodiment, the therapeutic compound is covalently linked or fused to a carrier. In another embodiment, the therapeutic compound is formulated with the carrier but not covalently linked. In one embodiment, the carrier interacts with DBP for the purpose of carrying the drug into the body more effectively from the site of administration. In another embodiment, the carrier keeps the drug in circulation for an extended period of time.

In one embodiment, the carrier comprises a targeting group and a coupling group for attaching the targeting group to the therapeutic compound. In another embodiment, the carrier comprises a scaffold moiety that is linked to the targeting group and the therapeutic compound. The targeting group is vitamin D, a vitamin D analog, a vitamin D-related metabolite, a vitamin D-related metabolite analog, or another molecule that can bind to or interact with the vitamin D binding protein (DBP). In one embodiment, the targeting group is an antibody or antibody derivative, a peptide designed to bind DBP or a fragment thereof, a peptide derived from a phage display or other peptide library selected against DBP or a fragment thereof, a nucleotide aptamer that binds DBP, a small molecule designed to bind DBP or derived from a chemical library selected against DBP, or a fragment thereof or moiety that can bind DBP as disclosed herein. In another embodiment, the carrier comprises DBP itself or a derivative of DBP.

Vitamin D is a group of fat-soluble secosteroids. Several forms (vitamers) of vitamin D exist. The two major forms are vitamin D₂ or ergocalciferol, and vitamin D₃ or cholecalciferol, vitamin D without a subscript refers to either D₂ or D₃ or both. In humans, vitamin D can be ingested as cholecalciferol (vitamin D₃) or ergocalciferol (vitamin D₂). Additionally, humans can synthesize it from cholesterol when sun exposure is adequate.

Vitamin D is further modified by enzymes found in various organs to a family of “vitamin D metabolites” that are also capable of binding DBP. For instance, vitamin D is converted to calcidiol (25OH hydroxy-Vitamin D) in the liver. Part of the calcidiol is converted by the kidneys to calcitriol (1,25 (OH)₂ dihydroxy-Vitamin D). Calcidiol is also converted to calcitriol outside of the kidneys for other purposes. Also found in the body is 24,25(OH)₂ dihydroxy-Vitamin D. Thus, in one embodiment, the targeting group is a vitamin D metabolite. In another embodiment, the targeting group is a “Vitamin D analog.” These compounds are based on the vitamin D structure and retain partial function of vitamin D. They interact with some of the same proteins as Vitamin D (e.g. DBP and the Vitamin D receptor), albeit at varying affinities. Exemplary analogs include: OCT, a chemically synthesized analogue of 1,25(OH)₂D3 with an oxygen atom at the 22 position in the side chain (Abe et. al., FEBS Lett. 226:58-62 (1987)); Gemini vitamin D analog, 1α,25-dihydroxy-20R-21(3-hydroxy-3-deuteromethyl-4,4,4-trideuterobutyl)-23-yne-26,27-hexafluoro-cholecalciferol (BXL0124) (So et al., Mol Pharmacol. 79(3):360-7 (2011)); Paricalcitol, a vitamin D₂ derived sterol lacking the carbon-19 methylene group found in all natural vitamin D metabolites (Slatopolsky et al., Am J Kidney Dis. 26: 852 (1995)); Doxercalciferol (1α-hydroxyvitamin D₂), like alfacalcidol (1α-hydroxyvitamin D₃), is a prodrug which is hydroxylated in the liver to 1α,25(OH)₂D2. Unlike alfacalcidol, doxercalciferol is also 24-hydroxylated to produce 1α,24(S) (OH)₂D2 (Knutson et al., Biochem Pharmacol 53: 829 (1997)); Dihydrotachysterol₂ (DHT₂), hydroxylated in vivo to 25(OH)DHT₂ and 1,25(OH)₂DHT₂ (McIntyre et al., Kidney Int. 55: 500 (1999)). See also Erben and Musculoskel, Neuron Interact. 2(1):59-69 (2001) and Steddon et al. Nephrol. Dial. Transplant. 16 (10): 1965-1967 (2001). The foregoing references are incorporated by reference in their entirety

As described herein, the carriers of the invention may be non-hormonal 25-hydroxy vitamin D or analogs thereof having a coupling group on the 3′ carbon. “25-hydroxy vitamin D analogs” as used herein includes both naturally-occurring vitamin D metabolite forms as well as other chemically-modified forms. The carriers of the invention do not include an active (i.e. hormonal) form of vitamin D (typically having a hydroxyl group at the 1 carbon). These compounds are based on the vitamin D structure and retain partial function of vitamin D (i.e. they interact with DBP), albeit at varying affinities. The following list exemplifies vitamin D analog forms known in the art. They may, however, be hormonal or have the C₁ hydroxyl group. They are presented here solely for their chemical properties as vitamin D analogs, not for their functional hormonal properties: OCT, a chemically synthesized version of 1,25(OH)2D3 with an oxygen atom at the 22 position in the side chain (Abe et. al., FEBS Lett. 226:58-62 (1987)); Gemini vitamin D analog, 1α,25-dihydroxy-20R-21(3-hydroxy-3-deuteromethyl-4,4,4-trideuterobutyl)-23-yne-26,27-hexafluoro-cholecalciferol (BXL0124) (So et al., Mol Pharmacol. 79(3):360-7 (2011)); Paricalcitol, a vitamin D2 derived sterol lacking the carbon-19 methylene group found in all natural vitamin D metabolites (Slatopolsky et al., Am J. Kidney Dis. 26: 852 (1995)); Doxercalciferol (1α-hydroxyvitamin D₂), like alfacalcidol (1α-hydroxyvitamin D₃), is a prodrug which is hydroxylated in the liver to 1α,25(OH)₂D₂, however, unlike alfacalcidol, doxercalciferol is also 24-hydroxylated to produce 1α,24(S) (OH)₂D₂ (Knutson et al., Biochem Pharmacol 53: 829 (1997)); Dihydrotachysterols (DHT₂), hydroxylated in vivo to 25(OH)DHT₂, 1,25(OH)₂DHT₂ (McIntyre et al., Kidney Int. 55: 500 (1999)), ED-71, and eldecalcitol. See also Erben and Musculoskel, Neuron Interact. 2(1):59-69 (2001) and Steddon et al. Nephrol. Dial. Transplant. 16 (10): 1965-1967 (2001). The foregoing references are incorporated by reference in their entirety.

In another embodiment, the carrier further comprises a pharmaceutically acceptable scaffold moiety covalently attached to the targeting group and the therapeutic compound. The scaffold moiety of the carriers of the invention does not necessarily participate in but may contribute to the function or improve the pharmacokinetic properties of the therapeutic compound. The scaffolds of the invention do not substantially interfere with the binding of the targeting group to DBP. Likewise, the scaffolds of the invention do not substantially interfere with structure or function of the therapeutic compound. The length of the scaffold moiety is dependent upon the character of the targeting group and the therapeutic compound. One skilled in the art will recognize that various combinations of atoms provide for variable length molecules based upon known distances between various bonds (Morrison, and Boyd, Organic Chemistry, 3rd Ed, Allyn and Bacon, Inc., Boston, Mass. (1977), incorporated herein by reference). Other scaffolds contemplated by the invention include peptide linkers, protein linkers such as human serum albumin, an antibody or fragment thereof, nucleic acid linkers, small carbon chain linkers, carbon linkers with oxygen or nitrogen interspersed, also combinations of these examples are contemplated.

In another embodiment, the carrier further comprises a pharmaceutically acceptable scaffold moiety covalently attached to the targeting group and the therapeutic compound. The scaffold moiety of the carriers of the invention does not necessarily participate in but may contribute to the function or improve the pharmacokinetic properties of the therapeutic compound. The scaffolds of the invention do not substantially interfere with the binding of the targeting group to DBP. Likewise, the scaffolds of the invention do not substantially interfere with structure or function of the therapeutic compound. The length of the scaffold moiety is dependent upon the character of the targeting group and the therapeutic compound. One skilled in the art will recognize that various combinations of atoms provide for variable length molecules based upon known distances between various bonds (Morrison, and Boyd, Organic Chemistry, 3rd Ed, Allyn and Bacon, Inc., Boston, Mass. (1977), incorporated herein by reference). Other scaffolds contemplated by the invention include peptide linkers, protein linkers such as human serum albumin or immunoglobulin family proteins or fragments thereof, nucleic acid linkers, small carbon chain linkers, carbon linkers with oxygen or nitrogen interspersed, or combinations thereof. In preferred embodiments, the linkers are non-releasable or stable

In further embodiments of the invention, the therapeutic compounds defined and/or disclosed herein may be chemically coupled to biotin. The biotin/therapeutic compound can then bind to avidin.

In another embodiment, the drug is a small molecule or chemical entity such as G-CSF or other compound having G-CSF activity or GM-CSf or other compound having GM-CSF activity singly or in combination.

In another embodiment, the drug is G-CSF or other compound having G-CSF activity, whether PEGylated, glycosylated or otherwise covalently or noncovalently modified or left unmodified. Both G-CSF and biosimilars or interchangeables thereto and PegG-CSF and its biosimilars or interchangeables, as well as compounds having G-CSF activity are included.

In another embodiment, the drug is GM-CSF or other compound having GM-CSF activity, whether PEGylated, glycosylated or otherwise covalently or noncovalently modified or left unmodified. Both GM-CSF and biosimilars or interchangeables thereto and PegG-CSF and its biosimilars or interchangeables, as well as compounds having G-CSF activity are included.

G-CSF and GM-CS and other compounds having G-CSF or GM-CSG activity comprise a highly critical class of therapeutic molecules. As described by Mehta, et al., J Immunol. 2015 Aug. 15; 194(4): 1341-1349:

As of 2015 more than 20 million people have been treated with these drugs worldwide and annual sales in the US alone exceeded $5MM.

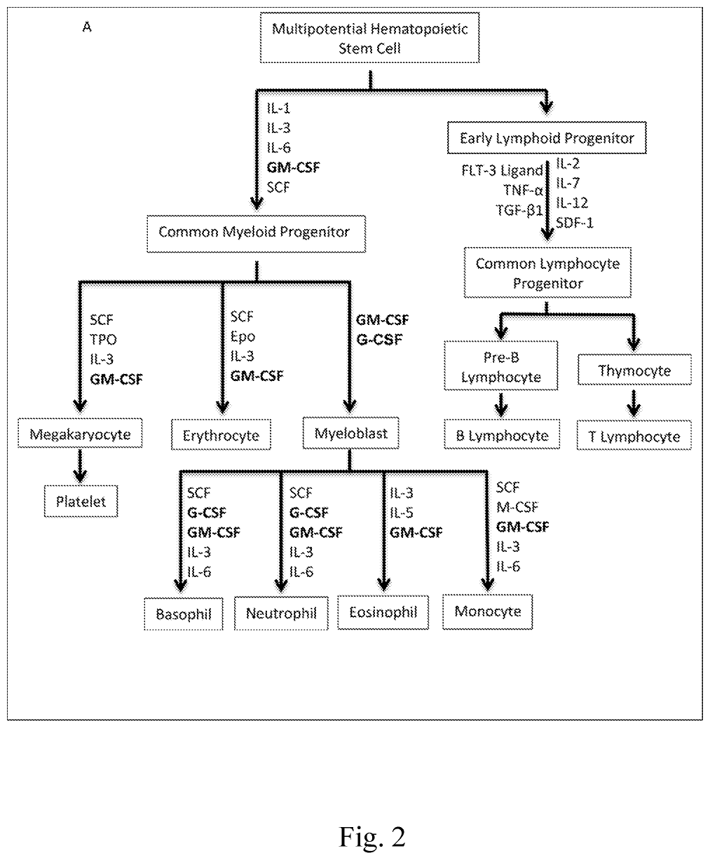

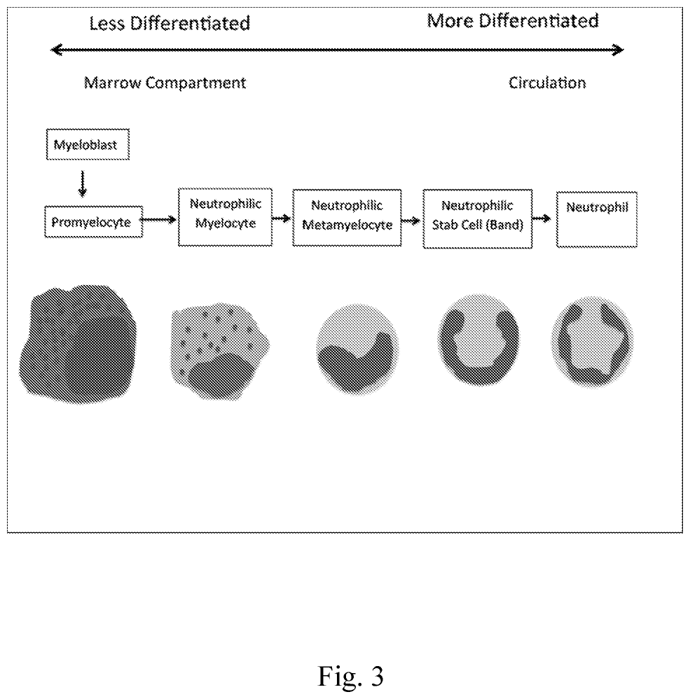

Hematopoiesis is a highly proliferative (˜10¹⁰ cells/day), dynamic process driven by multiple hematopoietic growth factors/cytokines (FIG. 2). The hematopoietic growth factors are multi-functional: critical for proliferation, survival, and differentiation of hematopoietic stem, progenitor, and precursor cells to a terminally differentiated, functional cell type. Colony forming assays identified the ability of first crude supernatants, then highly purified cytokines to drive muti-lineage and single lineage differentiation. After co-culturing for 7-14 days, colonies from mononuclear cells obtained from the mouse spleen or bone marrow were measured in semisolid medium. Based on characteristics of cells within a single colony, the lineage(s) governed by the cytokine was determined. Granulocytes comprise the majority of white blood cells in human circulation and play an integral role in innate and adaptive immunity. In granulopoiesis, their production is mediated by a number of different growth factors, especially G-CSF and GM-CSF. Due to asymmetric division some daughter cells of the hematopoietic stem cell (HSC) remain as HSC, preventing the depletion of the stem cell pool. Multiparameter immunophenotyping has transformed our ability to identify different cell types in hematopoiesis. Murine HSC are characterized as lin⁻sca1^{+c-}kit⁺, and human HSC display CD34⁺ in the absence of lineage markers. The differentiation pathway from HSC to granulocytes is dependent on G-CSF and, less so, GM-CSF. The HSC gives rise to a common myeloid progenitor (CMP) and common lymphoid progenitor (CLP) cell. The CMP cells differentiate into myeloblasts, erythrocytes, and megakaryocytes via at least two intermediates, the Granulocyte/Monocyte Progenitor cell and the Erythrocyte/Megakaryocyte Progenitor cell. In the granulocytic series, myeloblasts (15-20 μm) are the first recognizable cells by their scant cytoplasm, absence of granules, and fine nucleus with nucleoli in the bone marrow clearly committed to differentiation to granulocytes. Myeloblasts differentiate into promyelocytes, which are larger (20 μm) and begin to possess granules (See FIG. 3). Promyelocytes give rise to neutrophilic, basophilic, and eosinophilic precursor cells. Cell division continues through the promyelocyte stage. Fine specific granules containing inflammatory-related proteins appear during myelocyte maturation. For neutrophils, their size an nuclei become increasingly more condensed as the cells mature through myelocyte, metamyelocyte, band, and the terminally differentiated neutrophil (polymorphonuclear and ˜15 μm). During episodes of stress such as infection, band cells can be found in the peripheral blood and are used as a measure of inflammation. The above process is a complex and dynamic process orchestrated by multiple cytokines and their receptors, most notably G-CSF and GM-CSF.

Following antigen stimulation or activation by cytokines such as IL-1, IL-6, and TNFα, macrophages, T cells, endothelial cells, and fibroblasts produce and secrete G-CSF and GM-CSF. Of unknown significance, a variety of tumor cells also produce these paracrine growth factors. Glycoproteins with a molecular weight of ˜23 kDa, G-CSF and GM-CSF are now produced through recombinant technology in either E. coli or yeast. G-CSF induces the appearance of colonies containing only granulocytes, while GM-CSF gave colonies containing both granulocytes and macrophages. Generation of G-CSF (genomic nomenclature: Csf3) and G-CSF Receptor (Csf3r) knockout mice confirmed that G-CSF critically drives granulopoiesis. The cognate receptor for G-CSF is a single transmembrane receptor that homodimerizes upon G-CSF binding. Unlike G-CSF, GM-CSF functions via a two receptor system involving a specific α-chain and a common β-chain shared by IL-3 and IL-5. GM-CSF knockout mice however did not display a perturbation in hematopoiesis. Both G-CSF and GM-CSF signal through pathways involving JAK/STAT, SRC family kinases, PI3K/AKT, and Ras/ERK1/2. The receptor complexes are characterized by high-affinity (apparent Kd˜100-500 μM) and low density (50-1000 copies/cell). Interestingly, human G-CSF is functionally active on murine myeloid cells, but human GM-CSF is not. The signaling specificity likely involves nuances in the proximal post-receptor phosphoprotein networks and the distal gene regulatory networks. The molecular pathways and their cross-interactions in determining lineage specificity are critical to development of more specific therapies.

Cloning of human GM-CSF and its expression in bacterial and eukaryotic cells was achieved in 1985 at Genetics Institute, and, a year later, G-CSF and its expression in E. coli at Amgen. Commercialized by these biotechnology start-ups, G-CSF and GM-CSF revolutionized the treatment of patients with congenital or acquired neutropenias and those undergoing stem cell transplantation. Sidelined from the treatment of neutropenias by its toxicity profile, GM-CSF is now undergoing a renaissance as an immunomodulatory agent.

G-CSF is approved by the United States Food and Drug Administration (FDA) for use to decrease the incidence of infection in patients with non-myeloid malignancies receiving myelosuppressive anti-cancer drugs associated with a significant incidence of severe neutropenia with fever; reduce the time to neutrophil recovery and the duration of fever, following induction or consolidation chemotherapy treatment of patients with (AML) leukemia; reduce the duration of neutropenia and febrile neutropenia in patients with non-myeloid malignancies undergoing myeloablative chemotherapy followed by stem cell transplantation; mobilize hematopoietic progenitor cells into the peripheral blood for collection by leukapheresis; and reduce the incidence and duration of complications of severe neutropenia in symptomatic patients with congenital neutropenia, cyclic neutropenia, or idiopathic neutropenia. Forms of G-CSF available worldwide include filgrastim, pegfilgrastim, and lenograstim.

GM-CSF is currently approved by the FDA to accelerate myeloid recovery in patients with non-Hodgkin's lymphoma, acute lymphoblastic leukemia, and Hodgkin's disease undergoing autologous stem cell transplantation; following induction chemotherapy in older adult patients with AML to shorten time to neutrophil recovery and reduce the incidence of life-threatening infections; to accelerate myeloid recovery in patients undergoing allogeneic stem cell transplantation from HLA-matched related donors; for patients who have undergone allogeneic or autologous stem cell transplantation in whom engraftment is delayed or failed; and to mobilize hematopoietic progenitor cells into peripheral blood for collection by leukapheresis. Forms of GM-CSF available worldwide are sargramostim and molgramostim.

The recommended dosage of G-CSF is 5 mcg/kg/day, and for GM-CSF, 250 mcg/m²/day. Both drugs may be given subcutaneously or intravenously, although randomized clinical trials demonstrate greater efficacy (i.e., decreased duration of neutropenia) without a difference in toxicity for the subcutaneous route (13). For chemotherapy-induced neutropenia, G-CSF is administered until there is >1000 neutrophils/μl. For congenital neutropenias, the goal is to maintain neutrophil counts ˜750/μl. G-CSF is well tolerated. Transient fever and bone pain are more commonly observed in those receiving GM-CSF. Pleural and/or pericardial effusions can also occur in those receiving GM-CSF. Long-term side effects, such as osteopenia, of G-CSF administration are being monitored in patients with severe congenital neutropenia (SCN). One concern is that G-CSF may accelerate the transformation of SCN to myelodysplastic syndromes (MDS) or AML, associated with acquired mutations in the G-CSF Receptor.

The receptors for both GM-CSF and G-CSF belong to the hematopoietin/cytokine receptor superfamily. The G-CSF Receptor (G-CSFR) acts as a homodimer, whereas the GM-CSF Receptor is a heterodimer with a shared p chain with the IL-3 Receptor and IL-5 Receptor complexes. The G-CSFR is expressed primarily on neutrophils and bone marrow precursor cells, which undergo proliferation and eventually differentiation into mature granulocytes. G-CSF binds to G-CSFR, resulting in its dimerization, with a stoichiometry of 2:2 and with a high affinity (K_{D}=500 μM). Among the activated downstream signal transduction pathways are Janus kinase (JAK)/signal transducer and activator of transcription (STAT), Src kinases such as Lyn, Ras/Extracellular Regulated Kinase (ERK), and phosphatidylinositol 3-kinase (PI3K)(16). The cytoplasmic domain of G-CSFR possesses four tyrosine residues (Y704, Y729, Y744, Y764), serving as phospho-acceptor sites. Src homology 2 (SH2) containing proteins STAT5 and STAT 3 bind to Y704 and Gab2 to Y764 . . . Grb2 couples to both Gab2 and to SOS, permitting signaling diversification, such as Ras/ERK, PI 3-kinase/Akt, and Shp2 (, 20). An alternatively spliced isoform of G-CSFR elicits activation of a JAK-SHP2 pathway(15). The precise physiological roles of protein kinases and their downstream events in G-CSF-induced signaling remain unclear, although some clues are beginning to emerge (21, 22).

GM-CSF binds to the α chain of the GM-CSFR with a low affinity (K_{D}=0.2-100 nM), but a higher affinity (K_{D}=100 μM) occurs in the presence of both subunits. GM-CSF signaling involves formation of dodecameric supercomplex that is required for JAK activation (23). In addition to JAK/STAT pathway, GM-CSF also activates the ERK1/2, PI3K/Akt and IκB/NFκB pathways. Although the α-chain is considered primarily as ligand recognition units, it interacts with Lyn to activate JAK independent Akt activation of the survival pathway (24). Thus, differences in receptor expression patterns and known and unknown nuances in signaling pathway circuits account for the functional differences between G-CSF and GM-CSF.

G-CSF and GM-CSF are pleiotropic growth factors, with overlapping functions. GM-CSF also shares properties with and macrophage colony stimulating factor (M-CSF) on monocyte function. (25) Both GM-CSF and G-CSF increase chemotaxis and migration of neutrophils, but response kinetics may differ. GM-CSF may be considered to be more pro-inflammatory than G-CSF. As GM-CSF increases cytotoxic killing of C. albicans, surface expression of Fc- and complement-mediated cell-binding (FcγR1, CR-1 and CR-3), and adhesion receptor (ICAM-1) (14). Yet, both cytokines will promote neutrophil phagocytosis (26). More extensive reviews on G-CSF and GM-CSF function in neutrophils may be found (27, 28).

As further described by Mehta, et a., G-CSF remediates many forms of neutropenia:

An absolute neutrophil count (ANC) less than 1,500/μl is defined as neutropenia, which is graded on the severity of decreased ANC (Table 2). Causes for neutropenia may be congenital or, more commonly, acquired. Neutropenia may be asymptomatic until an infection occurs. Benign neutropenia exists, and the individuals are not at risk for serious infection. However, onset of fever with neutropenia, termed febrile neutropenia, commonly occurs as a potentially life-threatening complication of chemotherapy and involves considerable cost due to treatment with intravenous antibiotics and prolonged hospitalization. In addition, febrile neutropenia prevents continuation of chemotherapy until there is recovery from neutropenia. According to the Norton-Simon hypothesis(29), the efficacy of chemotherapy would be reduced if stopped midway. A pause in treatment allows recovery of the cancer cells and facilitates the emergence of chemoresistant clones(29-31). Neutropenia also occurs secondary to bone marrow infiltration with leukemic or myelodysplastic cells.

TABLE 2
Correlation of neutropenia with absolute neutrophil count

| Neutropenia Grade | Absolute neutrophil count |
|---|---|
| Grade 1 | ≥1.5 × 10⁹/ml −< 2 × 10⁹/ml |
| Grade 2 | ≥1 × 10⁹/ml −< 1.5 × 10⁹/ml |
| Grade 3 | ≥0.5 × 10⁹/ml −< 1 × 10⁹/ml |
| Grade 4 | <0.5 × 10⁹/ml |

Neutropenia results from a growing list of germline mutations in genes, such as ELANE, HAX1, GFI1, G6PC3, WAS, and CSF3R. Soon after birth, children with SCN develop a grade 4 neutropenia. SCN is a lifetime condition resulting from increased apoptosis of granulocytic progenitors in the marrow. Due to the severity and chronic nature of SCN, individuals are prone to recurrent infections, especially from the endogenous flora in the gut, mouth and skin. Most cases of SCN are due to de novo mutations. Transmission may be autosomal dominant, recessive, or X-linked. The most common mutation involves ELANE and is autosomal dominant. Mutations in ELANE encode the neutrophil elastase (NE), a serine protease. ELANE is expressed during ganulopoiesis, maximally at the promyelocyte stage. It is hypothesized that mutations in ELANE cause neutropenia via improper folding of the protein that triggers the unfolded protein response (UPR). UPR-generated stress drives apoptosis due to an overload of unfolded protein, and an arrest in differentiation at the promyelocyte stage is observed. Fascinatingly, ELANE mutations are also associated with cyclic neutropenia. Cyclic neutropenia is characterized by granulocyte nadirs of less than 200/μl occurring every 21 days.

Patients with SCN are always at risk for life-threatening infections. Early phase 1 clinical trials held in 1989 evaluated G-CSF therapy for SCN and cyclic neutropenia. Both trials demonstrated at least a 10-fold increase in neutrophil counts, reducing the severity of the neutropenia from grade 4 to grade 1 to normal counts. Reduction in days of cyclic neutropenia from 21 to 14 days was observed and in SCN a consistent increase in ANC was observed. In 1990 two studies explored the benefit of G-CSF versus GM-CSF in treating congenital neutropenia. Grey collie dogs with cyclic neutropenia due to mutations in the endocytosis gene AP3B1 were studied with three cytokines, G-CSF, GM-CSF and TL-3. GM-CSF and G-CSF showed an expansion of neutrophil counts, but only G-CSF prevented the cycling of hematopoiesis. Similar to the dog study, G-CSF therapy increased ANC, whereas GM-CSF therapy increased eosinophil counts, but not neutrophil counts. Following the beneficial effects of G-CSF in the above phase ½ studies, a phase 3 clinical trial was performed in 1993. Patients with SCN, cyclic neutropenia, and idiopathic neutropenia (n=123) were included in the study. Patients were randomly treated immediately or after a four-month observation period. Almost all of the patients (108 of 120) receiving G-CSF therapy displayed a restoration of ANC from grade 4 to normal levels. The increase in ANC resulted from increased production of neutrophils in bone marrow. Infection related incidents were reduced by ˜50% (P<0.05) and a reduction by 70% in antibiotic use.

One particular form of inherited neutropenia is the WHIM (warts, hypogammaglobulinemia, infections, and myelokathexis) syndrome. Myelokathexis refers to a build-up of mature neutrophils in the bone marrow. Mutations in CXCR4 result in the syndrome. CXCR4 and its ligand SDF-1 mediate the retention of neutrophils. G-CSF administration leads to upregulation of SDF-1 and subsequent release of neutrophils into the peripheral circulation. A recently published phase I study demonstrated the safety and efficacy of low-dose plerixafor, a CXCR4 antagonist. One widely-used indication for G-CSF to mobilize and harvest hematopoietic progenitor cells into the periphery for stem cell transplantation, and concomitant use of plerixafor enhances the mobilization.

Severe aplastic anemia (SAA) is a disease where stem cells residing in the bone marrow are damaged leading to a deficiency in all hematopoietic cell lines. SAA has a high mortality rate, but the five-year mortality rate is reduced to less than 10% with matched sibling stem cell transplantation or 30% with immunosuppressive therapy (IST). IST includes antithymocyte globulin, cyclosporine, and glucocorticoids. The addition of G-CSF to IST has been studied in a number of randomized studies and showed that G-CSF reduces the number of infectious complications and hospital days when compared to standard therapy alone. However, its addition did not affect a difference in overall survival rates. While treatment with G-CSF or GM-CSF results in a neutrophil response, a sustained tri-lineage response was uncommon when used alone or in combination with other hematopoietic growth factors. The response to G-CSF may have prognostic value. Patients treated with IST plus G-CSF who did not achieve a white blood cell count of at least 5,000/μl had a low probability of response and high mortality. Similarly GM-CSF has been studied as a potential adjunct to IST with similar results. These finding suggest G-CSF and GM-CSF may be useful adjuncts to standard IST for SAA.

The FDA approved in 1991 the use of recombinant human G-CSF (filgrastim) to treat cancer patients undergoing myelotoxic chemotherapy. Multiple factors affect the severity of neutropenia, most important being the type and severity of chemotherapy dosage and the underlying disease. In 1994 the American Society of Clinical Oncology (ASCO) recommended primary prophylaxis with G-CSF or GM-CSF for expected incidence of neutropenia of >40%. The purpose of the guidelines was to reduce the incidence and length of neutropenia and thus time of hospitalization, which would reduce costs significantly. Three prospective, randomized, placebo controlled trials formed the basis of the recommendations. The first phase 3 trial tested the applicability of G-CSF as an adjunct to chemotherapy in patients treated for small cell lung cancer with cyclophosphamide, doxorubicin, and etoposide (CDE). A major outcome of the study identified a significant reduction of at least one episode of febrile neutropenia occurring at 77% in placebo versus 40% in G-CSF group (P<0.001). A reduction in median duration of grade 4 neutropenia was observed in all cycles of chemotherapy (1-day G-CSF group versus 6-days placebo group). From a cost-benefit perspective, the data translated into reduction of 50% incidence of infection, antibiotic treatment, and days of hospitalization with G-CSF treatment versus placebo. A similar study performed in Europe for small cell lung cancer also found that prophylactic G-CSF treatment reduced the incidence of febrile neutropenia (53% placebo group versus 26% G-CSF group). A significant reduction in hospitalization and antibiotic treatment was observed. The study also identified a benefit of G-CSF treatment in adherence to the chemotherapy regimen. A reduction in chemotherapy dose by 15% was indicated in 61% of the placebo group versus 29% of the G-CSF group. A gap of two or more days in the chemotherapy treatment group was observed for 47% patients of the placebo group and 29% of G-CSF group. The third trial investigated G-CSF therapy in non-Hodgkin lymphoma (NHL) treated with vincristine, doxorubicin, prednisolone, etoposide, cyclophosphamide, and bleomycin (VAPEC-B). Incidence of neutropenia was reduced for the G-CSF group (23%) versus placebo group (44%), with fewer delays and shorter duration of treatment in G-CSF-treated group. In comparison, GM-CSF trials provided less convincing data. In a trial for cyclophosphamide, vincristine, procarbazine, bleomycin, prednisolone, doxorubicin, and mesna (COP-BLAM) administered as therapy for NHL, use of molgramostim (GM-CSF) resulted in faster recovery from neutropenia and reduced hospitalization, but the benefit was limited to only 72% of the patients that could tolerate GM-CSF. Another trial with small cell lung cancer did not show any significant effect with molgramostim treatment.

Development of better chemotherapeutic regimens that were less myelotoxic, provided more cost effective options compared to colony stimulating factor therapy. The incidence of neutropenia in many cases was reduced to <10%. However, the advantage of colony stimulating factor therapy in both increasing the intensity and maintenance of dose were actively debated versus the cost of the growth factors. The 2000 ASCO guidelines noted the lack of colony stimulating factor therapy in improving survival benefits with newer chemotherapeutic regimens. In 2003, a large randomized study showed benefit of G-CSF therapy for a dose-dense chemotherapy (cyclophosphamide, paclitaxel, and doxorubicin) in patients with node-positive breast cancer. Significantly improved disease-free survival (RR 0.74, p=0.01) and overall survival (RR 0.69, p=0.013) was observed in patients receiving G-CSF. Fewer patients reported grade 4 neutropenia in G-CSF group (6%) versus non-G-CSF group (33%). In 2004, two additional studies with old (60-75) and young (<60 years) NHL patients observed a reduction of chemotherapy regimens from 3 to 2 weeks combined with an improved the rate of progressive disease and overall survival. In 2005 two trials emerged that brought about significant support for G-CSF support and reduced the threshold for recommended CSF therapy from 40% to 20%. The first study compared the effect of antibiotics (A) versus antibiotics+G-CSF (A+G) in small cell lung cancer patients undergoing CDE treatment. A significant reduction in incidence of febrile neutropenia was observed for A+G group (10%) versus antibiotics only group (24%). The second study investigated effect of pegfilgrastim in breast cancer patients treated with docetaxel. Approved in 2001, pegfilgrastim was developed to improve the renal clearance rate and a single dose provided similar or greater improvement in the ANC after chemotherapy compared to daily filgrastim doses. The randomized, placebo-controlled trial conducted with 928 patients demonstrated a lower incidence of febrile neutropenia in patients receiving pegfligrastim (1%) versus placebo (17%). Hospitalization was also reduced in pegfilgrastim group (1%) versus placebo group (14%). In 2005 and 2006, the National Comprehensive Cancer Network (http://www.nccn.org) and ASCO adopted guidelines that reduced the threshold from 40% to 20% for the risk of neutropenia to be treated with growth factors as an adjuvant to chemotherapy. The issues of use of myeloid growth factors, their cost-effectiveness, and the duration of their use during chemotherapy remain of great interest to clinical oncologists. A randomized phase 3 study with a non-inferiority design demonstrated the efficacy of G-CSF prophylaxis against febrile neutropenia in women with breast cancer for the entirety of their myelosuppressive treatment. Current guidelines from American Society of Clinical Oncology, National Comprehensive Cancer Network, and the European Organisation for Research and Treatment of Cancer recommend the use of myeloid growth factors when the risk of febrile neutropenia is 20% or greater.

Neutropenia Associated with Leukemia

Neutropenia in patients with leukemia results from both the underlying disease and aggressive chemotherapy. The ASCO guidelines developed in 1994, like for solid tumors, considered data obtained from three large randomized trials. Unlike the solid tumor trials, two of the three trials used GM-CSF versus G-CSF. The two GM-CSF trials reported conflicting findings, with some statistical significance in recovery of ANC, but no significant reduction in hospitalization or incidence of serious infections. The G-CSF trial showed a recovery in ANC, reduction in days of neutropenia, and a trend towards better recovery rates. However, like the GM-CSF trials no improvement in days of hospitalization or usage of antibiotics was observed. Thus a beneficial response by the growth factors was not observed in case of leukemia at this time. However at the time of ASCO's 2000 guidelines, newer placebo-controlled trials demonstrated a reduction in neutrophil recovery time from 6 days to 2 days and reduced hospitalization times in the setting of induction chemotherapy. The 2000 ASCO guidelines also identified a potential benefit for growth factor therapy in consolidation chemotherapy. The 2006 update did not introduce any significant changes and recommended the application of CSF therapy post-induction and consolidation therapy.

Unlike chemotherapy-induced neutropenia, congenital neutropenia patients experience neutropenia for life and require long-term treatment with G-CSF. Long-term effects of G-CSF therapy have become important in management of congenital neutropenia. Patients receiving G-CSF therapy for as long as eight years were evaluated for safety and efficacy. Neutrophil counts were maintained without exhaustion of myelopoiesis. A significant improvement in the quality of life was achieved by reduction in antibiotic treatment and hospitalization time allowing for normal growth, development, and participation in normal daily activities. The SCN international registry (SCNIR) was formed in 1994 to further assess the progress of SCN patients being treated with G-CSF. A ten year report that followed patients with SCN (n=526) being treated with G-CSF was released in 2006. Consistent with previous reports, an increase with maintenance of ANC was observed in majority of the patients with an overall improvement in quality of life.

Leukemia transformation is significantly higher in SCN patients, and the SCNIR reported 21% of patients with SCN developed leukemia while being treated with G-CSF. Although leukemic transformation have been reported in SCN patients before the development of G-CSF therapy, the precise role of G-CSF therapy in leukemic transformation remains unknown. Almost all SCN patients undergo G-CSF therapy, and thus it is difficult to assess leukemic transformations in the absence of G-CSF treatment. However, patients who require higher doses of G-CSF are at a higher risk of developing MDS/AML.

Germline mutations in CSF3R, which encodes the G-CSFR, are infrequent causes for SCN, and result in refractoriness to filgrastim(81). Acquired nonsense mutations in CSF3R have been observed in ˜80% of SCN patients who progressed to secondary MDS/AML. The nonsense mutations result in deletion of the C-terminus of the G-CSFR, resulting in the loss of one to all four tyrosine residues and the inability to undergo normal ligand-induced internalization and endosomal routing. The truncated receptor mutants produce a phenotype of enhanced proliferation and impaired differentiation in response to G-CSF. Furthermore, knock-in mice harboring a similar mutation showed hyperproliferative responses to G-CSF administration and strongly prolonged activation of STAT5, implicated in increased hematopoietic progenitor stem cell expansion in vivo (89). This prediction was validated in a patient with SCN who developed secondary AML concomitant with a nonsense mutation of G-CSFR. Upon discontinuation of G-CSF and without chemotherapy, the blast count in the blood and bone marrow disappeared, although the mutation remained detectable. The tight correlation between the acquisition of G-CSFR mutations and progression of SCN to secondary MDS/AML and the abnormal signaling features in vitro and in vivostrongly suggested that mutated CSF3R could be a driver of myelodysplasia. Recent studies reveal that CSF3R T595I mutation is the most prevalent mutation found in chronic neutrophilic leukemia and that treatment with the Jak2 inhibitor ruxolitinib resulted in marked clinical improvement support the hypothesis that mutations in G-CSFR are indeed drivers of myeloproliferative disease. A low frequency of CSF3R mutations also occurs in AML and chronic myelomonocytic leukemia.

G-CSF and/or GM-CSF may improve chemotherapy and immunotherapy of hematologic malignancies and non-blood cancers. For instance, these myeloid growth factors can recruit quiescent leukemic cells into the cell cycle for enhanced killing from cell cycle-specific chemotherapy. As a pro-inflammatory cytokine, GM-CSF is being used to promote dendritic cell activity in a variety of anti-cancer trials. Indeed, GM-CSF is approved as part of the sipuleucel-T regimen for the treatment of hormone-resistant prostate cancer. There, dendritic cells are incubated with a fusion protein consisting of prostatic acid phosphatase and GM-CSF. While sipuleucel-T has been underused, in part due to its expense, GM-CSF is being studied in the context of other immunotherapeutic interventions (clinicaltrials.gov).

G-CSF has immunomodulatory effects on immune cells. G-CSF enhances antibody-dependent cellular cytotoxicity and cytokine production in neutrophils(98). However, it also inhibits Toll Like Receptor-induced pro-inflammatory cytokines produced by monocytes and macrophages. CD34+ monocytes that inhibit graft-versus-host disease are mobilized in response to G-CSF. In addition, G-CSF inhibits LPS-induced IL-12 production from bone-marrow derived dendritic cells cultured in vitro. Interestingly, administration of GM-CSF has the opposite effect, inducing cytokine production in the circulation in response to LPS.

GM-CSF pathways may be high-value targets in autoimmune diseases. For example, inflammatory bowel disease (IBD) is a chronic inflammatory condition of the gastrointestinal tract caused by a combination of environmental and genetic factors. Crohn's disease and ulcerative colitis can be difficult to treat and relapse of disease can occur at any time. Biochemical markers identifying patients at risk for relapse are currently lacking. GM-CSF signaling has recently been implicated in the pathogenesis of Crohn's disease. GM-CSF is required for myeloid cell antimicrobial functions and homeostatic responses to tissue injury in the intestine. Preliminary studies have found that GM-CSF reduces chemically-induced gut injury in mice. In human studies, higher concentrations of circulating antibodies against GM-CSF are found in patients with active IBD as compared with those with inactive disease. There are currently several studies and clinical trials looking at the use of GM-CSF in the treatment of IBD and anti-GM-CSF antibody for the monitoring of disease activity and assessing risk of recurrence.

Pulmonary alveolar proteinosis (PAP) is a rare disorder characterized by accumulation of periodic acid-schiff-positive lipoproteinaceous material in the alveoli of the lung leading to impaired gas exchange, respiratory insufficiency, and in severe cases, respiratory failure. Autoimmune PAP (aPAP) accounts for 90% of cases and is due to the presence of autoantibodies against GM-CSF. Hereditary PAP (hPAP) is caused by mutations in the genes CSF2RA and CSF2RB that code for the α and β subunit of the GM-CSF receptor respectively. In aPAP, the presence of anti-GM-CSF antibodies leads to aberrant in vivo GM-CSF signaling that is required for macrophage-mediated clearance, but not uptake, of pulmonary surfactant. This results in the progressive accumulation of foamy surfactant laden macrophages and intra-alveolar surfactant in the alveoli of the lung. The gold standard of therapy has been whole lung lavage. Although an effective therapy, it often needs to be repeated due to re-accumulation of lipoproteinaceous sediment and is not without complications. Newer therapies have been studied including pulmonary macrophage transplantation, plasmapheresis to remove the GM-CSF autoantibody, and inhaled GM-CSF. Inhaled GM-CSF is of particular interest as it has been shown in animal studies and phase I and II clinical trials to be safe and effective.

Some aspects of the assembly of carriers utilizes chemical methods that are well-known in the art. For example, Vitamin E-PEG is manufactured by Eastman Chemical, Biotin-PEG is manufactured by many PEG manufacturers such as Enzon, Nektar and NOF Corporation. Methods of producing PEG molecules with some vitamins and other therapeutic compounds linked to them follows these and other chemical methods known in the art. The attachment of PEG to an oligonucleotide or related molecule occurs, for example, as the PEG2-N-hydroxysuccinimide ester coupled to the oligonucleotide through the 5′ amine moiety. Several coupling methods are contemplated and include, for example, NHS coupling to amine groups such as a lysine residue on a peptide, maleimide coupling to sulfhydryl group such as on a cysteine residue, iodoacetyl coupling to a sulfhydryl group, pyridyldithiol coupling to a sulfhydryl group, hydrazide for coupling to a carbohydrate group, aldehyde for coupling to the N-terminus, or tetrafluorophenyl ester coupling that is known to react with primary or secondary amines. Other possible chemical coupling methods are known to those skilled in the art and can be substituted.

By way of example, conjugation using the coupling groups of the invention may be carried out using the compositions and methods described in WO93/012145 (Atassi et al.) and also see U.S. Pat. No. 7,803,777 (Defrees et al.), incorporated by reference herein in their entirety.

In one embodiment, carrier compounds may be covalently or noncovalently attached to the drug. In another embodiment, the carrier compounds are separate from the drugs but are mixed together at discrete concentrations so as to become formulated into functional units. Exemplary drug formulations of the invention include aqueous solutions, organic solutions, powder formulations, solid formulations and a mixed phase formulations.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts retain the desired biological activity of the therapeutic composition without toxic side effects. Examples of such salts are (a) acid addition salts formed with inorganic acids, for example, hydrochloric acid, hydrobromic acid, sulfuric acid, phosphoric acid, nitric acid and the like/and salts formed with organic acids such as, for example, acetic acid, trifluoroacetic acid, tartaric acid, succinic acid, maleic acid, fumaric acid, gluconic acid, citric acid, malic acid, ascorbic acid, benzoic acid, tanic acid, pamoic acid, alginic acid, polyglutamic acid, naphthalenesulfonic acid, naphthalene disulfonic acid, polygalacturonic acid and the like; (b) base addition salts or complexes formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethlenediamine; or (c) combinations of (a) and (b), e.g. a zinc tannate salt and the like.

The pharmaceutical compositions of this invention may be administered by transdermal, oral, parenteral, inhalation, ocular, topical, rectal, nasal, buccal (including sublingual), vaginal, or implanted reservoir modes. The pharmaceutical compositions of this invention may contain any conventional, non-toxic, pharmaceutically-acceptable carriers, adjuvants or vehicles. The term parenteral as used herein includes subcutaneous, intracutaneous, intravenous, intramuscular, intraarticular, intrasynovial, intrasternal, intrathecal, intralesional, and intracranial injection or infusion techniques.

Also contemplated, in some embodiments, are pharmaceutical compositions comprising as an active ingredient, therapeutic compounds described herein, or pharmaceutically acceptable salt thereof, in a mixture with a pharmaceutically acceptable, non-toxic component. As mentioned above, such compositions may be prepared for parenteral administration, particularly in the form of liquid solutions or suspensions; for oral or buccal administration, particularly in the form of tablets or capsules; for intranasal administration, particularly in the form of powders, nasal drops, evaporating solutions or aerosols; for inhalation, particularly in the form of liquid solutions or dry powders with excipients, defined broadly; for transdermal administration, particularly in the form of a skin patch or microneedle patch; and for rectal or vaginal administration, particularly in the form of a suppository.

The compositions may conveniently be administered in unit dosage form and may be prepared by any of the methods well-known in the pharmaceutical art, for example, as described in Remington's Pharmaceutical Sciences, 17^{th} ed., Mack Publishing Co., Easton, Pa. (1985), incorporated herein by reference in its entirety. Formulations for parenteral administration may contain as excipients sterile water or saline alkylene glycols such as propylene glycol, polyalkylene glycols such as polyethylene glycol, saccharides, oils of vegetable origin, hydrogenated napthalenes, serum albumin or other nanoparticles (as used in Abraxane™ American Pharmaceutical Partners, Inc. Schaumburg, Ill.), and the like. For oral administration, the formulation can be enhanced by the addition of bile salts or acylcarnitines. Formulations for nasal administration may be solid or solutions in evaporating solvents such as hydrofluorocarbons, and may contain excipients for stabilization, for example, saccharides, surfactants, submicron anhydrous alpha-lactose or dextran, or may be aqueous or oily solutions for use in the form of nasal drops or metered spray. For buccal administration, typical excipients include sugars, calcium stearate, magnesium stearate, pregelatinated starch, and the like.

Delivery of modified therapeutic compounds described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

In certain embodiments for transdermal administration, delivery across the barrier of the skin would be enhanced using electrodes (e.g. iontophoresis), electroporation, or the application of short, high-voltage electrical pulses to the skin, radiofrequencies, ultrasound (e.g. sonophoresis), microprojections (e.g. microneedles), jet injectors, thermal ablation, magnetophoresis, lasers, velocity, or photomechanical waves. The drug can be included in single-layer drug-in-adhesive, multi-layer drug-in-adhesive, reservoir, matrix, or vapor style patches, or could utilize patchless technology. Delivery across the barrier of the skin could also be enhanced using encapsulation, a skin lipid fluidizer, or a hollow or solid microstructured transdermal system (MTS, such as that manufactured by 3M), jet injectors. Additives to the formulation to aid in the passage of therapeutic compounds through the skin include prodrugs, chemicals, surfactants, cell penetrating peptides, permeation enhancers, encapsulation technologies, enzymes, enzyme inhibitors, gels, nanoparticles and peptide or protein chaperones.

One form of controlled-release formulation contains the therapeutic compound or its salt dispersed or encapsulated in a slowly degrading, non-toxic, non-antigenic polymer such as copoly(lactic/glycolic) acid, as described in the pioneering work of Kent et al., U.S. Pat. No. 4,675,189, incorporated by reference herein. The compounds, or their salts, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, JR Robinson ed., Marcel Dekker Inc., New York, 1978; and Controlled Release of Biologically Active Agents, R W Baker, John Wiley & Sons, New York, 1987. The foregoing are incorporated by reference in their entirety.

An additional form of controlled-release formulation comprises a solution of biodegradable polymer, such as copoly(lactic/glycolic acid) or block copolymers of lactic acid and PEG, is a bioacceptable solvent, which is injected subcutaneously or intramuscularly to achieve a depot formulation. Mixing of the therapeutic compounds described herein with such a polymeric formulation is suitable to achieve very long duration of action formulations.

When formulated for nasal administration, the absorption across the nasal mucous membrane may be further enhanced by surfactants, such as, for example, glycocholic acid, cholic acid, taurocholic acid, ethocholic acid, deoxycholic acid, chenodeoxycholic acid, dehdryocholic acid, glycodeoxycholic acid, cycledextrins and the like in an amount in the range of between about 0.1 and 15 weight percent, between about 0.5 and 4 weight percent, or about 2 weight percent. An additional class of absorption enhancers reported to exhibit greater efficacy with decreased irritation is the class of alkyl maltosides, such as tetradecylmaltoside (Arnold, J J et al., 2004, J Pharm Sci 93: 2205-13; Ahsan, F et al., 2001, Pharm Res 18:1742046) and references therein, all of which are hereby incorporated by reference.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topical transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When formulated for delivery by inhalation, a number of formulations offer advantages. Adsorption of the therapeutic compound to readily dispersed solids such as diketopiperazines (for example, Technosphere particles [Pfutzner, A and Forst, T, 2005, Expert Opin Drug Deliv 2:1097-1106] or similar structures gives a formulation that results in rapid initial uptake of the therapeutic compound. Lyophilized powders, especially glassy particles, containing the therapeutic compound and an excipient are useful for delivery to the lung with good bioavailability, for example, see Exubera® (inhaled insulin by Pfizer and Aventis Pharmaceuticals Inc.).

Dosage levels of between about 0.01 and about 100 mg/kg body weight per day, preferably 0.5 and about 50 mg/kg body weight per day of the active ingredient compound are useful in the prevention and treatment of disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

The carrier-drug conjugate, fusion or formulation provides advantages to the drug manufacturer and the patient over the unconjugated, unfused or unformulated drug. Specifically, the carrier-drug conjugate or formulation will be a more potent and longer lasting drug requiring smaller and less frequent dosing compared to the unconjugated, unfused or unformulated drug. This translates into lowered healthcare costs and a more convenient drug administration schedule for the patient. The carrier-drug conjugate or formulation can also influence the route of injection of a drug that is normally infused by intravenous injection to now be administered via subcutaneous injection or in a transdermal delivery system. The route of administration via subcutaneous injection or transdermal delivery is most favored because they can be self-administered by patients at home. This can improve patient compliance.

In yet another aspect of the invention, the levels of DBP can be increased as part of the carrier-drug therapy. It has been reported that estrogen can increase DBP levels (Speeckaert et al., Clinica Chimica Acta 371:33). It is contemplated here that levels of DBP can be increased by administration of estrogen for more effective delivery of carrier-drug conjugates.

In yet another aspect of the invention, it is contemplated that the carrier can be used to deliver drugs transdermally. Since DBP normally transports UV activated vitamin D at locations close to the surface of the skin, the use of a transdermal delivery system with the carrier becomes feasible.

The invention provides carrier-drug conjugates comprising a targeting group that is non-hormonal vitamin D, an analog, or metabolite thereof linked at the carbon 3 position to a therapeutic compound. In some embodiments, the non-hormonal vitamin D molecules are not hydroxylated at the carbon 1 position.

The carriers enhance the absorption, stability, half-life, duration of effect, potency, or bioavailability of the therapeutic compounds. Optionally, the carriers further comprise scaffolding moieties that are non-releasable such as PEG and others described in this disclosure.

Thus, the invention provides a carrier-drug conjugate comprising a targeting group that is a non-hormonal vitamin D, analog, or metabolite thereof conjugated to a therapeutic compound at the carbon 3 position of said non-hormonal vitamin D targeting group. In some embodiments, the non-hormonal vitamin D is not hydroxylated at the carbon 1 position. In preferred embodiments, the targeting group is conjugated to the therapeutic compound via a scaffold that is between about 100 and 200,000 Da and is selected from the group consisting of poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, and an additional therapeutic compound.

In another embodiment, the invention provides a pharmaceutical composition comprising a carrier-drug conjugate comprising a targeting group that is a non-hormonal vitamin D, analog, or metabolite thereof conjugated to a therapeutic compound at the carbon 3 position of the non-hormonal vitamin D targeting group via a scaffold. In a preferred embodiment, the carrier increases the absorption, bioavailability, or half-life of said therapeutic compound in circulation.

In another preferred embodiment, the non-hormonal vitamin D is not hydroxylated at the carbon 1 position. In another preferred embodiment of the pharmaceutical composition, the scaffold is selected from the group consisting of poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, and an additional therapeutic compound.

In another preferred embodiment, the therapeutic compound is G-CSF or compounds having G-CSF activity.

The invention provides a method of treating a patient in need of a therapeutic compound, comprising administering an effective amount of the pharmaceutical compositions described herein. In the preferred embodiment, the therapeutic compound is G-CSF or a compound having G-CSF activity or GM-CSF or a compound having GM-CSF activity singly or in combination. The invention provides pharmaceutical compositions for the manufacture of a medicament for the treatment of a patient in need of said medicament. The invention provides a method of manufacturing the pharmaceutical composition disclosed herein, comprising conjugating the targeting group and the therapeutic compound, wherein the conjugating step utilizes a coupling group. In preferred embodiments, the coupling group is selected from the group consisting of an amine-reactive group, a thiol-reactive group, a maleimide group, a thiol group, an aldehyde group, an NETS-ester group, a haloacetyl group, an iodoacetyl group, a bromoacetyl groups, a SMCC group, a sulfo SMCC group, a carbodiimide group, bifunctional cross-linkers, NHS-maleimido, and combinations thereof. Thus, the invention provides pharmaceutical compositions resulting from the methods, wherein the composition comprises a carrier-drug compound containing a linkage selected from the group consisting of a thiol linkage, an amide linkage, an oxime linkage, a hydrazone linkage, and a thiazolidinone linkage. In another embodiment, the conjugating step is accomplished by cycloaddition reactions.

The invention provides a pharmaceutical carrier comprising a formula I.

B-(L)^{a}-S-(M)^{b}-C I

Wherein:

B is a targeting group that is a non-hormonal vitamin D, analog, or metabolite thereof conjugated at the carbon 3 position to L¹;

S is a scaffold moiety, comprising poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, polylactic acid, a water-soluble polymer, a small carbon chain linker, or an additional therapeutic moiety;

C is an amine-reactive group, a thiol-reactive group, a maleimide group, a thiol group, a disulfide group, an aldehyde group, an NETS-ester group, a 4-nitrophenyl ester, an acylimidazole, a haloacetyl group, an iodoacetyl group, a bromoacetyl groups, a SMCC group, a sulfo SMCC group, a carbodiimide group and bifunctional cross-linkers such as NHS-maleimido or combinations thereof;

(L)^{a} and (M)^{b} are linkers independently selected from —(CH₂)ₙ—, —C(O)NH—, —HNC(O)—, —C(O)O—, —OC(O)—, —O—, —S—S—, —S—, —S(O)—, —S(O)₂— and —NH—;

a is an integer from 0-4; and

b is an integer from 0-4; and

n is an integer from 0-3.

The invention provides a pharmaceutical carrier comprising formula V:

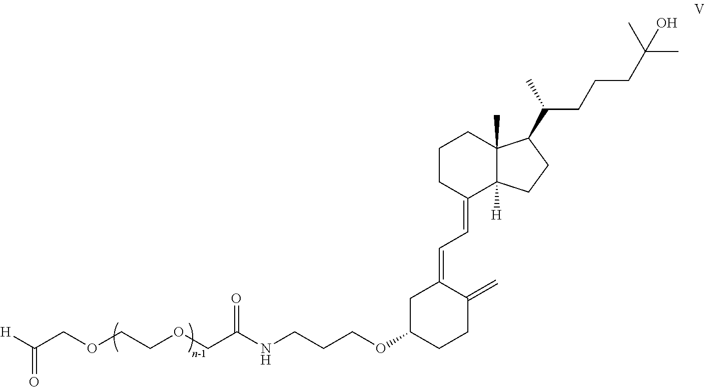

The invention provides a pharmaceutical carrier comprising formula VI:

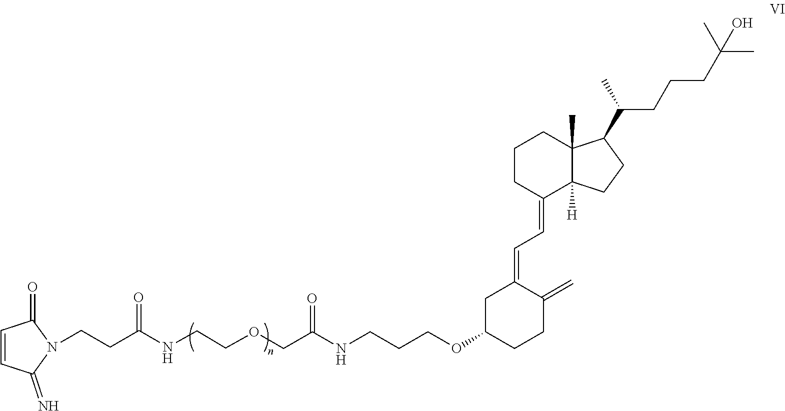

The invention provides a pharmaceutical carrier comprising formula VII:

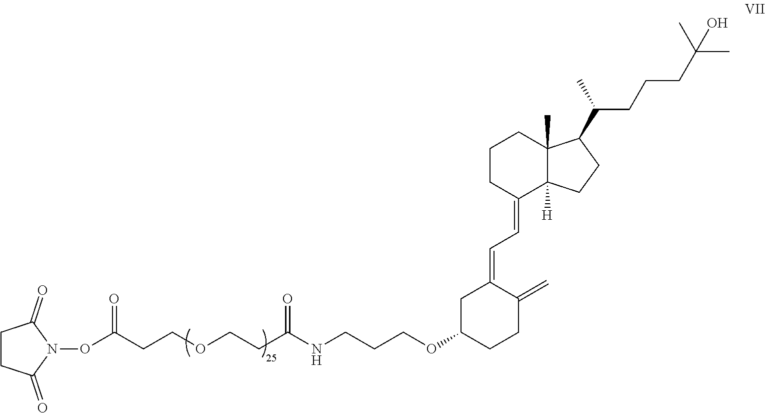

The invention provides a pharmaceutical composition, comprising a therapeutic compound, a stably attached scaffold, a targeting group that is a non-hormonal vitamin D, analog, or metabolite thereof conjugated at the carbon 3 position, wherein after administration to a first test subject, the therapeutic compound has a half life measured by ELISA analysis of blood samples taken at a plurality of time points that is greater than a half life of the therapeutic compound administered to a second test subject without the stably attached scaffold moiety and targeting group as measured by ELISA analysis of blood samples taken at the plurality of time points. In a preferred embodiment, the administration to the first and second subjects is accomplished by subcutaneous injection. In another preferred embodiment, the therapeutic compound stably attached to the scaffold and targeting group retains substantially the same activity as the therapeutic compound not stably attached to the scaffold and targeting group as measured by a functional assay.

In another preferred embodiment of the pharmaceutical composition, a scaffold mass range is selected from the group consisting of 100 Da. to 20,000 Da., 200 Da. to 15,000 Da., 300 Da. to 10,000 Da., 400 Da. to 9,000 Da., 500 Da. to 5,000 Da., 600 Da. to 2,000 Da., 1000 Da. to 200,000 Da., 20,000 Da. to 200,000 Da., 100,000 to 200,000 Da., 5000 Da. to 100,000 Da., 10,000 Da. to 80,000 Da., 20,000 Da. to 60,000 Da., and 20,000 Da. to 40,000 Da. In a more preferred embodiment, the scaffold is approximately the same mass as the therapeutic compound.

The invention provides a carrier-drug conjugate comprising a targeting group that is vitamin D, an analog, or a metabolite thereof that is non-releasably conjugated to a therapeutic compound. In a preferred embodiment, the vitamin D is non-hormonal. In a more preferred embodiment, the non-hormonal vitamin D is not hydroxylated at the carbon 1 position. In a more preferred embodiment, the therapeutic compound is conjugated at the carbon 3 position of the non-hormonal vitamin D targeting group. In a more preferred embodiment, the therapeutic compound retains substantially the same activity as the therapeutic compound not conjugated to the targeting group as measured by a functional assay. In a more preferred embodiment, the targeting group is conjugated to the therapeutic peptide or said therapeutic nucleic acid via a scaffold that is selected from the group consisting of poly(ethylene glycol), polylysine, polyethyleneimine, poly(propyleneglycol), a peptide, serum albumin, thioredoxin, an immunoglobulin, an amino acid, a nucleic acid, a glycan, a modifying group that contains a reactive linker, a water-soluble polymer, a small carbon chain linker, and an additional therapeutic compound. In a more preferred embodiment, the scaffold is approximately the same mass as the therapeutic compound

In order that the invention described herein may be more fully understood, the following examples are set forth. It should be understood that these examples are for illustrative purposes only and are not to be construed as limiting this invention in any manner. In particular, the compositions and methods disclosed herein function with all non-hormonal forms of vitamin D, including homologs, analogs, and metabolites thereof. This includes vitamin D as used in the examples below:

Polypeptides and Their Function

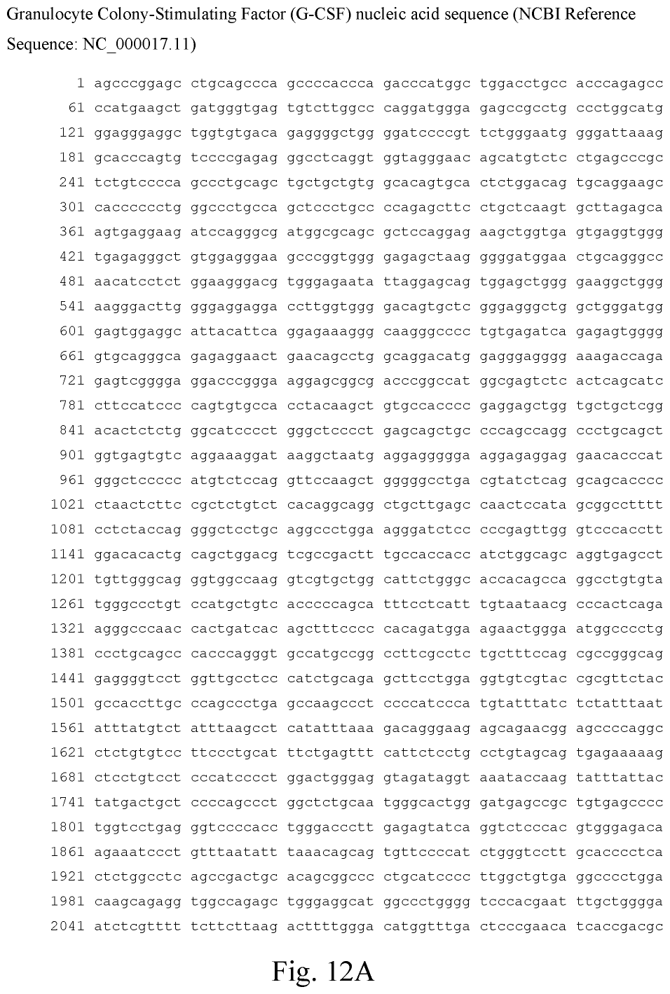

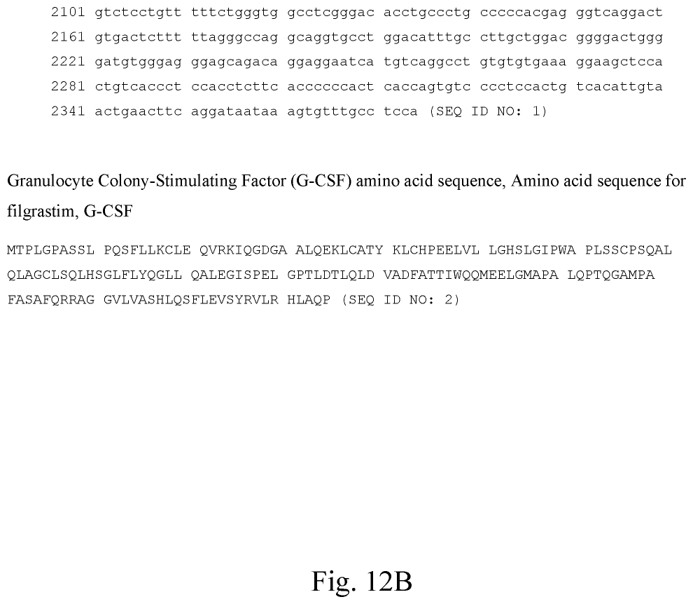

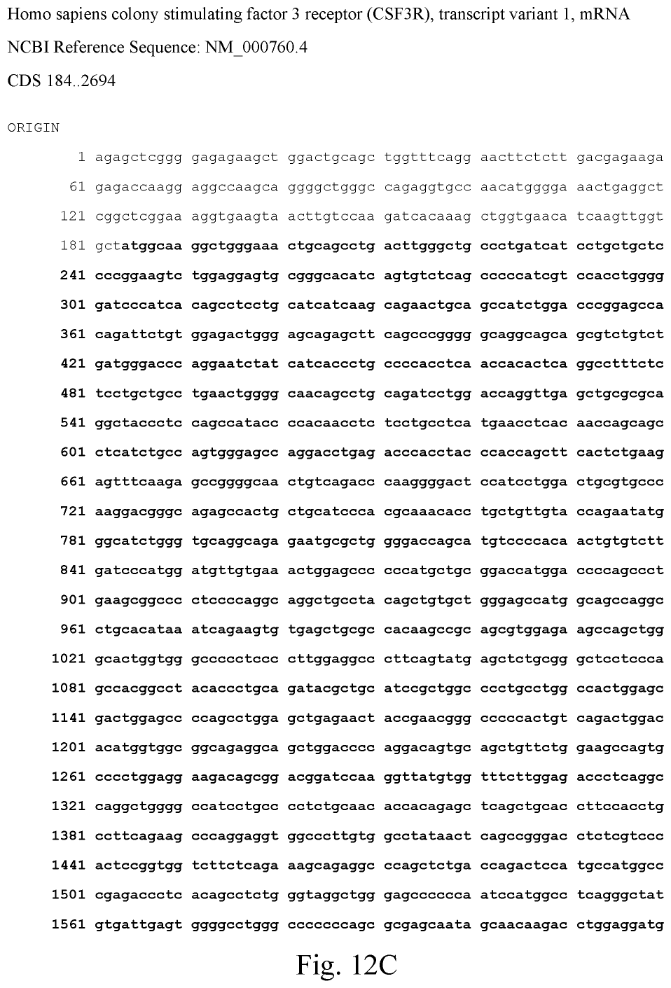

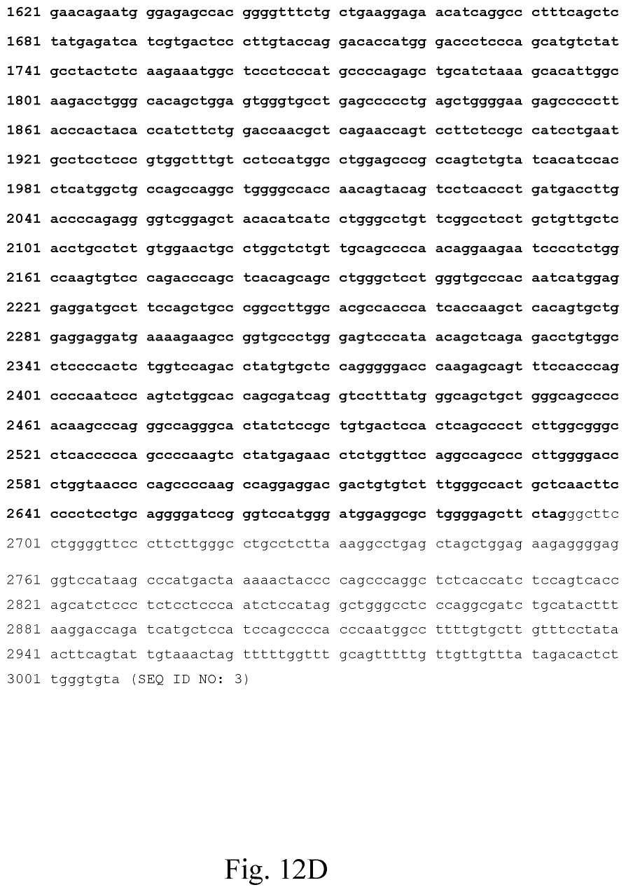

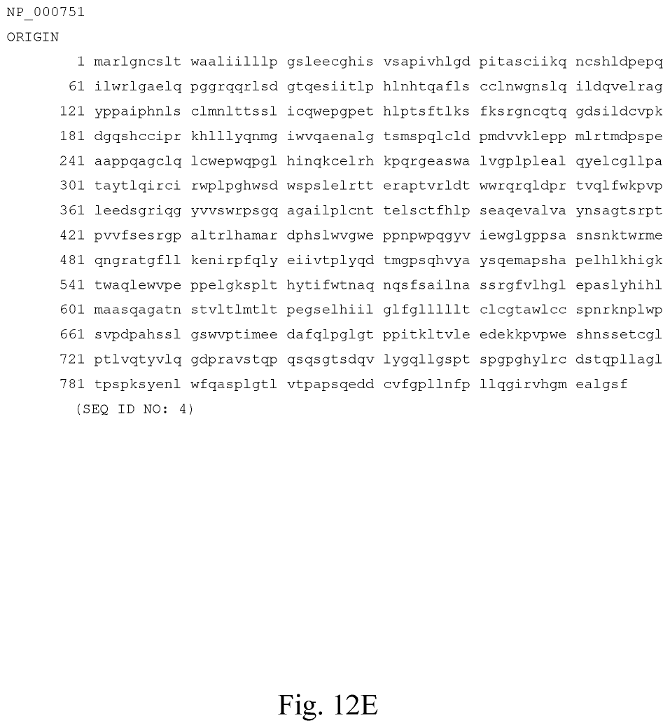

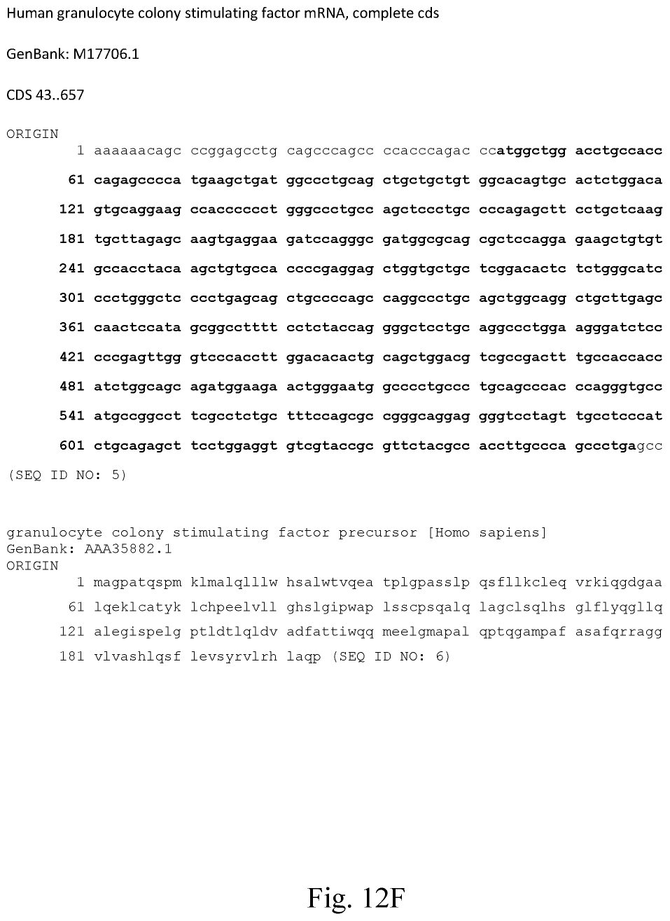

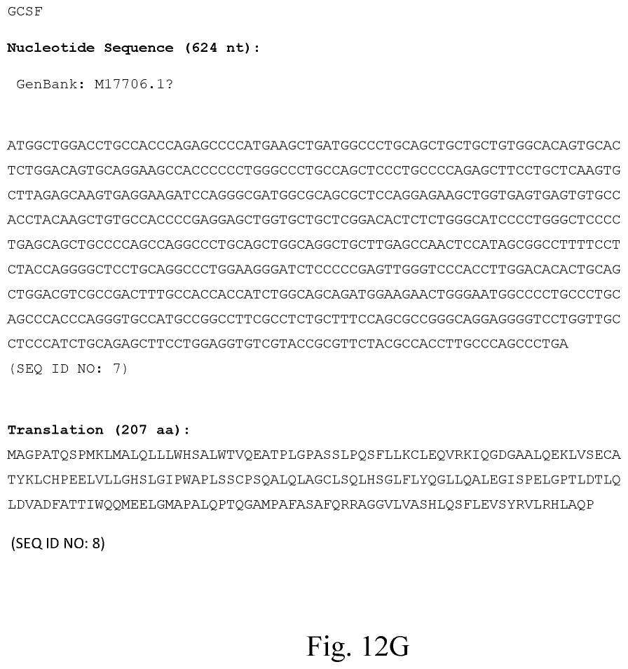

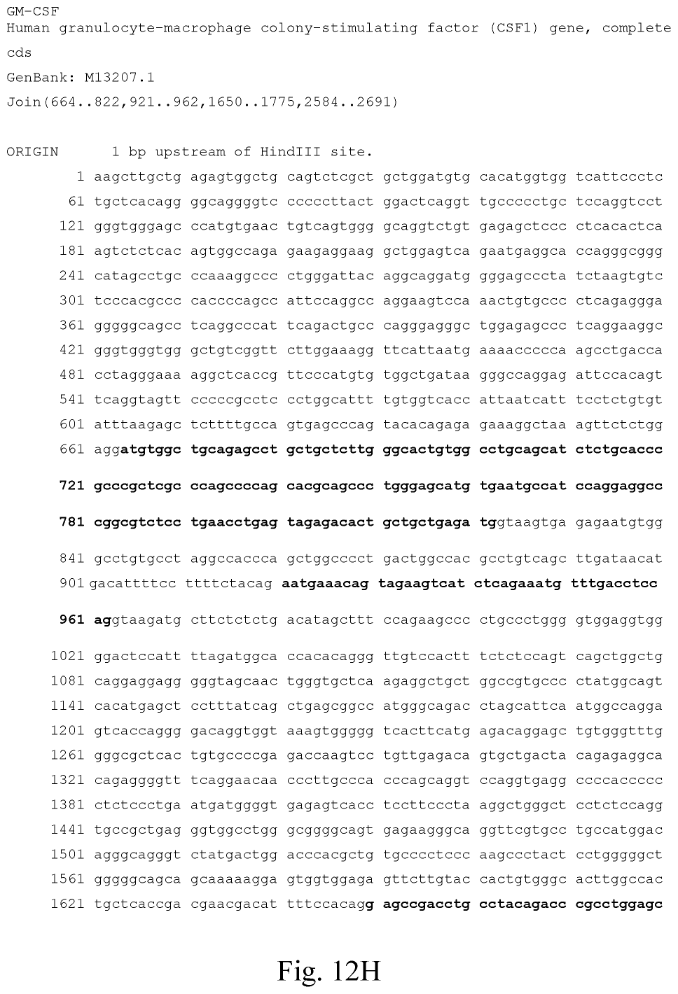

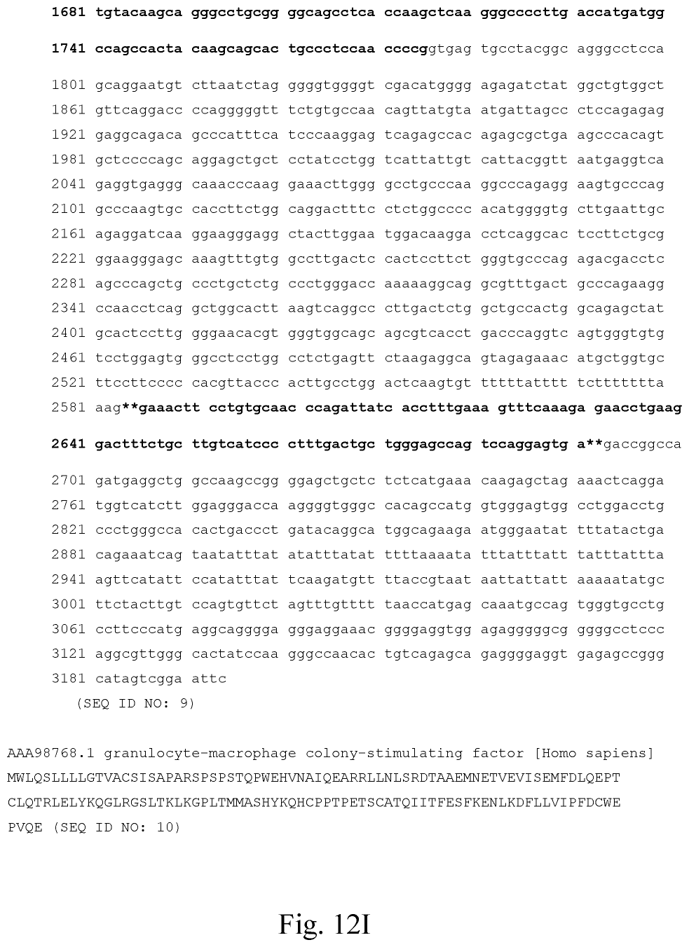

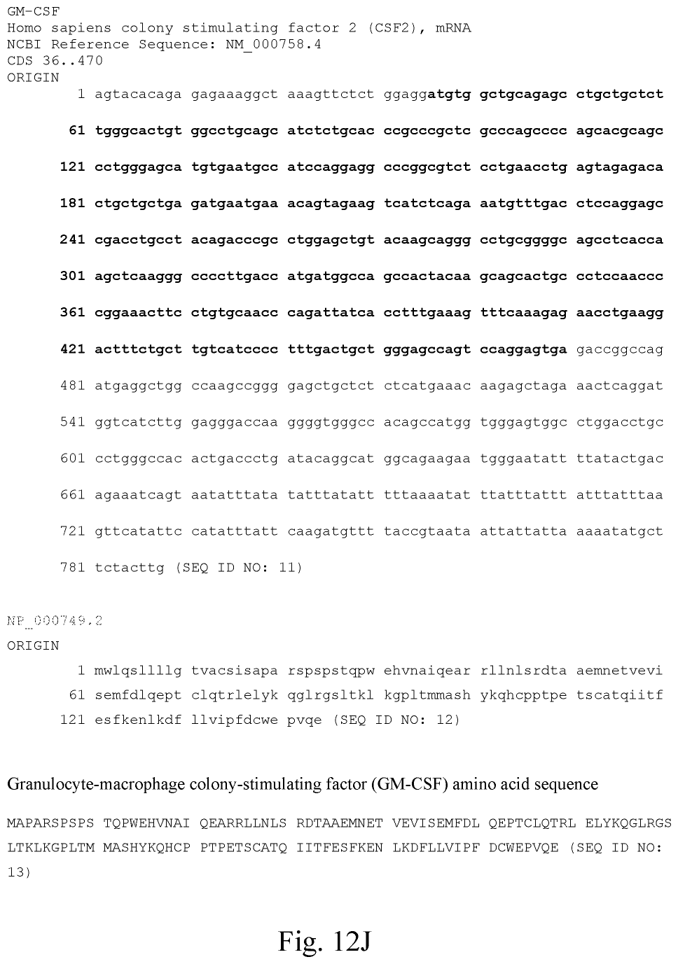

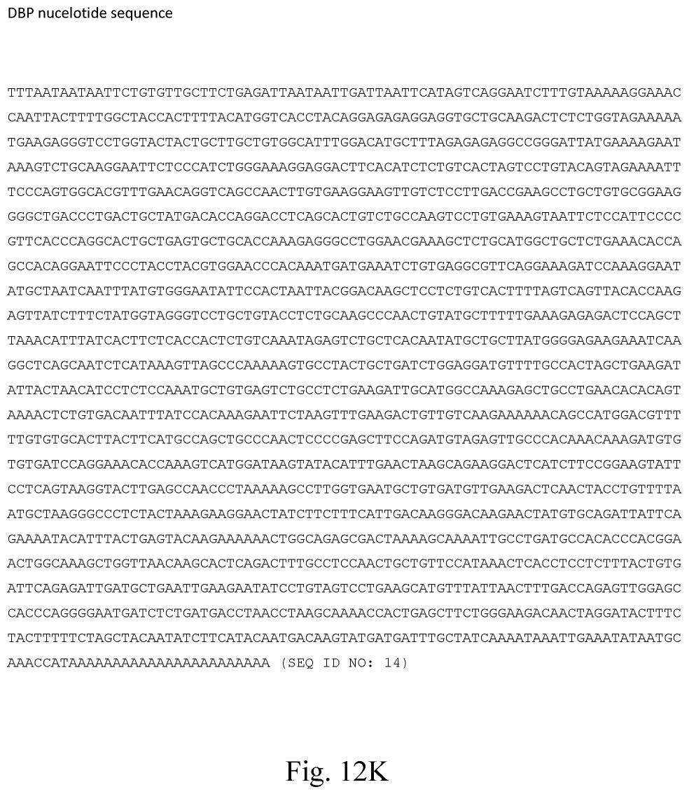

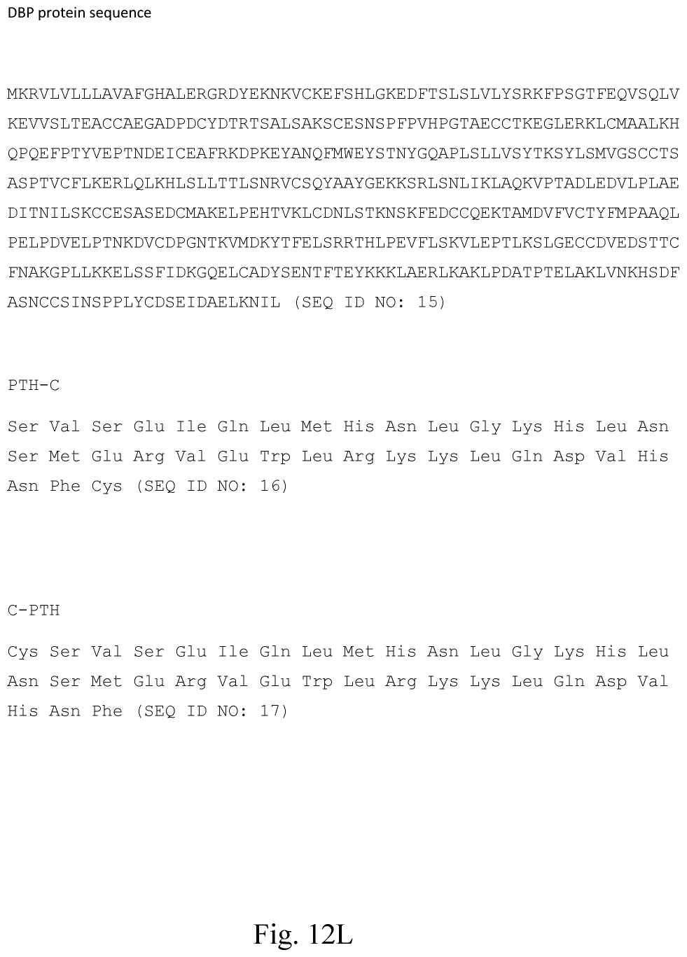

The present invention relates to isolated polypeptide G-CSF or GM-CSF molecules that have been conjugated to carriers, as described herein. The conjugated molecules of present invention include G-CSF or GM-CSF polypeptide molecules that contain the sequence of any one of the amino acid sequences (SEQ ID NO: 2, 4, 6, 8, 10, 12, 13 or combinations thereof). See FIG. 12. The present invention also pertains to conjugated polypeptide molecules include a G-CSF or GM-CSF portion that are encoded by nucleic acid sequences, SEQ ID NO: 1, 3, 5, 7, 9, 11 or combinations thereof). See FIG. 12.

As used herein, the term “polypeptide” encompasses amino acid chains of any length, including full length proteins, wherein the amino acid residues are linked by covalent peptide bonds. Thus, a polypeptide comprising an immunogenic or functional portion of a G-CSF or GM-CSF can consist entirely of the immunogenic portion or can contain additional sequences. The additional sequences can be derived from the native G-CSF or GM-CSF protein or can be heterologous, and such sequences can (but need not) be immunogenic. In general, the polypeptides disclosed herein are prepared in substantially pure form. Preferably, the polypeptides are at least about 80% pure, more preferably at least about 90% pure and most preferably at least about 99% pure.

G-CSF or GM-CSF polypeptides included in the conjugate of the present invention referred to herein as “isolated” are polypeptides that separated away from other proteins and cellular material of their source of origin. Isolated G-CSF or GM-CSF polypeptides include essentially pure protein, proteins produced by chemical synthesis, by combinations of biological and chemical synthesis and by recombinant methods. The G-CSF or GM-CSF proteins included in the conjugate of the present invention have been isolated and characterized as to its physical characteristics using the procedures and can be done using laboratory techniques for protein purification. Such techniques include, for example, salting out, immunoprecipation, column chromatography, high pressure liquid chromatography, and electrophoresis.

The compositions and methods of the conjugate of present invention also encompass G-CSF or GM-CSF variants of the above polypeptides and DNA molecules. A polypeptide “variant,” as used herein, is a polypeptide that differs from the recited polypeptide only in conservative substitutions and/or modifications, such that the diagnostic, therapeutic, and/or functional properties of the polypeptide are retained. A variant of a G-CSF or GM-CSF used in the present invention will therefore be useful in methods described herein. Polypeptide variants preferably exhibit at least about 70%, more preferably at least about 90% and most preferably at least about 95% homology to the identified polypeptides. For polypeptides with immunoreactive properties, variants can, alternatively, be identified by modifying the amino acid sequence of one of the above polypeptides and evaluating the immunoreactivity of the modified polypeptide. Such modified sequences can be prepared and tested using, for example, the representative procedures described herein.

As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

G-CSF or GM-CSF variants can also, or alternatively, contain other modifications, including the deletion or addition of amino acids that have minimal influence on the diagnostic or functional properties, secondary structure and hydropathic nature of the polypeptide. For example, a polypeptide can be conjugated to a signal (or leader) sequence at the N-terminal end of the protein which co-translationally or post-translationally directs transfer of the protein. The polypeptide can also be conjugated to a linker or other sequence for ease of synthesis, purification or identification of the polypeptide (e.g., poly-His), or to enhance binding of the polypeptide to a solid support. For example, a polypeptide can be conjugated to an immunoglobulin Fc region.

The conjugates of present invention also encompass G-CSF or GM-CSF proteins and polypeptides, variants thereof, or those having amino acid sequences analogous to the amino acid sequences of functional G-CSF or GM-CSF polypeptides. Such polypeptides are defined herein as G-CSF or GM-CSF analogs (e.g., homologues), or mutants or derivatives. “Analogous” or “homologous” amino acid sequences refer to amino acid sequences with sufficient identity of any one of the G-CSF or GM-CSF amino acid sequences so as to possess the biological activity of any one of the native G-CSF or GM-CSF polypeptides. For example, an analog polypeptide can be produced with “silent” changes in the amino acid sequence wherein one, or more, amino acid residues differ from the amino acid residues of any one of the G-CSF or GM-CSF protein, yet still possesses the function or biological activity of the G-CSF or GM-CSF. Examples of such differences include additions, deletions or substitutions of residues of the amino acid sequence of G-CSF or GM-CSF. Also encompassed by the conjugate of present invention are analogous polypeptides that exhibit greater, or lesser, biological activity of any one of the G-CSF or GM-CSF proteins of the present invention. Such polypeptides can be made by mutating (e.g., substituting, deleting or adding) one or more amino acid or nucleic acid residues to any of the isolated G-CSF or GM-CSF molecules described herein. Such mutations can be performed using methods described herein and those known in the art. In particular, the present invention relates to homologous polypeptide molecules having at least about 70% (e.g., 75%, 80%, 85%, 90% or 95%) identity or similarity with SEQ ID NO: 2, 4, 6, 8, 10, 12, 14, 16, 18, 20, 22, or combination thereof. Percent “identity” refers to the amount of identical nucleotides or amino acids between two nucleotides or amino acid sequences, respectfully. As used herein, “percent similarity” refers to the amount of similar or conservative amino acids between two amino acid sequences.

The polypeptides of the conjugate of present invention include full length sequences, partial sequences, functional fragments and homologues, that allow for or assist in stimulating an immunogenic specific or protective immune response to G-CSF or GM-CSF. For G-CSF “functional” as used herein, refers to the ability to stimulate the bone marrow to produce granulocytes and stem cells and release them into the bloodstream in a patient, such as a human, and/or in a biological sample. For GM-CSF, “functional” refers to it's ability to functions as a cytokine, to function as a white blood cell growth factor and to stimulate stem cells to produce granulocytes (neutrophils, eosinophils, and basophils) and monocytes. Functional portions of the polypeptide described herein can be prepared and identified using the techniques described herein. Other techniques, such as those summarized in Paul, Fundamental Immunology, 3d ed., Raven Press, 1993, pp. 243-247 and references cited therein, can be used. Such techniques include screening polypeptide portions of the native protein. A functional portion of polypeptide can generate at least about 20%, and preferably about 100%, of the activity induced by the full length protein described herein.

Homologous polypeptides can be determined using methods known to those of skill in the art. Initial homology searches can be performed at NCBI against the GenBank, EMBL and SwissProt databases using, for example, the BLAST network service. Altschuler, S. F., et al., J. Mol. Biol., 215:403 (1990), Altschuler, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 73:237-244 (1988) e.g., using default parameters).

Additionally, the individual isolated polypeptides of the conjugate of present invention are biologically active or functional. The present invention includes fragments of these isolated amino acid sequences yet possess the function or biological activity of the sequence. For example, polypeptide fragments comprising deletion mutants of the G-CSF or GM-CSF proteins can be designed and expressed by well-known laboratory methods. Fragments, homologues, or analogous polypeptides can be evaluated for biological activity, as described herein.

The conjugate of present invention also encompasses biologically active derivatives or analogs of the above described G-CSF or GM-CSF polypeptides, referred to herein as peptide mimetics. Mimetics can be designed and produced by techniques known to those of skill in the art. (see e.g., U.S. Pat. Nos. 4,612,132; 5,643,873 and 5,654,276). These mimetics can be based, for example, on a specific G-CSF or GM-CSF amino acid sequence and maintain the relative position in space of the corresponding amino acid sequence. These peptide mimetics possess biological activity similar to the biological activity of the corresponding peptide compound, but possess a “biological advantage” over the corresponding G-CSF or GM-CSF amino acid sequence with respect to one, or more, of the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Modifications of peptides to produce peptide mimetics are described in U.S. Pat. Nos. 5,643,873 and 5,654,276. Other forms of the G-CSF or GM-CSF polypeptides, encompassed by the present invention, include those which are “functionally equivalent.” This term, as used herein, refers to any nucleic acid sequence and its encoded amino acid, which mimics the biological activity of the G-CSF or GM-CSF polypeptides and/or functional domains thereof.

Nucleic Acid Sequences, Plasmids, Vectors and Host Cells

The conjugate of the present invention, in one embodiment, includes isolated G-CSF or GM-CSF nucleic acid molecule having a sequence of SEQ ID NO: 1, 3, 5, 7, 9, 11 or combinations thereof. See FIG. 12. As used herein, the terms “DNA molecule” or “nucleic acid molecule” include both sense and anti-sense strands, cDNA, genomic DNA, recombinant DNA, RNA, and wholly or partially synthesized nucleic acid molecules. A nucleotide “variant” is a sequence that differs from the recited nucleotide sequence in having one or more nucleotide deletions, substitutions or additions. Such modifications can be readily introduced using standard mutagenesis techniques, such as oligonucleotide-directed site-specific mutagenesis as taught, for example, by Adelman et al. (DNA 2:183, 1983). Nucleotide variants can be naturally occurring allelic variants, or non-naturally occurring variants. Variant nucleotide sequences preferably exhibit at least about 70%, more preferably at least about 80% and most preferably at least about 90% homology to the recited sequence. Such variant nucleotide sequences will generally hybridize to the recited nucleotide sequence under stringent conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

The present invention also encompasses isolated nucleic acid sequences that encode G-CSF or GM-CSF polypeptides, and in particular, those which encode a polypeptide molecule having an amino acid sequence of SEQ ID NO: 2, 4, 6, 8, 10, 12, 13 or combinations thereof. These G-CSF or GM-CSF nucleic acid sequences encode polypeptides that stimulate or supplement G-CSF or GM-CSF function as described herein.

As used herein, an “isolated” gene or nucleotide sequence which is not flanked by nucleotide sequences which normally (e.g., in nature) flank the gene or nucleotide sequence (e.g., as in genomic sequences) and/or has been completely or partially purified from other transcribed sequences (e.g., as in a cDNA or RNA library). Thus, an isolated gene or nucleotide sequence can include a gene or nucleotide sequence which is synthesized chemically or by recombinant means. Nucleic acid constructs contained in a vector are included in the definition of “isolated” as used herein. Also, isolated nucleotide sequences include recombinant nucleic acid molecules and heterologous host cells, as well as partially or substantially or purified nucleic acid molecules in solution. In vivo and in vitro RNA transcripts of the present invention are also encompassed by “isolated” nucleotide sequences. Such isolated nucleotide sequences are useful for the manufacture of the encoded G-CSF or GM-CSF polypeptide, as probes for isolating homologues sequences (e.g., from other mammalian species or other organisms), for gene mapping (e.g., by in situ hybridization), or for detecting the presence (e.g., by Southern blot analysis) or expression (e.g., by Northern blot analysis) of related genes in cells or tissue.

The G-CSF or GM-CSF nucleic acid sequences of the present invention include homologues nucleic acid sequences. “Analogous” or “homologous” nucleic acid sequences refer to nucleic acid sequences with sufficient identity of any one of the G-CSF or GM-CSF nucleic acid sequences, such that once encoded into polypeptides, they possess the biological activity of any one of the G-CSF or GM-CSF polypeptides described herein. For example, an analogous nucleic acid molecule can be produced with “silent” changes in the sequence wherein one, or more, nucleotides differ from the nucleotides of any one of the G-CSF or GM-CSF polypeptides described herein, yet, once encoded into a polypeptide, still possesses its function or biological activity. Examples of such differences include additions, deletions or substitutions. Also encompassed by the present invention are nucleic acid sequences that encode analogous polypeptides that exhibit greater, or lesser, biological activity of the G-CSF or GM-CSF proteins of the present invention. In particular, the present invention is directed to nucleic acid molecules having at least about 70% (e.g., 75%, 80%, 85%, 90% or 95%) identity with SEQ ID NO: 1, 3, 5, 7, 9, 11, or combinations thereof.

The nucleic acid molecules included in the conjugate the present invention, including the full length sequences, the partial sequences, functional fragments and homologues, once encoded into polypeptides, elicit a specific G-CSF or GM-CSF response, or has the function of the G-CSF or GM-CSF polypeptide, as further described herein. The homologous nucleic acid sequences can be determined using methods known to those of skill in the art, and by methods described herein including those described for determining homologous polypeptide sequences. Functional portions of the polypeptide can then be sequenced using techniques such as Edman chemistry. See Edman and Berg, Eur. J. Biochem. 80:116-132, 1967.

Also encompassed by the conjugate of present invention are nucleic acid sequences, DNA or RNA, which are substantially complementary to the DNA sequences encoding the G-CSF or GM-CSF polypeptides of the present invention, and which specifically hybridize with their DNA sequences under conditions of stringency known to those of skill in the art. As defined herein, substantially complementary means that the nucleic acid need not reflect the exact sequence of the G-CSF or GM-CSF sequences, but must be sufficiently similar in sequence to permit hybridization with G-CSF or GM-CSF nucleic acid sequence under high stringency conditions. For example, non-complementary bases can be interspersed in a nucleotide sequence, or the sequences can be longer or shorter than the G-CSF or GM-CSF nucleic acid sequence, provided that the sequence has a sufficient number of bases complementary to the G-CSF or GM-CSF sequence to allow hybridization therewith. Conditions for stringency are described in e.g., Ausubel, F. M., et al., Current Protocols in Molecular Biology, (Current Protocol, 1994), and Brown, et al., Nature, 366:575 (1993); and further defined in conjunction with certain assays.

Also encompassed by the conjugate present invention are nucleic acid sequences, genomic DNA, cDNA, RNA or a combination thereof, which are substantially complementary to the DNA sequences of the present invention and which specifically hybridize with the G-CSF or GM-CSF nucleic acid sequences under conditions of sufficient stringency (e.g., high stringency) to identify DNA sequences with substantial nucleic acid identity.

The present invention also includes portions and other variants of G-CSF or GM-CSF that are generated by synthetic or recombinant means. Synthetic polypeptides having fewer than about 100 amino acids, and generally fewer than about 50 amino acids, can be generated using techniques well known to those of ordinary skill in the art. For example, such polypeptides can be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc., Foster City, Calif., and can be operated according to the manufacturer's instructions. Variants of a native protein can generally be prepared using standard mutagenesis techniques, such as oligonucleotide-directed site-specific mutagenesis. Sections of the DNA sequence can also be removed using standard techniques to permit preparation of truncated polypeptides.

In another embodiment, the conjugate of present invention includes nucleic acid molecules (e.g., probes or primers) that hybridize to the G-CSF or GM-CSF sequences, SEQ ID NO: 1, 3, 5, 7, 9, 11 or combinations thereof under high or moderate stringency conditions. In one aspect, the present invention includes molecules that are or hybridize to at least about 20 contiguous nucleotides or longer in length (e.g., 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000). Such molecules hybridize to one of the G-CSF or GM-CSF nucleic acid sequences under high stringency conditions. The present invention includes such molecules and those that encode a polypeptide that has the functions or biological activity described herein.

Typically the nucleic acid probe comprises a nucleic acid sequence (e.g. SEQ ID NO: 1, 3, 5, 7, 9, 11, or combinations thereof) and is of sufficient length and complementarity to specifically hybridize to a nucleic acid sequence that encodes a G-CSF or GM-CSF polypeptide. For example, a nucleic acid probe can be at least about 5%, 10%, 20%, 30%, 40%, 50%, 60%, 70%, 80% or 90% the length of the G-CSF or GM-CSF nucleic acid sequence. The requirements of sufficient length and complementarity can be easily determined by one of skill in the art. Suitable hybridization conditions (e.g., high stringency conditions) are also described herein. Additionally, the present invention encompasses fragments of the polypeptides of the present invention or nucleic acid sequences that encodes a polypeptide wherein the polypeptide has the biologically activity of the G-CSF or GM-CSF polypeptides recited herein.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences can be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tm) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na3-citrate . . . 2H₂O (88 g/liter), pH to 7.0 with 1 M HCl), 1% SDS (sodium dodecyl sulfate), 0.1-2 mg/ml denatured calf thymus DNA at 65° C., (2) 1×SSC, 50% formamide, 1% SDS, 0.1-2 mg/ml denatured calf thymus DNA at 42° C., (3) 1% bovine serum albumin (fraction V), 1 mM Na₂ . . . EDTA, 0.5 M NaHPO₄ (pH 7.2) (1 M NaHPO₄=134 g Na₂HPO₄ . . . 7H₂O, 4 ml 85% H₃PO₄ per liter), 7% SDS, 0.1-2 mg/ml denatured calf thymus DNA at 65° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 Dg/ml denatured calf thymus DNA at 65° C., or (6) 5×SSC, 5×Denhardt's solution, 50% formamide, 1% SDS, 100 □g/ml denatured calf thymus DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na3-citrate . . . 2H₂O (88 g/liter), pH to 7.0 with 1 M HCl), 1% SDS (sodium dodecyl sulfate), 0.1-2 mg/ml denatured calf thymus DNA at 65° C., (2) 4×SSC, 50% formamide, 1% SDS, 0.1-2 mg/ml denatured calf thymus DNA at 42° C., (3) 1% bovine serum albumin (fraction V), 1 mM Na₂ . . . EDTA, 0.5 M NaHPO₄ (pH 7.2) (1 M NaHPO₄=134 g Na2HPO₄ . . . 7H₂O, 4 ml 85% H₃PO₄ per liter), 7% SDS, 0.1-2 mg/ml denatured calf thymus DNA at 65° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 Dg/ml denatured calf thymus DNA at 65° C., or (6) 5×SSC, 5×Denhardt's solution, 50% formamide, 1% SDS, 100 □g/ml denatured calf thymus DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na+), and “L” is the length of the hybrid in base pairs.

Low stringency conditions can employ hybridization at either (1) 4×SSC, (10×SSC=3 M NaCl, 0.3 M Na3-citrate . . . 2H₂O (88 g/liter), pH to 7.0 with 1 M HCl), 1% SDS (sodium dodecyl sulfate), 0.1-2 mg/ml denatured calf thymus DNA at 50° C., (2) 6×SSC, 50% formamide, 1% SDS, 0.1-2 mg/ml denatured calf thymus DNA at 40° C., (3) 1% bovine serum albumin (fraction V), 1 mM Na₂ . . . EDTA, 0.5 M NaHPO₄ (pH 7.2) (1 M NaHPO₄=134 g Na₂HPO₄ . . . 7H₂O, 4 ml 85% H₃PO₄ per liter), 7% SDS, 0.1-2 mg/ml denatured calf thymus DNA at 50° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 40° C., (5) 5×SSC, 5×Denhardt's solution, 1% SDS, 100 Dg/ml denatured calf thymus DNA at 50° C., or (6) 5×SSC, 5×Denhardt's solution, 50% formamide, 1% SDS, 100 □g/ml denatured calf thymus DNA at 40° C., with low stringency washes of either 2×SSC, 0.1% SDS at 50° C., or (2) 0.5% bovine serum albumin (fraction V), 1 mM Na₂EDTA, 40 mM NaIPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na.+), and “L” is the length of the hybrid in base pairs.

The G-CSF or GM-CSF nucleic acid sequences used in the conjugate of the present invention, or a fragment thereof, can also be used to isolate additional homologs. For example, a cDNA or genomic DNA library from the appropriate organism can be screened with labeled G-CSF or GM-CSF nucleic acid sequence to identify homologous genes as described in e.g., Ausebel, et al., Eds., Current Protocols In Molecular Biology, John Wiley & Sons, New York (1997).

Functional polypeptides can be produced recombinantly using a DNA sequence that encodes the protein, which has been inserted into an expression vector and expressed in an appropriate host cell. DNA sequences encoding G-CSF or GM-CSF can, for example, be identified by screening an appropriate G-CSF or GM-CSF genomic or cDNA expression library with sera obtained from patients having G-CSF or GM-CSF. Such screens can generally be performed using techniques well known to those of ordinary skill in the art, such as those described in Sambrook et al., Molecular Cloning: A Laboratory Manual, Cold Spring Harbor Laboratories, Cold Spring Harbor, N.Y., 2001. Degenerate oligonucleotide sequences for use in such a screen can be designed and synthesized, and the screen can be performed. Polymerase chain reaction (PCR) can also be employed, using the above oligonucleotides in methods well known in the art, to isolate a nucleic acid probe from a cDNA or genomic library. The library screen can then be performed using the isolated probe. The present method can optionally include a labeled G-CSF or GM-CSF probe.

Alternatively, genomic or cDNA libraries can be screened directly using peripheral blood mononuclear cells (PBMCs) or T cell lines or clones. In general, PBMCs and/or T cells for use in such screens can be prepared as described below. Direct library screens can generally be performed by assaying pools of expressed recombinant proteins for the ability to induce proliferation and/or interferon-□ production in T cells.

Recombinant polypeptides for the conjugate of the present invention containing portions and/or variants of a native protein can be readily prepared from a DNA sequence encoding the polypeptide using a variety of techniques well known to those of ordinary skill in the art. For example, supernatants from suitable host/vector systems which secrete recombinant protein into culture media can be first concentrated using a commercially available filter. Following concentration, the concentrate can be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art can be employed to express recombinant polypeptides of this invention. Expression can be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. The DNA sequences expressed in this manner can encode naturally occurring polypeptide, portions of naturally occurring polypeptide, or other variants thereof.

Uses of plasmids, vectors or viruses containing the conjugate of the present invention including G-CSF or GM-CSF proteins or fragments include one or more of the following; (1) generation of hybridization probes for detection and measuring level of G-CSF or GM-CSF or isolation of G-CSF or GM-CSF homologs; (2) generation of G-CSF or GM-CSF mRNA or protein in vitro or in vivo; and (3) generation of transgenic non-human animals or recombinant host cells.

In one embodiment, the present invention encompasses host cells transformed with the plasmids, vectors or viruses described above. Nucleic acid molecules can be inserted into a construct which can, optionally, replicate and/or integrate into a recombinant host cell, by known methods. The host cell can be a eukaryote or prokaryote and includes, for example, yeast (such as Pichia pastorius or Saccharomyces cerevisiae), bacteria (such as E. coli, L. infantum, or Bacillus subtilis), animal cells or tissue, insect Sf9 cells (such as baculoviruses infected SF9 cells) or mammalian cells (somatic or embryonic cells, Human Embryonic Kidney (HEK) cells, Chinese hamster ovary cells, HeLa cells, human 293 cells and monkey COS-7 cells). Host cells suitable in the present invention also include a mammalian cell, a bacterial cell, a yeast cell, an insect cell, and a plant cell.

The nucleic acid molecule can be incorporated or inserted into the host cell by known methods. Examples of suitable methods of transfecting or transforming cells include calcium phosphate precipitation, electroporation, microinjection, infection, lipofection and direct uptake. “Transformation” or “transfection” as used herein refers to the acquisition of new or altered genetic features by incorporation of additional nucleic acids, e.g., DNA. “Expression” of the genetic information of a host cell is a term of art which refers to the directed transcription of DNA to generate RNA which is translated into a polypeptide. Methods for preparing such recombinant host cells and incorporating nucleic acids are described in more detail in Ausubel, F. M., et al., Current Protocols in Molecular Biology, (John Wiley & Sons, 2004) and Sambrook et al., “Molecular Cloning: A Laboratory Manual,” (2001), for example.

The host cell is then maintained under suitable conditions for expression and recovery of the G-CSF or GM-CSF polypeptide of the present invention. Generally, the cells are maintained in a suitable buffer and/or growth medium or nutrient source for growth of the cells and expression of the gene product(s). The growth media are not critical to the invention, are generally known in the art and include sources of carbon, nitrogen and sulfur. Examples include Luria broth, Superbroth, Dulbecco's Modified Eagles Media (DMEM), RPMI-1640, M199 and Grace's insect media. The growth media can contain a buffer, the selection of which is not critical to the invention. The pH of the buffered Media can be selected and is generally one tolerated by or optimal for growth for the host cell.

The host cell is maintained under a suitable temperature and atmosphere. Alternatively, the host cell is aerobic and the host cell is maintained under atmospheric conditions or other suitable conditions for growth. The temperature should also be selected so that the host cell tolerates the process and can be for example, between about 13-40 degree Celsius.

### EXEMPLIFICATION

Examples other than Vitamin D/G-CSF conjugates are included to assist in understanding the manufacturing technology employed and the pharmacokinetic impact of the invention. The exemplifications apply as well to conjugates of Vitamin D/GM-CSF.

All exemplary work was performed by Extend Biosciences Inc. of Newton Mass.

### Example 1: Preparation of an Exemplary Thiol-Reactive Carrier Composed of Vitamin D₃-PEG with a Maleimide Reactive Group

The maleimide on the carrier in this example was used to conjugate to a free cysteine on a protein or peptide. It is contemplated that the size of the PEG in the scaffolds of the invention are from 0.1 kDa to 100 kDa. Thus, a 2 kDa PEG was selected as a scaffold for this example. The starting materials used in this example were purchased from commercial sources: Toronto Research Chemicals for the Vitamin D analog (compound 1, Toronto Research Chemicals Catalog No. B691610) and from Creative Pegworks for the 2 kDa mPEG-maleimide (compound 4, Creative PEGworks Catalog No. PHB-940).

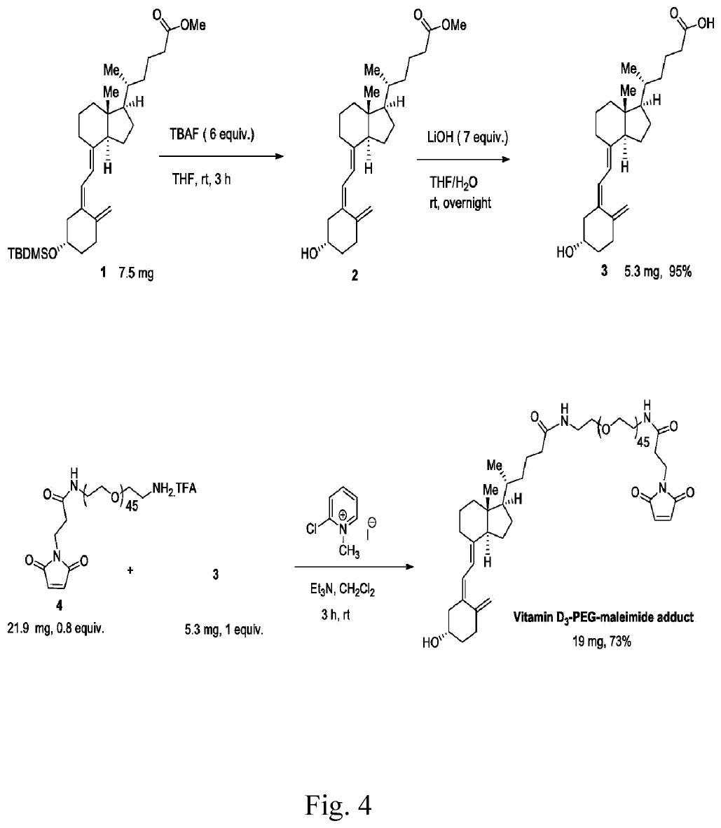

According to FIG. 4, (R)-methyl5-((1R,3aS,7aR,E)-4-((Z)-2-((S)-5-((tert-butyldimethylsilyl)oxy)-2-methylenecy-clohexylidene)ethylidene)-7a-methyloctahydro-1H-inden-1-yl)hexanoate (compound 1, 7.5 mg, 0.0145 mmol, 1 equiv., purchased from Toronto Research Chemicals) was dissolved in anhydrous tetrahydrofuran (0.4 mL) and flushed with nitrogen. Tetrabutylammonium fluoride (22.7 mg, 0.087 mmol, 6 equiv.) was added and the reaction was stirred at room temperature for 3 hours with monitoring by thin layer chromatography (TLC, silica gel, 30% ethyl acetate in hexanes, UV detection, phosphomolybdic acid stain). To the resulting mixture containing compound 2 was added lithium hydroxide monohydrate (4.2 mg, 0.1015 mmol, 7 equiv.), tetrahydrofuran (0.3 mL) and water (0.15 mL). The reaction was flushed with nitrogen and stirred at room temperature for 18 hours. Evaluation by TLC and mass spectroscopy (MS) indicated complete reaction with the presence of expected compound 3. The reaction mixture was diluted with ether (2×15 mL) and washed with 10% aqueous citric acid (30 mL), water (30 mL) and brine (30 mL). The organic layer was dried over anhydrous sodium sulfate and concentrated while maintaining the temperature below 20° C. The sample was further dried under a stream of nitrogen giving ((R)-5-((1R,3aS,7aR,E)-4-((Z)-2-((S)-5-hydroxy-2-methylenecyclohexylidene)ethyl-idene)-7a-methyloctahydro-1H-inden-1-yl)hexanoic acid) (compound 3, 5.3 mg, 95% yield) as a colorless gum. Rf 0.2 (silica gel, 40% EtOAc in hexanes). NMR analysis revealed the presence of about 1.14% of THE and about 0.14% of ether.

Compound 3 (5.3 mg, 0.0137 mmol, 1 equiv.), compound 4 (MAL-PEG-amine TFA salt, 21.9 mg, 0.0109 mmol, 0.8 equiv., purchased from Creative Pegworks) and 2-chloro-1-methylpyridinium iodide (8.7 mg, 0.0342 mmol, 2.5 equiv.) were dissolved in anhydrous dichloromethane (0.5 mL). Triethylamine (7.6 μL, 0.0548 mmol, 4 equiv.) was added and the reaction mixture was stirred for 3 hours at room temperature under nitrogen. The reaction was then diluted with dichloromethane (30 mL) and washed with 10% aqueous citric acid (40 mL), saturated aqueous sodium bicarbonate (30 mL) and brine (30 mL). The organic layer was dried over anhydrous sodium sulfate, filtered and concentrated while maintaining the temperature below 20° C. The sample was further dried under a stream of nitrogen to afford the target compound as a brown gum. Rf 0.6 (silica gel, 20% methanol in dichloromethane). TLC analysis (ninhydrin stain) of the isolated product indicated the absence of compound 4. 1H NMR analysis of the isolated material confirmed its identity and purity. The NMR analysis did not show an appreciable amount of methylene chloride or other solvents.

### Example 2 Preparation of an Exemplary Amine-Reactive Carrier Composed of Vitamin D₃-PEG with a NHS-Reactive Group

NHS-reactive groups on carriers were generated for conjugation to amine groups on proteins. A 2 kDa PEG was selected as a scaffold for this example. The starting materials used in this example were purchased from Toronto Research Chemicals for the Vitamin D analog (compound 1) and from Creative Pegworks for the 2 kDa mPEG-amino acid (compound 5).

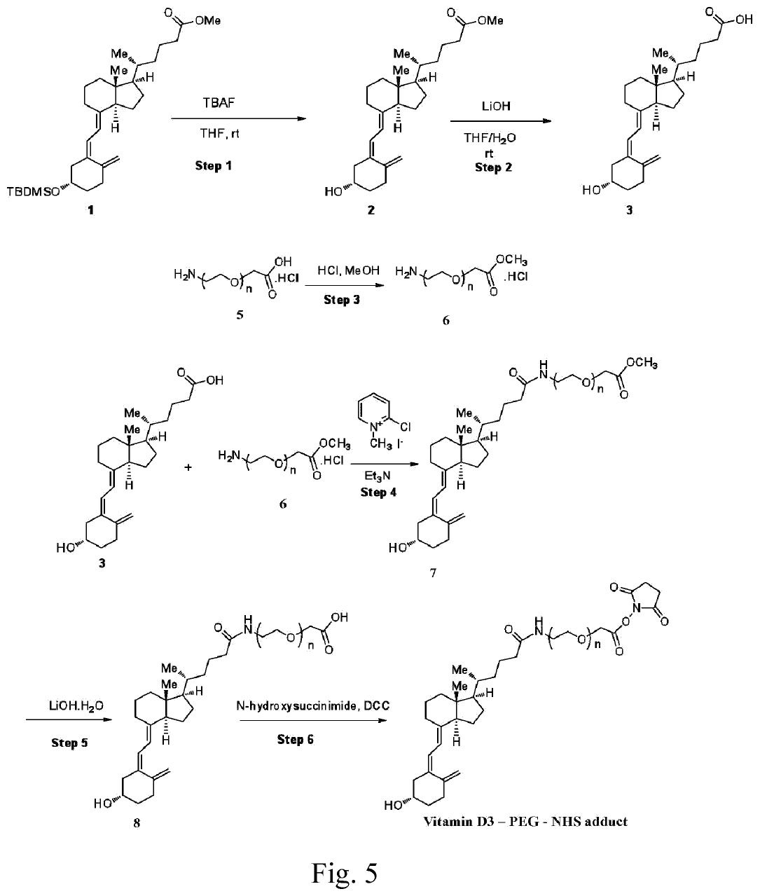

According to FIG. 5 (steps 1 and 2): (R)-Methyl-5-((1R,3aS,7aR,E)-4-((Z)-2-((S)-5-((tert-butyldimethylsilyl)oxy)-2-methylenecy-clohexylidene)ethylidene)-7a-methyloctahydro-1H-inden-1-yl)hexanoate (compound 1, 8.2 mg, 0.0159 mmol, 1 equiv.) was dissolved in anhydrous tetrahydrofuran (THF, 0.4 mL) and the mixture was flushed with nitrogen. Tetrabutylammonium fluoride solution (25 mg, 0.096 mmol, 6 equiv.) was added and the reaction mixture was stirred at room temperature for 3 hr with monitoring by thin layer chromatography (TLC, silica gel, 30% ethyl acetate in hexanes, UV detection, phosphomolybdic acid stain). To the resulting mixture containing compound 2, lithium hydroxide monohydrate (4.6 mg, 0.109 mmol, 7 equiv.), THE (0.3 mL), and water (0.16 mL) were added. The reaction mixture was flushed with nitrogen and stirred at room temperature for 18 hr. Evaluation by TLC and mass spectroscopy (MS) indicated complete reaction with the presence of the expected compound 3. The reaction mixture was diluted with ether (10 mL) and washed with 10% aqueous citric acid (10 mL), water (10 mL) and brine (10 mL). The organic layer was dried over anhydrous sodium sulfate and concentrated while maintaining the temperature below 20° C. The sample was further dried under a stream of nitrogen giving ((R)-5-((1R,3aS,7aR,E)-4-((Z)-2-((S)-5-hydroxy-2-methylenecyclohexylidene)ethyl-indene)-7a-methyloctahydro-1H-inden-1-yl)hexanoic acid (compound 3, 6.1 mg, 99% yield) as a colorless gum. Rf 0.2 (silica gel, 40% ethyl acetate (EtOAc) in hexanes). Nuclear magnetic resonance spectroscopy (NMR) analysis revealed the presence of ^{˜}7% of ether.

According to FIG. 5 (step 3): to a solution of PEG-amino acid 4 (18.5 mg, 0.0092 mmol, purchased from Creative Pegworks) in anhydrous methanol, HCl in dioxane (4 M, 1.5 mL) was added, and the reaction mixture was heated at 70° C. in a sealed tube for 20 hr. The reaction was monitored by TLC (ninhydrin stain), and upon completion of the reaction, it was concentrated on a rotavap. The residue was co-evaporated with dichloromethane (3×5 mL) and ether (3×5 mL) to a pale yellow foam, which was suspended in ether (5 mL). The liquid was decanted and the solid obtained was dried to isolate the desired product 5 (14 mg, 75%) as a pale yellow solid. Rf 0.2 (silica gel, 20% methanol (MeOH)/DCM/0.2% NH4OH). NMR analysis did not show an appreciable amount of methylene chloride or ether.

According to FIG. 5 (step 4): compound 3 (3.4 mg, 0.009 mmol, 1 equiv.), compound 6 (methyl ester PEG-amine HCl salt, 14 mg, 0.007 mmol, 0.8 equiv.) and 2-chloro-1-methylpyridinium iodide (5.6 mg, 0.022 mmol, 2.5 equiv.) were dissolved in anhydrous dichloromethane (0.6 mL). Triethylamine (5 μL, 0.0356 mmol, 4 equiv.) was added and the reaction mixture was stirred for 3 hr at room temperature under nitrogen. The reaction was incomplete at this time, therefore an additional amount of compound 3 (1.7 mg, 0.0045 mmol) was added and the reaction was continued further 3 hr, then diluted with dichloromethane (10 mL) and washed with 10% aqueous citric acid (10 mL), saturated aqueous sodium bicarbonate (10 mL) and brine (10 mL). The organic layer was dried over anhydrous sodium sulfate, filtered and concentrated while maintaining the temperature below 20° C. The sample was purified by silica gel (2 g) flash chromatography. The column was first eluted with ethyl acetate to remove unreacted compound 3 and then with 1-10% MeOH/dichloromethane (20 mL each). Fractions containing pure product were combined together and evaporated on a rotavap, while maintaining the temperature below 20° C. The sample was dried under a stream of nitrogen to afford compound 7 as a brown gum (10 mg, 60%). Rf 0.3 (silica gel, 5% MeOH in dichloromethane). TLC analysis (ninhydrin stain) of the isolated product indicated the absence of compound 6. 1H NMR analysis of the isolated material confirmed its identity and purity. The NMR analysis revealed the presence of 1.1% of methylene chloride.

According to FIG. 5 (step 5): compound 7 (10 mg, 0.0042 mmol) was dissolved in a mixture of THE (0.2 mL) and a drop of methanol. To this solution was added lithium hydroxide monohydrate solution (0.9 mg, 0.021 mmol, 5 equiv. in 0.1 mL of water). The reaction mixture was flushed with nitrogen and stirred at room temperature for 18 hr. Evaluation by TLC indicated complete reaction with the presence of compound 8. The reaction mixture was diluted with dichloromethane (10 mL) and washed with 10% aqueous citric acid (10 mL) and brine (10 mL). The organic layer was dried over anhydrous sodium sulfate and concentrated while maintaining the temperature below 20° C. The sample was further dried under a stream of nitrogen giving the desired Vitamin-D₃-PEG-acid (compound 8, 7 mg, 71% yield) as a brown gum. Rf 0.2 (silica gel, 10% MeOH/dichloromethane). NMR analysis revealed the presence of ^{˜}2% of dichloromethane.

Stock solutions were prepared: 34 g of N-hydroxysuccinimide in 1 mL of anhydrous dimethylformamide (DMF) and 61 mg of dicyclohexylcarbodiimide (DCC) in 1 mL of anhydrous dichloromethane.

According to FIG. 5 (step 6): to a solution of compound 8 (7 mg, 0.003 mmol, 1 equiv.) in dichloromethane (0.3 mL) was added a solution of N-hydroxysuccinimide in DMF (10 μL, 0.34 mg, 0.003 mmol) followed by a solution of DCC in dichloromethane (10 μL, 0.61 mg, 0.003 mmol) and the reaction mixture was flushed with nitrogen and stirred for 20 hr. Since the reaction was incomplete as indicated by TLC, additional amounts of N-hydroxysuccinimide in DMF (25 μL, 0.85 mg, 0.0075 mmol) and DCC in dichloromethane (25 μL, 1.53 mg, 0.0075 mmol) were added and the reaction was continued for another 20 hr. Chloroform (10 mL) was added to the reaction mixture, and it was washed with water (10 mL). The organic phase was dried over sodium sulfate and the removal of solvent provided the desired target compound Vitamin D₃-PEG-NHS (7.0 mg, crude). ¹H NMR and TLC (R_{f}: 0.3, 10% MeOH/chloroform) of this material indicated the presence of desired material.

### Example 3:Preparation Exemplary Carriers for Coupling Therapeutic Compounds to Non-Hormonal Vitamin D at the C₂₅ Position

Exemplary carriers were prepared containing vitamin D and 2 kDa PEG scaffolds. One exemplary carrier was thiol-reactive and comprised vitamin D-PEG with a maleimide reactive group at the C₂₅ position (herein referred to as Vitamin D-(25)-PEG₂ₖ-maleimide or VitD-(25)-PEG₂ₖ-maleimide).

Another exemplary carrier was amine-reactive and comprised vitamin D-PEG with an NETS-reactive group. These reagents were prepared as described in WO2013172967 (Soliman et al.), incorporated herein by reference in its entirety.

### Example 4:Preparation of an Exemplary Amino-Terminal Reactive Carrier for Coupling Therapeutic

Compounds to Non-Hormonal Vitamin D at the C3 Position

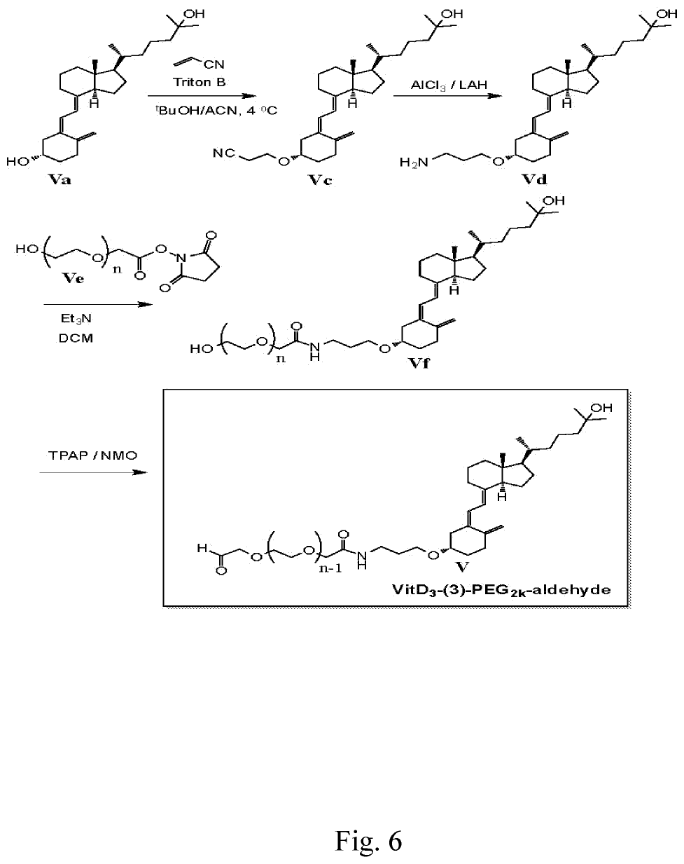

An exemplary amino-terminal reactive carrier was prepared containing an aldehyde reactive group connected to the C3 position of vitamin D and a 2 kDa PEG scaffold (herein referred to as Vitamin D-(3)-PEG₂ₖ-aldehyde or VitD-(3)-PEG₂ₖ-maleimide). The aldehyde on the carrier in this example was used to conjugate to a free amino-terminus on the proteins and peptides disclosed in the examples below. The synthesis is outlined in FIG. 6.

Briefly, (S,Z)-3-((E)-2-((1R,3aS,7aR)-1-((R)-6-hydroxy-6-methylheptan-2-yl)-7a-methylhexahydro-1H-inden-4(2H)-ylidene)ethylidene)-4-methylenecyclohexanol (compound Va, 20 mg, 0.049 mmol, 1 equiv., purchased from Toronto Research Chemicals, catalog number C₁₂₅₇₀₀, also known as calcifediol and 25-hydroxyvitamin D) was dissolved in a mixture of anhydrous tert-butanol and acetonitrile (10:1, 1 mL), cooled to 4° C. Acrylonitrile (26.6 mg, 0.5 mmol, 10 equiv.) was added to it followed by Triton B, 40% aqueous solution, 10 μL). The mixture was stirred at 4° C. for 2.5 h. The reaction was quenched with cold 2% HCl (10 mL), the aqueous phase was extracted with ether (2×10 mL), dried (MgSO₄) and evaporated to obtain the crude product. This material was purified by flash chromatography (TLC, silica gel, 50% ethyl acetate in hexanes) with 5-20% EtOAc/hexanes as eluent to isolate the desired product, 3-(((S,Z)-3-((E)-2-((1R,3aS,7aR)-1-((R)-6-hydroxy-6-methylheptan-2-yl)-7a-methylhexahydro-1H-inden-4(2H)-ylidene)ethylidene)-4-methylenecyclohexyl)oxy)propanenitrile, compound V (15 mg, 68%) as a white solid (R_{f}0.2 silica gel, 40% EtOAc in hexanes). NMR analysis did not show any appreciable amount of solvents. To a solution of aluminum chloride (66 mg, 0.495 mmol) in anhydrous ether (2 mL) at 0° C. under argon was added a solution of lithium aluminum hydride (1M in ether, 19 mg, 0.5 mL, 0.5 mmol) dropwise. The mixture was stirred for 5 min., a solution of compound Vc (15 mg, 0.033 mmol) in ether (3 mL) was added to it dropwise, the reaction mixture was stirred at 0° C. for 5 min and then at room temperature for 1 h. The reaction was monitored by MS and TLC (silica gel, 10% MeOH/CHCl₃/0.1% NH40H). Ethyl acetate (1 mL) and water (1 mL) were added to the reaction mixture followed by 5% NaOH (5 mL). The organic phase was separated, and the aqueous phase was extracted with ethyl acetate (5 mL) and ether (5 mL). The combined organic phases were washed with brine (5 mL), dried (Na₂SO₄) and evaporated on a rotavap to afford the desired amine, (R)-6-((1R,3aS,7aR,E)-4-((Z)-2-((S)-5-(3-aminopropoxy)-2-methylenecyclohexylidene)ethylidene)-7a-methyloctahydro-1H-inden-1-yl)-2-methylheptan-2-ol, compound Vd (12.5 mg, 82%) as a pale yellow oil. R_{f}0.2 (silica gel, 20% MeOH/DCM/0.2% NH40H). The NMR analysis revealed the presence ^{˜}8% of ethyl acetate. Compound Vd (12.5 mg, 0.0273 mmol, 1 equiv.), compound Ve (hydroxyl PEG NHS ester, MW 2000 with n≅45 where n is the number of repeating CH₂CH₂O units, Jenkem Technology US #A-5076, 43 mg, 0.0216 mmol, 0.8 equiv.) were dissolved in anhydrous dichloromethane (0.1 mL). Triethylamine (12 mg, 16 μl mmol, 4 equiv.) was added and the reaction mixture was stirred for 20 h at room temperature under nitrogen. The sample was dried under a stream of nitrogen to afford the crude compound Vf, which was purified by flash chromatography using 5-10% MeOH/dichloromethane as eluent to isolate the desired product Vf as a white foam (30 mg, 38%). R_{f}0.4 (silica gel, 10% methanol in dichloromethane). ¹H NMR analysis of the isolated material confirmed its identity and purity.

To a solution of compound Vf (30 mg, 0.0123 mmol, 1 equiv.), tetrapropylammonium perruthenate (1.0 mg, 0.00284, 0.23 equiv.) and N-methylmorpholine-N-Oxide (4.3 mg, 0.0369 mmol, 3 equiv.) in 2 mL of dry dichloromethane was added powdered 4 A° molecular sieves (500 mg) and the reaction mixture was flushed with N₂. The reaction flask was covered with aluminum foil to avoid light and it was stirred at room temperature for 36 h. Since the R_{f} of both starting material and product is same on TLC (silicagel, 10% MeOH/dichloromethane), formation of the product was confirmed by examining the ¹H NMR of an aliquot.

The reaction mixture was filtered through the pad of Celite in a pipette with dichloromethane (15 mL) and N₂ pressure. The combined organics were concentrated under a flow of N₂ and dried on high vacuum for 2 h to get 35 mg (100%) of the crude product TLC (R_{f}: 0.3, 10% MeOH/dichloromethane, staining with PMA). A second run of reaction under the exactly same conditions yielded another 35 mg of the product. ¹H NMR of the product from both batches is same and hence combined to get 70 mg of compound V, VitD-(3)-PEG₂ₖ-aldehyde.

### Example 5:Preparation of an Exemplary Thiol-Reactive Carrier for Coupling Therapeutic Compounds to Non-Hormonal Vitamin D at the C3 Position

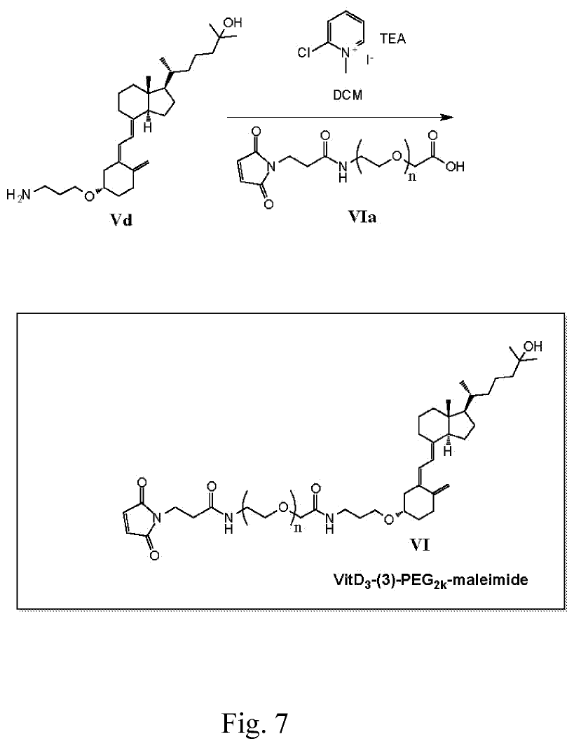

An exemplary thiol-reactive carrier comprising vitamin D with a maleimide reactive group connected to the C3 position of vitamin D (VitD-(3)-PEG₂ₖ-maleimide) was prepared. The maleimide on the carrier in this example was used to conjugate to a free thiol on the protein and peptide in the examples below. The synthesis is outlined in FIG. 7.

Briefly, compound Vd (23 mg, 0.05 mmol, 1 equiv.) prepared as in Example 2, compound VIa (Creative Pegworks cat. #PHB-956, MAL-PEG-COOH, 2 k with n˜45 where n is the number of repeating CH₂CH₂O units, 79 mg, 0.0395 mmol, 0.8 equiv.) and 2-chloro-1-methylpyridinium iodide (32 mg, 0.125 mmol, 2.5 equiv.) were dissolved in anhydrous dichloromethane (1 mL). Triethylamine (20.4 mg, 28 μl, 0.2 mmol, 4 equiv.) was added and the reaction mixture was stirred for 4 h at room temperature under nitrogen. The reaction mixture was diluted with dichloromethane (20 mL), washed with 5% aqueous citric acid (20 mL), saturated aqueous sodium bicarbonate (20 mL), and brine (20 mL). The organic layer was dried over anhydrous sodium sulfate, filtered and concentrated at 30° C. The sample was purified by silica gel (10 g) flash chromatography. The column was eluted with 1-10% MeOH/dichloromethane. Fractions containing pure product were combined together and evaporated on a rotavap, while maintaining the temperature at 30° C. The sample was dried under a stream of nitrogen to afford compound VI, VitD-(3)-PEG₂ₖ-maleimide as a brown gum (58 mg, 48%) (R_{f}0.25, silica gel, 10% methanol in dichloromethane). ¹H NMR analysis of the isolated material confirmed its identity and purity.

### Example 6: Preparation of an Exemplary Amine-Reactive Carrier for Coupling Therapeutic

Compounds to Non-Hormonal Vitamin D at the C3 Position

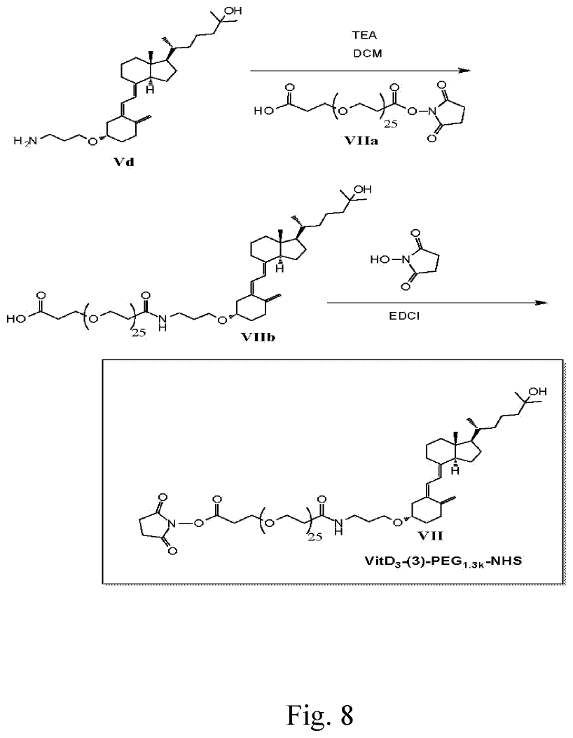

An exemplary amine-reactive carrier comprising vitamin D with an NHS reactive group connected to the C3 position of vitamin D (Herein referred to as Vitamin D-(3)-PEG_{1.3k}-NHS or VitD-(3)-PEG_{1.3k}-NHS) was prepared. The NHS on the carrier in this example was used to conjugate to a free thiol on the protein and peptide in the examples below. The synthesis is outlined in FIG. 8.

Briefly, compound Vd (20 mg, 0.044 mmol, 1 equiv.) and compound VIIa (Quanta Biodesign cat. #10140, with n=25 where n is the number of repeating CH₂CH₂O units, 44 mg, 0.0346 mmol, 0.8 equiv.) were dissolved in anhydrous dichloromethane (1 mL). Triethylamine (22.0 mg, 31 μl, 0.22 mmol, 5 equiv.) was added and the reaction mixture was stirred for 24 h at room temperature under nitrogen. The reaction mixture was diluted with dichloromethane (20 mL), washed with 5% aqueous citric acid (20 mL), and brine (20 mL). The organic layer was dried over anhydrous sodium sulfate, filtered and concentrated while maintaining the temperature at 30° C. The sample was purified by silica gel (10 g) flash chromatography. The column was eluted with 1-10% MeOH/dichloromethane. Fractions containing pure product were combined together and evaporated on a rotavap, while maintaining the temperature below 30° C. The sample was dried under a stream of nitrogen to afford compound VIIb as a brown gum (33 mg, 56%) (R_{f}0.20, silica gel, 10% methanol in dichloromethane). ¹H NMR analysis of the isolated material confirmed its identity.

Compound VIIb (31 mg, 0.018 mmol, 1 equiv.), N-hydroxysuccinimide (6.3 mg, 0.055 mmol, 3 equiv.), and EDCI (8.6 mg, 0.045 mmol, 2.5 eq.) were dissolved in anhydrous THE (2 mL). Triethylamine (7.4 mg, 10 μL, 0.073 mmol, 4 equiv.) was added and the reaction mixture was stirred for 24 h at room temperature under nitrogen. The reaction mixture was diluted with dichloromethane (20 mL) and washed with 5% aqueous citric acid (20 mL), and brine (20 mL).

The organic layer was dried over anhydrous sodium sulfate, filtered and concentrated while maintaining the temperature at 30° C. The sample was dried under a stream of nitrogen to afford compound VII, VitD-(3)-PEG₂ₖ-NHS, as a brown gum (38.6 mg, >100%) (R_{f}0.25, silica gel, 10% methanol in dichloromethane). ¹H NMR analysis of the isolated material confirmed its identity and purity.

### Example 7: Comparison of G-CSF, PEG-G-CSF, and VitDG-CSF with Regards to PK, Efficacy of Progenitor Cell Mobilization, and Induction of Biomarkers

Summary:

Using Extend Bioscience's D-VITylation technology, vitamin D will be attached to G-CSF by a short PEG linker. The resultant VitD-G-CSF will be compared to unmodified G-CSF (Neupogen, filgrastim) and PEG-G-CSF (Neulasta, pegfilgrastim). A single dose will be subcutaneously administered to rats and blood collected for sampling over ten days. Whole blood will be analyzed for progenitor cell markers (CD34, VEGFR2, CD133) by flow cytometry. Serum will be analyzed for drug pharmacokinetics and levels of biomarkers using ELISA (HGF, ANG-1, MMP-9, HGF, PDGF-AA, PAI-1).

Preparation of VitD-G-CSF:

G-CSF will be modified with Extend Biosciences compound #0-9851, which is VitD-PEG36-NHS with a molecular weight of 2,356 g/mol. The NHS group (Nhydroxysuccinimide) of 0-9851 is reactive with amines on G-CSF, of which there are five: the N-terminus, and positions K16, K23, K34, and K40. Test reactions will be performed with different ratios of 0-9851 to G-CSF such that on average, just over one molecule of 0-9851 is added to each G-CSF, purified by ion exchange, and characterized by MALDI-TOF mass spectrometry and SDS-PAGE (gel electrophoresis).

### Results Synthesis of GCSF-PEG₂₄-VitD

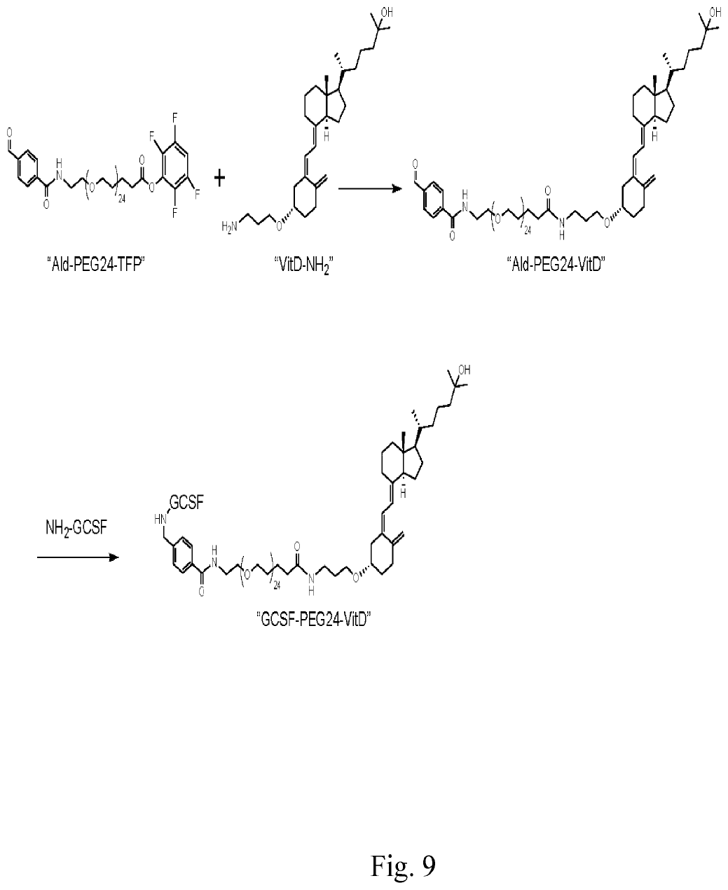

Vitamin D was attached to the N-terminus of G-CSF via a PEG linker as shown in FIG. 9.

“VitD-NH2” was prepared according to Soliman U.S. Pat. No. 9,585,934 B2. “ald-PEG₂₄-TFP” was obtained from Quanta BioDesign (4-formyl-benzamido-dPEG₂₄-TFP ester, cat #10082). VitD-NH2 (10 mg) was dissolved in DMSO (1 ml) and mixed with ald-PEG₂₄-TFP (25 mg, 0.8 equivalents) in 1.25 ml DMSO. The reaction was allowed to proceed for 30 minutes at room temperature. The reaction was purified by reverse phase HPLC on a Waters XSelect CSH Phenyl-hexyl OBD Prep column using the following gradient: 80% solvent A/20% solvent B for 10 minutes, increase to 50% solvent B over 5 minutes, and then to 100% solvent B over 30 minutes. Solvent A is 0.1 M TEAA pH=7 and solvent B is 80% acetonitrile/20% 0.1 M TEAA pH=7. Fractions containing ald-PEG₂₄-VitD were lyophilized to dryness and dissolved in DMSO.

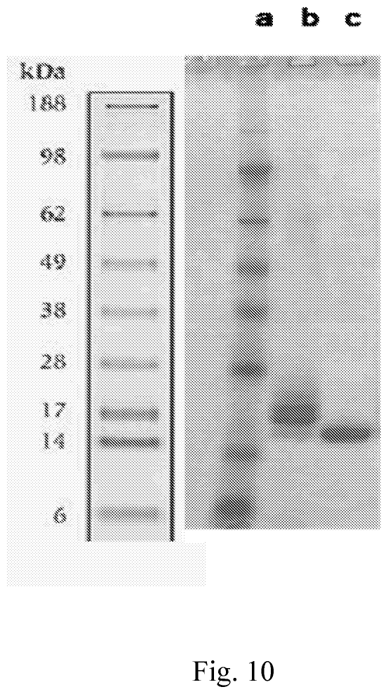

G-CSF (0.15 mg) in 0.075 ml of 50 mM sodium acetate pH=5 buffer was mixed with 46.4 micrograms (3.4 equivalents) of ald-PEG₂₄-VitD in 2 microliters of DMSO. To this mixture was added 3 microliters of a 50 mM solution of NaCNBH₃ and 2.25 microliters of a 10% Tween 80 aqueous solution. The reaction was allowed to proceed overnight at room temperature. The reaction was analyzed by SDS-PAGE using a Novex™ 16% tricine gel (Thermo Fisher Scientific) as shown in FIG. 10. The presence of protein bands in the reaction with a higher molecular weight than G-CSF indicate the reaction was successful with one or more vitamin D-PEG linkers added to G-CSF.

Assay of In Vitro Activity:

G-CSF, PEG-G-CSF, and VitD-G-CSF will be assayed for induction of cell proliferation in a cell line (NSF-60) expressing the G-CSF receptor (see Crobu et al. BMC Pharm and Tox 2014, 15:7).

Alternatively, during the development of pegfilgrastim, attachment of a 20 kDa PEG non-selectively to amines on G-CSF caused a reduction in activity. [CITE] Therefore, a different chemistry was used to attach the PEG specifically to the Nterminus. It is expected that, due to the much smaller size of the modification proposed here (2 vs. 20 kDa), will not reduce in vitro activity by non-selective modification to amines. However, if a loss in activity is observed, a similar chemistry to selectively modify the N-terminus (albeit with a decreased yield) should be adopted.

Measuring Pharmacokinetics in Rats:

The study design is outlined in Table 3, and the blood collection schedule is given in Table 4. Group 1 receives vehicle only and serves as the control. An n=6 animals per group may be chosen as the minimum while still having a reasonable chance to observe statistically significant changes in biomarker and progenitor cell counts. The dose, 0.1 mg/kg (G-CSF weight only to make each dose mole equivalent), is suggested by the literature. [CITE] There, the typical dose for G-CSF was between 10 and 300 μg/kg daily for three to five days and one example of a single dose of 300 μg/kg. Typical doses for PEG-G-CSF ranged from between 50-500 μg/kg. The selected 0.1 mg/kg (100 μg/kg) dose falls within both ranges and is a reasonable amount to synthesize. (Note: Because the 20 kDa PEG constitutes approximately half of the weight of PEG-GCSF, a 0.1 mg/kg protein weight only dose corresponds to a 0.2 mg/kg absolute weight dose).

TABLE 3
Group assignments and dosing for subcutaneous administration

| Group | Compound | # animals/group | Dose* | Material |
|---|---|---|---|---|
| 1 | Vehicle | 6 | — | |
| 2 | G-CSF | 6 | 0.1 mg/kg | 0.4 mg |
| 3 | PEG-G-CSF | 6 | 0.1 mg/kg | 0.8 mg |
| 4 | VitD-G-CSF | 6 | 0.1 mg/kg | 0.4 mg |

*Weight of G-CSF only, not PEG or VitD.

TABLE 4
Blood collection schedule

| | | | | | | | | | | |
|---|---|---|---|---|---|---|---|---|---|---|
| Time (d): | 0 | | | | 1 | 2 | 3 | 4 | 7 | 10 |
| Time (h): | 0 | 2 | 4 | 8 | 24 | 48 | 72 | 96 | 168 | 240 |
| 0.25 ml for | X | | | | X | X | X | X | X | X |
| flow cyt. | | | | | | | | | | |
| 0.25 ml to | X | X | X | X | X | X | X | X | X | X |
| serum | | | | | | | | | | |

Note: Total volume of blood collected per rat=4.25 ml. Typical blood volume limits are 4.5 ml over 14 days for 300 g rats. Might have to use larger rats, or remove some collection points.

Biomarker Analysis:

Serum may be frozen pending analysis. The expected volume of serum from 250 μl of blood is approximately 125 μl. Table 5 lists the ELISA kits that will be used for the analysis as well as the specified sensitivity and required sample volume (for duplicate analysis). In some cases, in order to preserve serum, the suggested sample volume may be reduced since the lowered sensitivity will still be satisfactory. ELISA kits for plasmin, VEGFC, and FGFb with the required sensitivity that did not require large volumes of sample could not be identified.

TABLE 5
ELISA kits used for PK and biomarker analysis of serum samples

| | | Manuf specifications | | Suggested specs | | | |
|---|---|---|---|---|---|---|---|
| | | Sample | | Sample | | | |
| | | Vol | Sensitivity | Vol | Sensitivity | # | |
| Target | Manufacturer/Cat# | (μl)** | (pg/ml) | (μl)** | (pg/ml) | plates | Cost ($) |
| G-CSF | R&D #SCS50 | 20 | 39-2,500 | 20 | 39-2,500 | 6 | $2,491 |
| PAI-1 | RayBio #ELMPAII-1 | 10 | 80-20,000 | 10 | 80-20,000 | 5 | $2,140 |
| PDGF-AA | RayBio #ELRPDGFAA-1 | 100 | 90-6,000 | 20 | 450-30,000 | 5 | $1,809 |
| HGF | R&D #MHG00 | 20 | 62-4,000 | 20 | 62-4,000 | 5 | $2,595 |
| Total | R&D #RMP900 | 20 | 200-10,000 | 20 | 200-10,000 | 5 | $2,595 |
| MMP-9* | | | | | | | |
| Angp-1 | RayBio#ELRangiopoeitin-1 | 100 | 400-100,000 | 20 | 2,000-500,000 | 5 | $1,805 |
| | | | | 110 | | | $13,435 |

*Total MMP-9 = pro-, active, and TIMP-complexed.
**Sample volume for two duplicate samples.

Analysis of Progenitor Cell Population:

Whole blood (0.25 ml) will be returned to Extend Biosciences for analysis of CD34+, VEGFR2+, and CD133+ cells via flow cytometry. Briefly, cells will be washed and stained with a cocktail of antibodies against CD34, VEGFR2, and CD133, each conjugated with a different fluorophore. Red blood cells will be lysed and the cells fixed. A target of one million cells will be analyzed. Viability analysis will be performed. Based on a concentration of 5 million leukocytes per ml of blood, this will require cells from 0.2 ml of blood.

### Example 8: Synthesis of VitD-PEG-GM-CSF and Measurement of the In Vitro Biological Activity

### Aim:

Using Extend Bioscience's D-VITylation technology, vitamin D will be attached to GM-CSF by a short PEG linker with the expectation that this will prolong the lifetime in the bloodstream without compromising biological activity. VitD-PEG-GM-CSF will be compared to GM-CSF for the ability to activate the endogenous receptor, CSF2RB/CSF2RA.

Preparation of VitD-PEG-GM-CSF:

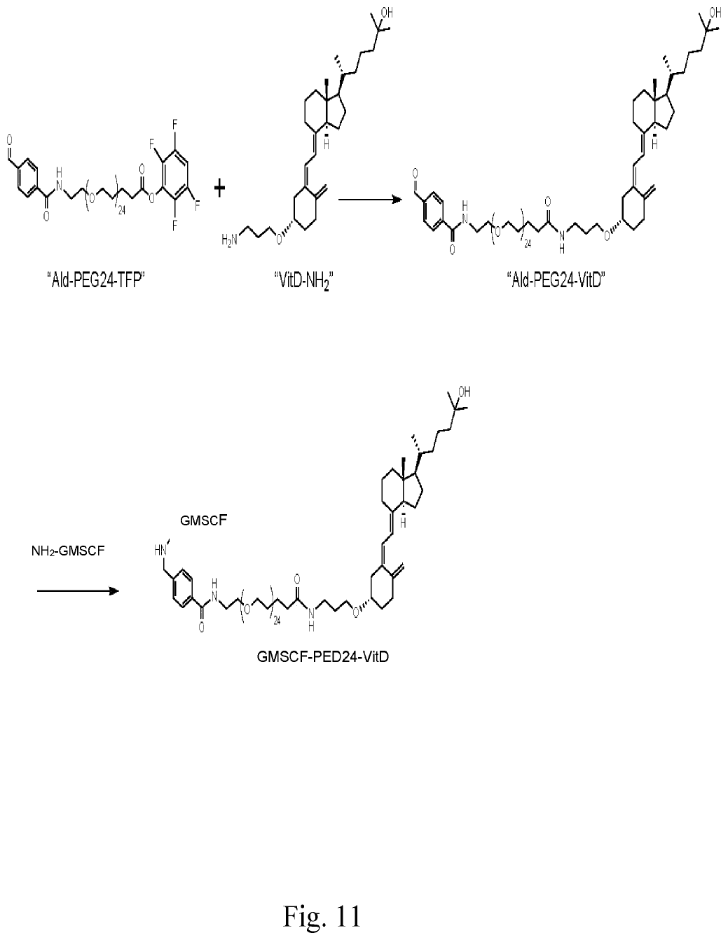

GM-CSF will be modified with compound #0-9851, which is VitD-PEG₃₆-NHS with a molecular weight of 2,356 g/mol. The NHS group (N-hydroxysuccinimide) of 0-9851 is reactive with amines on GM-CSF, of which there are seven. Test reactions will be performed with different ratios of 0-9851 to GM-CSF such that on average, just over one molecule of 0-9851 is added to each GM-CSF, as characterized by MALDI-TOF mass spectrometry and SDS-PAGE (gel electrophoresis). See FIG. 11.

In Vitro Activity:

GM-CSF and VitD-PEG-GM-CSF will be assayed for induction of receptor heterodimerization in a cell line expressing the GM-CSF receptors CSF2RB and CSF2RA. The assay will be performed by Eurofins Discovery/DiscoverX.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

The terms about, approximately, substantially, and their equivalents may be understood to include their ordinary or customary meaning. In addition, if not defined throughout the specification for the specific usage, these terms can be generally understood to represent values about but not equal to a specified value. For example, 1%, 0.9%, 0.8%, 0.7%, 0.6%, 0.5%, 0.4%, 0.3%, 0.2%, 0.1%, 0.09% of a specified value.

The terms, comprise, include, and/or plural forms of each are open ended and include the listed items and can include additional items that are not listed. The phrase “And/or” is open ended and includes one or more of the listed items and combinations of the listed items.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A carrier-drug conjugate, comprising a targeting group that is vitamin D that is not hydroxylated at the Carbon 1 position stably linked to a therapeutic compound having G-CSF activity.

2. A carrier-drug conjugate of claim 1 comprising a targeting group that is vitamin D that is not hydroxylated at the Carbon 1 position stably linked at Carbon 3 to a therapeutic compound having G-CSF activity.

3. A carrier-drug conjugate comprising a targeting group that is vitamin D that is not hydroxylated at the Carbon 1 position stably linked to a therapeutic compound having GM-CSF activity.

4. A carrier-drug conjugate of claim 3 comprising a targeting group that is vitamin D that is not hydroxylated at the Carbon 1 position stably linked at Carbon 3 to a therapeutic compound having GM-CSF activity.

5. A pharmaceutical composition comprising the carrier-drug conjugate of claim 1.

6. A pharmaceutical composition comprising the carrier-drug conjugate of claim 2.

7. A pharmaceutical composition comprising the carrier-drug conjugate of claim 3.

8. A pharmaceutical composition comprising the carrier-drug conjugate of claim 4.

9. A pharmaceutical composition comprising a mixture of a carrier-drug conjugate, comprising a targeting group that is vitamin D that is not hydroxylated at the Carbon 1 position stably linked to a therapeutic compound having G-CSF activity and a carrier-drug conjugate comprising a targeting group that is vitamin D that is not hydroxylated at the Carbon 1 position stably linked to a therapeutic compound having G-CSF activity.

10. A method of treating an animal or human patient in need of treatment with a drug having G-CSF activity, comprising administering an effective amount of the pharmaceutical composition of claim 1.

11. A method of treating an animal or human patient in need of treatment with a drug having GM-CSF activity, comprising administering an effective amount of the pharmaceutical composition of claim 2.

12. A method of treating an animal or human patient in need of treatment having G-CSF or GM-CSF activity, comprising administering effective amounts of the pharmaceutical composition having a carrier-drug conjugate, selected from the group consisting of: a carrier-drug conjugate having a targeting group that is vitamin D that is not hydroxylated at the Carbon 1 position stably linked to a therapeutic compound having G-CSF activity; a carrier-drug conjugate having a targeting group that is vitamin D that is not hydroxylated at the Carbon 1 position stably linked at Carbon 3 to a therapeutic compound having G-CSF activity; a carrier-drug conjugate having a targeting group that is vitamin D that is not hydroxylated at the Carbon 1 position stably linked to a therapeutic compound having GM-CSF activity; a carrier-drug conjugate having a targeting group that is vitamin D that is not hydroxylated at the Carbon 1 position stably linked at Carbon 3 to a therapeutic compound having GM-CSF activity; and a combination thereof.

13. The method of manufacturing the pharmaceutical composition of claim 1 comprising conjugating said targeting group and a compound having G-CSF activity.

14. The method of manufacturing the pharmaceutical composition of claim 3 comprising conjugating said targeting group and a compound having G-CSF activity.


SEQUENCE LISTING (17 sequences)

SEQ ID NO: 1 - DNA, 2374 bp, Homo sapiens
agcccggagcctgcagcccagccccacccagacccatggctggacctgccacccagagccccatgaagctgatgggtgagtgtcttggcccaggatgggagagccgcctgccctggcatgggagggaggctggtgtgacagaggggctggggatccccgttctgggaatggggattaaaggcacccagtgtccccgagagggcctcaggtggtagggaacagcatgtctcctgagcccgctctgtccccagccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgaggtgggtgagagggctgtggagggaagcccggtggggagagctaagggggatggaactgcagggccaacatcctctggaagggacgtgggagaatattaggagcagtggagctggggaaggctgggaagggacttggggaggaggaccttggtggggacagtgctcgggagggctggctgggatgggagtggaggcattacattcaggagaaagggcaagggcccctgtgagatcagagagtgggggtgcagggcagagaggaactgaacagcctggcaggacatggagggaggggaaagaccagagagtcggggaggacccgggaaggagcggcgacccggccatggcgagtctcactcagcatccttccatccccagtgtgccacctacaagctgtgccaccccgaggagctggtgctgctcggacactctctgggcatcccctgggctcccctgagcagctgccccagccaggccctgcagctggtgagtgtcaggaaaggataaggctaatgaggagggggaaggagaggaggaacacccatgggctcccccatgtctccaggttccaagctgggggcctgacgtatctcaggcagcaccccctaactcttccgctctgtctcacaggcaggctgcttgagccaactccatagcggccttttcctctaccaggggctcctgcaggccctggaagggatctcccccgagttgggtcccaccttggacacactgcagctggacgtcgccgactttgccaccaccatctggcagcaggtgagccttgttgggcagggtggccaaggtcgtgctggcattctgggcaccacagccaggcctgtgtatgggccctgtccatgctgtcacccccagcatttcctcatttgtaataacgcccactcagaagggcccaaccactgatcacagctttcccccacagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctcca

SEQ ID NO: 2 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - DNA, 3008 bp, Homo sapiens
agagctcggggagagaagctggactgcagctggtttcaggaacttctcttgacgagaagagagaccaaggaggccaagcaggggctgggccagaggtgccaacatggggaaactgaggctcggctcggaaaggtgaagtaacttgtccaagatcacaaagctggtgaacatcaagttggtgctatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccatccagccccacccaatggccttttgtgcttgtttcctataacttcagtattgtaaactagtttttggtttgcagtttttgttgttgtttatagacactcttgggtgta

SEQ ID NO: 4 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 5 - DNA, 660 bp, Homo sapiens
aaaaaacagcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgagcc

SEQ ID NO: 6 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 624 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctga

SEQ ID NO: 8 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 9 - DNA, 3194 bp, Homo sapiens
aagcttgctgagagtggctgcagtctcgctgctggatgtgcacatggtggtcattccctctgctcacaggggcaggggtccccccttactggactcaggttgccccctgctccaggtcctgggtgggagcccatgtgaactgtcagtggggcaggtctgtgagagctcccctcacactcaagtctctcacagtggccagagaagaggaaggctggagtcagaatgaggcaccagggcgggcatagcctgcccaaaggcccctgggattacaggcaggatggggagccctatctaagtgtctcccacgccccaccccagccattccaggccaggaagtccaaactgtgcccctcagagggagggggcagcctcaggcccattcagactgcccagggagggctggagagccctcaggaaggcgggtgggtgggctgtcggttcttggaaaggttcattaatgaaaacccccaagcctgaccacctagggaaaaggctcaccgttcccatgtgtggctgataagggccaggagattccacagttcaggtagttcccccgcctccctggcattttgtggtcaccattaatcatttcctctgtgtatttaagagctcttttgccagtgagcccagtacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatggtaagtgagagaatgtgggcctgtgcctaggccacccagctggcccctgactggccacgcctgtcagcttgataacatgacattttccttttctacagaatgaaacagtagaagtcatctcagaaatgtttgacctccaggtaagatgcttctctctgacatagctttccagaagcccctgccctggggtggaggtggggactccattttagatggcaccacacagggttgtccactttctctccagtcagctggctgcaggaggagggggtagcaactgggtgctcaagaggctgctggccgtgcccctatggcagtcacatgagctcctttatcagctgagcggccatgggcagacctagcattcaatggccaggagtcaccaggggacaggtggtaaagtgggggtcacttcatgagacaggagctgtgggtttggggcgctcactgtgccccgagaccaagtcctgttgagacagtgctgactacagagaggcacagaggggtttcaggaacaacccttgcccacccagcaggtccaggtgaggccccacccccctctccctgaatgatggggtgagagtcacctccttccctaaggctgggctcctctccaggtgccgctgagggtggcctgggcggggcagtgagaagggcaggttcgtgcctgccatggacagggcagggtctatgactggacccacgctgtgcccctcccaagccctactcctgggggctgggggcagcagcaaaaaggagtggtggagagttcttgtaccactgtgggcacttggccactgctcaccgacgaacgacattttccacaggagccgacctgcctacagacccgcctggagctgtacaagcagggcctgcggggcagcctcaccaagctcaagggccccttgaccatgatggccagccactacaagcagcactgccctccaaccccggtgagtgcctacggcagggcctccagcaggaatgtcttaatctagggggtggggtcgacatggggagagatctatggctgtggctgttcaggaccccagggggtttctgtgccaacagttatgtaatgattagccctccagagaggaggcagacagcccatttcatcccaaggagtcagagccacagagcgctgaagcccacagtgctccccagcaggagctgctcctatcctggtcattattgtcattacggttaatgaggtcagaggtgagggcaaacccaaggaaacttggggcctgcccaaggcccagaggaagtgcccaggcccaagtgccaccttctggcaggactttcctctggccccacatggggtgcttgaattgcagaggatcaaggaagggaggctacttggaatggacaaggacctcaggcactccttctgcgggaagggagcaaagtttgtggccttgactccactccttctgggtgcccagagacgacctcagcccagctgccctgctctgccctgggaccaaaaaggcaggcgtttgactgcccagaaggccaacctcaggctggcacttaagtcaggcccttgactctggctgccactggcagagctatgcactccttggggaacacgtgggtggcagcagcgtcacctgacccaggtcagtgggtgtgtcctggagtgggcctcctggcctctgagttctaagaggcagtagagaaacatgctggtgcttccttcccccacgttacccacttgcctggactcaagtgttttttatttttctttttttaaaggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttgtccagtgttctagtttgtttttaaccatgagcaaatgccagtgggtgcctgccttcccatgaggcaggggagggaggaaacggggaggtggagagggggcgggggcctcccaggcgttgggcactatccaagggccaacactgtcagagcagaggggaggtgagagccgggcatagtcggaattc

SEQ ID NO: 10 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 11 - DNA, 788 bp, Homo sapiens
agtacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttg

SEQ ID NO: 12 - protein, 144 aa, Homo sapiens; identical to SEQ ID NO: 10

SEQ ID NO: 13 - protein, 128 aa, Homo sapiens
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 14 - DNA, 1801 bp, Homo sapiens
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 15 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 16 - protein, 35 aa, Homo sapiens
SVSEIQLMHNLGKHLNSMERVEWLRKKLQDVHNFC

SEQ ID NO: 17 - protein, 35 aa, Homo sapiens
CSVSEIQLMHNLGKHLNSMERVEWLRKKLQDVHNF